SEQUENCE LISTING (6 sequences)

SEQ ID NO: 1 - DNA, 116 bp, Artificial
  Synthetic
  misc_feature (48)..(53): n is a, c, g, or t
  misc_feature (72)..(86): n is a, c, g, or t
gccggtaatacgactcactatagggctacacgacgctcttccgatctnnnnnntcttcagcgttcccgagannnnnnnnnnnnnnntttttttttttttttttttttttttttttt

SEQ ID NO: 2 - DNA, 108 bp, Artificial
  Synthetic
  misc_feature (38)..(45): n is a, c, g, or t
  misc_feature (64)..(78): n is a, c, g, or t
ttttttttgccggggctacacgacgctcttccgatctnnnnnnnntcttcagcgttcccgagannnnnnnnnnnnnnntttttttttttttttttttttttttttttt

SEQ ID NO: 3 - DNA, 58 bp, Artificial
  Synthetic
aatgatacggcgaccaccgagatctacactctttccctacacgacgctcttccgatct

SEQ ID NO: 4 - DNA, 23 bp, Artificial
  Synthetic
aagcagtggtatcaacgcagagt

SEQ ID NO: 5 - DNA, 22 bp, Artificial
  Synthetic
ctacacgacgctcttccgatct

SEQ ID NO: 6 - DNA, 28 bp, Artificial
  Synthetic
aagctggtatcaacgcagagtgaatggg

(19) United States
(12) Patent Application Publication
(10) Pub. No.: US 2022/0177963 A1
(43) Pub. Date: Jun. 9, 2022

(54) PAIRED MACROMOLECULE ABUNDANCE AND T-CELL RECEPTOR SEQUENCING WITH HIGH SPATIAL RESOLUTION

(71) Applicants: THE BROAD INSTITUTE, INC., Cambridge, MA (US); PRESIDENT AND FELLOWS OF HARVARD COLLEGE, Cambridge, MA (US)

(72) Inventors: Fei Chen, Cambridge, MA (US); Sophia Liu, Cambridge, MA (US)

(73) Assignees: THE BROAD INSTITUTE, INC., Cambridge, MA (US); PRESIDENT AND FELLOWS OF HARVARD COLLEGE, Cambridge, MA (US)

(21) Appl. No.: 17/542,650

(22) Filed: Dec. 6, 2021

Related U.S. Application Data
(60) Provisional application No. 63/122,357, filed on Dec. 7, 2020.

(51) Int. Cl. C12Q 1/6874; G01N 1/30; C12Q 1/6837; C12Q 1/686; C12Q 1/6804; G01N 33/543; C12Q 1/6876

(52) U.S. Cl. CPC C12Q 1/6874; G01N 1/30; C12Q 1/6837; C12Q 2600/158; C12Q 1/6804; G01N 33/54353; C12Q 1/6876; C12Q 1/686

(57) ABSTRACT

The present disclosure relates to compositions and methods for assessing extended length T-cell receptor (TCR) transcript sequences (i.e., TCR transcript sequences that span TCR transcript variable regions) in a spatially-defined manner across a tissue sample, specifically providing for obtaining useful TCR sequences at high spatial resolution while also assessing relative macromolecule abundance (e.g., RNA expression levels) with deep transcriptomic coverage at similarly high-resolution across the tissue sample.

### CROSS-REFERENCE TO RELATED APPLICATION

The present application is related to and claims priority under 35 U.S.C. § 119(e) to U.S. provisional patent application No. 63/122,357, entitled “Paired Macromolecule Abundance and T-Cell Receptor Sequencing with High Spatial Resolution,” filed Dec. 7, 2020. The entire content of the aforementioned patent application is incorporated herein by this reference.

### STATEMENT REGARDING FEDERALLY SPONSORED RESEARCH

This invention was made with government support under Grant No. AI142737 awarded by the National Institutes of Health. The government has certain rights in the invention.

### FIELD OF THE INVENTION

The invention relates generally to methods and compositions for coordinated spatial assessment of both T-cell receptor (TCR) sequence and macromolecule abundance (e.g., RNA expression, DNA abundance, protein abundance) in a tissue sample.

### SEQUENCE LISTING

The instant application contains a Sequence Listing which has been filed electronically in ASCII format and is hereby incorporated by reference in its entirety. Said ASCII copy, created on Dec. 3, 2021, is named BN00007_1301_BI_10782_SL.txt and is 2 KB in size.

### BACKGROUND OF THE INVENTION

An improved approach for obtaining spatial macromolecule abundance data (e.g., RNA expression, DNA and/or protein abundance) at resolutions approaching single cell resolution was previously described, in international application no. PCT/US19/30194. Extension of the approach described therein to allow for enhanced obtainment of spatially refined T-cell receptor transcript sequences of sufficient length to resolve the TCR variable region, together with a spatially refined view of associated macromolecule abundance, is desirable.

### BRIEF SUMMARY OF THE INVENTION

The instant disclosure is based, at least in part, upon discovery of a method for obtaining robust T-cell receptor (TCR) transcript sequence data, at read lengths sufficient to resolve the TCR transcript variable region, in a manner that is spatially localized and at near single-cell resolution, while also collecting associated, spatially resolved macromolecule abundance information. Accordingly, certain aspects of the instant disclosure address how to sequence T-cell receptors (TCRs) while retaining their spatial origins in tissue at high resolution.

In one aspect, the instant disclosure provides a method for obtaining from a tissue sample spatially-resolvable T cell receptor (TCR) sequence that spans TCR variable regions, the method involving: (i) obtaining a tissue sample from a subject; (ii) preparing a section of the tissue sample; (iii) providing a solid support; (iv) contacting the solid support with a capture material, thereby forming a capture material-coated solid support; (v) contacting the capture material-coated solid support with a population of 1-100 μm diameter beads, where each bead has at least 1000 attached oligonucleotides and where at least one attached oligonucleotide of each bead each includes: (a) a bead identification sequence that is common to all at least 1000 oligonucleotides on each bead and (b) a poly-dT tail of sufficient length to allow for capture of poly-A-tailed RNAs via hybridization, where the bead identification sequence that is common to all at least 1000 oligonucleotides on each bead is either a bead identification sequence that is unique to each bead within the population of 1-100 μm diameter beads or is a bead identification sequence that is a member of a population of bead identification sequences that is sufficiently degenerate to the population of 1-100 μm diameter beads that a majority of beads within the population of 1-100 μm diameter beads each possesses a unique bead identification sequence, thereby capturing a subpopulation of the population of 1-100 μm diameter beads upon the solid support; (vi) identifying the bead identification sequence and associated two-dimensional position on the solid support of individual beads of the subpopulation of beads attached to the solid support; (vii) contacting the subpopulation of 1-100 μm diameter beads captured upon the solid support with the section of the tissue sample; (viii) performing a reverse transcription reaction upon poly-A-tailed RNAs captured by the bead subpopulation, thereby generating a cDNA population; (ix) contacting a selection of or all of the cDNA population with (a) RNase H-dependent PCR primers designed for specific amplification of TCR-alpha and TCR-beta cDNAs and (b) RNase H, and performing PCR amplification upon the cDNA population, thereby generating a PCR-amplified nucleic acid population enriched for TCR-alpha and TCR-beta sequences; and (x) obtaining sequence from the PCR-amplified nucleic acid population enriched for TCR-alpha and TCR-beta sequences using a sequencing process upon TCR-containing sequences having an average read length in excess of 200 nucleotides on at least one end of such sequences (e.g., in some embodiments where paired end sequencing is used, the TCR sequence-containing end of a cDNA or amplicon is sequenced using a process that obtains an average read length of 200 nucleotides or more (a length sufficient to resolve TCR clonotypes), while the spatial identifier end of a sequenced fragment is sequenced using a process that provides an average read length of 20 or more nucleotides, 30 or more nucleotides, 40 or more nucleotides, at least 50 nucleotides, or more), thereby obtaining TCR sequences that span TCR variable regions for substantially all TCR sequences obtained and obtaining sequence from the PCR-amplified nucleic acid population enriched for TCR-alpha and TCR-beta sequences of bead identification sequences associated with TCR sequences, thereby obtaining spatially-resolvable T cell receptor (TCR) sequence that spans TCR variable regions from the tissue sample.

In certain embodiments, each bead has at least 1000 attached oligonucleotides, where at least 100, and optionally at least 1000, attached oligonucleotides of each bead each includes: (a) a bead identification sequence that is common to all at least 1000 oligonucleotides on each bead and (b) a poly-dT tail of sufficient length to allow for capture of poly-A-tailed RNAs via hybridization.

In embodiments, PCR amplification is performed upon the cDNA population of step (viii) in a manner that does not specifically enrich for TCR-alpha and TCR-beta sequences, thereby generating a PCR-amplified cDNA population that is not specifically enriched for TCR-alpha and TCR-beta sequences, where the PCR-amplified cDNA population that is not specifically enriched for TCR-alpha and TCR-beta sequences, or a subpopulation thereof, is the cDNA population contacted in step (ix) with (a) RNase H-dependent PCR primers designed for specific amplification of TCR-alpha and TCR-beta cDNAs and (b) RNase H, thereby generating a PCR-amplified nucleic acid population enriched for TCR-alpha and TCR-beta sequences.

In one embodiment, the cDNA population of step (viii) is partitioned into a first selection of the cDNA population that is contacted in step (ix) with RNase H-dependent PCR primers designed for specific amplification of TCR-alpha and TCR-beta cDNAs and RNase H, thereby generating a first PCR-amplified nucleic acid population that is enriched for TCR-alpha and TCR-beta sequences, and a second selection of the cDNA population. Optionally, the second selection of the cDNA population is amplified with primers that are not selective for TCR sequence, thereby generating a second PCR-amplified nucleic acid population that is not enriched for TCR sequence relative to the cDNA population of step (viii).

In a related embodiment, the PCR-amplified nucleic acid population enriched for TCR-alpha and TCR-beta sequences and the PCR-amplified nucleic acid population that is not specifically enriched for TCR-alpha and TCR-beta sequences are combined prior to obtaining sequence from the PCR-amplified nucleic acid population using a sequencing process having, at least for one end of TCR sequence-containing nucleic acids, an average read length in excess of 200 nucleotides in step (x), where sequences of non-TCR transcripts and associated bead identification sequences are thereby also obtained in step (x), where the method thereby obtains both spatially-resolvable T cell receptor (TCR) sequence that spans TCR variable regions and spatially-resolvable transcript abundance information from the tissue sample.

In embodiments, the PCR-amplified nucleic acid population enriched for TCR-alpha and TCR-beta sequences, and optionally the PCR-amplified nucleic acid population that is not specifically enriched for TCR-alpha and TCR-beta sequences, is cleaved and tagged prior to obtaining sequence from the PCR-amplified nucleic acid population in step (x).

In some embodiments, bead identification sequences associated with transcripts are obtained using paired-end sequencing. Optionally, sequences of bead identification sequences associated with TCR sequences are obtained using paired-end sequencing.

In certain embodiments, a subpopulation of the at least 1000 attached oligonucleotides of each bead includes (a) a bead identification sequence that is common to all at least 1000 oligonucleotides on each bead and (b) a macromolecule-specific capture sequence that does not include a poly-dT tail.

In a related embodiment, the macromolecule is RNA, DNA or protein.

In embodiments, the macromolecule-specific capture sequence includes a gene-specific or transcript-specific sequence.

In one embodiment, the DNA is a genomic DNA, a barcode DNA, or both.

In some embodiments, the macromolecule-specific capture sequence is a component of a loaded transposase.

In one embodiment, a DNA barcode is used to capture an attached protein. Optionally, the barcode-attached protein is an antibody. Optionally, the antibody is specifically bound to a target protein. Optionally, the antibody-bound target protein includes a label.

In embodiments, the method further involves PCR amplifying a nucleotide sequence of the captured macromolecule, thereby generating a PCR-amplified macromolecule nucleotide sequence population, and obtaining sequence from the PCR-amplified macromolecule nucleotide sequence population, thereby also obtaining spatially-resolvable macromolecule abundance data from the tissue sample.

In some embodiments, the PCR-amplified nucleic acid population including TCR-alpha and TCR-beta sequences is cleaved and tagged before obtaining sequence from the PCR-amplified nucleic acid population in step (x). Optionally, a second PCR-amplified nucleic acid population is also cleaved and tagged before also obtaining sequence from the second PCR-amplified nucleic acid population.

In certain embodiments, obtaining sequence from the PCR-amplified nucleic acid population in step (x) is performed using a next-generation sequencing (NGS) method. Optionally, the NGS sequencing method is solid-phase, reversible dye-terminator sequencing; massively parallel signature sequencing; pyro-sequencing; sequencing-by-ligation; ion semiconductor sequencing; Nanopore sequencing or DNA nanoball sequencing. Optionally, the next-generation sequencing approach is solid-phase, reversible dye-terminator sequencing.

In some embodiments, obtaining sequence from the PCR-amplified nucleic acid population in step (x) is performed using a long read sequencing (LRS) method. Optionally, the LRS method is single molecule real time sequencing (SMRT) or nanopore sequencing.

In embodiments, the average read length of the sequencing process employed (e.g., a standard NGS approach adapted to obtain extended read lengths, or a true LRS approach) exceeds about 850 nucleotides. Optionally, the average read length of the sequencing process exceeds about 900 nucleotides. Optionally, the average read length of the sequencing process exceeds about 950 nucleotides. Optionally, the average read length of the sequencing process exceeds about 1000 nucleotides. Optionally, the average read length of the sequencing process exceeds about 1050 nucleotides. Optionally, the average read length of the sequencing process exceeds about 1100 nucleotides. Optionally, the average read length of the sequencing process exceeds about 1150 nucleotides. Optionally, the average read length of the sequencing process exceeds about 1200 nucleotides. Optionally, the average read length of the sequencing process exceeds about 1250 nucleotides. Optionally, the average read length of the sequencing process exceeds about 1300 nucleotides.

In certain embodiments, the tissue sample is obtained from brain, lung, liver, kidney, pancreas, heart, spleen, lymph node, thymus, or tumor.

In embodiments, the subject is a mammal. Optionally, the subject is a human.

In some embodiments, the tissue sample is fixed. Optionally, the tissue sample is fixed with formalin, methanol, ethanol, and/or acetone. Optionally, the tissue sample is a formalin-fixated and paraffin-embedded (FFPE) pathology specimen.

In embodiments, the solid support is a slide. Optionally, the solid support is a glass slide.

In certain embodiments, the capture material is applied as a liquid. Optionally, the capture material is applied using a brush or aerosol spray. Optionally, the capture material is a liquid electrical tape. Optionally, the capture material dries to form a vinyl polymer. Optionally, the vinyl polymer is polyvinyl hexane.

In some embodiments, the 1-100 μm diameter beads include porous polystyrene, porous polymethacrylate and/or polyacrylamide.

In embodiments, the beads are 1-40 μm diameter beads. Optionally, the beads are 10 μm beads.

In one embodiment, the step of (vi) identifying the bead identification sequence and associated two-dimensional position on the solid support of individual beads of the subpopulation of beads attached to the solid support includes performance of a sequencing-by-ligation technique.

In some embodiments, the subpopulation of 1-100 μm diameter beads captured upon the solid support in step (vii) is maintained at a temperature between 4° C. and 30° C. Optionally, at about 25° C.

In embodiments, step (vii) further includes contacting the subpopulation of 1-100 μm diameter beads captured upon the solid support with a wash solution. Optionally, with a saline solution. Optionally, with a solution including between about 1M and about 3M NaCl. Optionally, with a saline-sodium citrate buffer including between about 1M and about 3M NaCl.

In certain embodiments, the bead identification sequence and associated two-dimensional position on the solid support of individual beads of the subpopulation of beads attached to the solid support is registered in a computer.

In some embodiments, the method further involves step (xi) generating an image of the tissue sample that depicts the location(s) and relative abundance of one or more captured TCRs and/or other captured macromolecules within the sample. Optionally, the image is a two-dimensional image.

In embodiments, the hybridization is performed in 6×SSC buffer. Optionally, the 6×SSC buffer is supplemented with detergent.

In another embodiment, a selection of the beads possess primers against specific transcripts.

In certain embodiments, the barcoded array is reusable. Optionally, cDNA is generated and then the second strand (carrying the barcode location) is synthesized. Optionally, the second strand is capable of release from the array. Optionally, the cDNA can be cleaved using a restriction enzyme to reveal a poly(A) tail on the array, thereby allowing for the array to be reused.

In one embodiment, transcript-specific amplification of one or more transcripts other than TCR transcripts is also performed.

Another aspect of the instant disclosure provides a method for obtaining from a tissue sample spatially-resolvable TCR sequence that spans TCR variable regions and spatially-resolvable bulk poly-A-tailed RNA expression data, the method involving: (i) obtaining a tissue sample from a subject; (ii) preparing a section of the tissue sample; (iii) obtaining a solid support; (iv) contacting the solid support with a capture material, thereby forming a capture material-coated solid support; (v) contacting the capture material-coated solid support with a population of 1-100 μm diameter beads, where each bead has at least 1000 attached oligonucleotides and where at least 1000 attached oligonucleotides of each bead each includes: (a) a bead identification sequence that is common to all at least 1000 oligonucleotides on each bead and (b) a poly-dT tail of sufficient length to allow for capture of poly-A-tailed RNAs via hybridization, where the bead identification sequence that is common to all at least 1000 oligonucleotides on each bead is either a bead identification sequence that is unique to each bead within the population of 1-100 μm diameter beads or is a bead identification sequence that is a member of a population of bead identification sequences that is sufficiently degenerate to the population of 1-100 μm diameter beads that a majority of beads within the population of 1-100 μm diameter beads each possesses a unique bead identification sequence, thereby capturing a subpopulation of the population of 1-100 μm diameter beads upon the solid support; (vi) identifying the bead identification sequence and associated two-dimensional position on the solid support of individual beads of the subpopulation of beads attached to the solid support; (vii) contacting the subpopulation of 1-100 μm diameter beads captured upon the solid support with the section of the tissue sample; (viii) performing a reverse transcription reaction upon poly-A-tailed RNAs captured by the bead subpopulation, thereby generating a cDNA population; (ix) performing PCR amplification upon the cDNA subpopulation in a manner that does not specifically enrich for TCR-alpha and TCR-beta sequences, thereby generating a PCR-amplified nucleic acid population not specifically enriched for TCR-alpha and TCR-beta sequences; (x) contacting the PCR-amplified nucleic acid population not specifically enriched for TCR-alpha and TCR-beta sequences, or a subpopulation thereof, with (a) RNase H-dependent PCR primers designed for specific amplification of TCR-alpha and TCR-beta cDNAs and (b) RNase H, and performing PCR amplification, thereby generating a PCR-amplified nucleic acid population enriched for TCR-alpha and TCR-beta sequences; (xi) combining the PCR-amplified nucleic acid population enriched for TCR-alpha and TCR-beta sequences and the PCR-amplified nucleic acid population not specifically enriched for TCR-alpha and TCR-beta sequences into a single PCR-amplified nucleic acid population; and (xii) obtaining sequence from the PCR-amplified nucleic acid population using a sequencing process having an average read length for at least one end of TCR-containing sequences in excess of 200 nucleotides, thereby obtaining (a) TCR sequences that span TCR variable regions for substantially all TCR sequences obtained; (b) sequences of bead identification sequences associated with TCR sequences; and (c) sequences of a population of poly-A-tailed RNAs bound to the bead oligonucleotides and associated bead identification sequences for sequenced poly-A-tailed RNAs, thereby obtaining spatially-resolvable T cell receptor (TCR) sequence that spans TCR variable regions and spatially-resolvable bulk poly-A-tailed RNA expression data from the tissue sample.

An additional aspect of the instant disclosure provides a method for obtaining from a tissue sample spatially-resolvable TCR sequence that spans TCR variable regions, the method involving: (i) generating a well array, where each well of the array can hold exactly one bead; (ii) depositing beads into the wells of the well array, optionally by evaporation in a centrifuge; (iii) brushing the well array to remove all of the beads not present in wells; (iv) obtaining a tissue sample from a subject; (v) preparing a section of the tissue sample; (vi) depositing the section onto the well array and centrifuging, thereby forcing the section into the wells of the well array; (vii) adding digestion buffer, thereby lysing the section and causing the RNA of cells of the section to transfer onto the beads in the wells; (viii) performing a reverse transcription reaction upon the beads in the wells, thereby generating a cDNA population; (ix) contacting a selection of or all of the cDNA population with (a) RNase H-dependent PCR primers designed for specific amplification of TCR-alpha and TCR-beta cDNAs and (b) RNase H, and performing PCR amplification upon the cDNA population, thereby generating a PCR-amplified nucleic acid population including TCR-alpha and TCR-beta sequences; and (x) obtaining sequence from the PCR-amplified nucleic acid population using a sequencing process having an average read length for at least one end of TCR-containing sequences in excess of 200 nucleotides, thereby obtaining TCR sequences that span TCR variable regions (at least to an extent sufficient for such TCR variable regions) for substantially all TCR sequences obtained and obtaining sequence from the PCR-amplified nucleic acid population of bead identification sequences associated with TCR sequences, thereby obtaining spatially-resolvable T cell receptor (TCR) sequence that spans TCR variable regions from the tissue sample.

In certain embodiments, the method further involves removing beads from the wells by sonication or by photocleavage after step (vii). Optionally, removing beads from the wells by sonication or by photocleavage after step (vii) occurs before performing step (viii).

Another aspect of the instant disclosure provides a method for obtaining from a tissue sample spatially-resolvable TCR sequence that spans TCR variable regions, the method involving: (i) obtaining a tissue sample from a subject; (ii) preparing a section of the tissue sample; (iii) obtaining a solid support; (iv) adhering clusters of oligonucleotides in an array attached to the solid support; (v) identifying oligonucleotide cluster identification sequences and associated two-dimensional positions on the solid support of individual oligonucleotide clusters attached to the solid support, where the individual oligonucleotides are designed to capture RNA or DNA from the section of the tissue sample, optionally where at least one of the individual oligonucleotides of each cluster is designed for specific capture of TCR mRNA from the section of the tissue sample; (vii) contacting the array with the section of the tissue sample; (viii) performing RNase H-dependent PCR upon captured mRNAs of the section of the tissue sample, thereby generating a PCR-amplified DNA population including TCR-alpha and TCR-beta sequences; and (ix) obtaining sequence from the PCR-amplified DNA population and an associated oligonucleotide cluster identification sequence for each DNA sequenced using a sequencing process having an average read length at least for one end of TCR-containing sequences that is in excess of 200 nucleotides, thereby obtaining TCR sequences that span TCR variable regions (to an extent sufficient for resolution of such TCR variable regions) for substantially all TCR sequences obtained and obtaining sequence from the PCR-amplified nucleic acid population of bead identification sequences associated with TCR sequences, thereby obtaining spatially-resolvable TCR sequence that spans TCR variable regions from the tissue sample.

In embodiments, the array includes barcoded clusters of oligonucleotides on a surface.

Another aspect of the instant disclosure provides a method for obtaining from a tissue sample spatially-resolvable TCR sequence that spans TCR variable regions and macromolecule abundance data, the method involving: (i) obtaining a tissue sample from a subject; (ii) preparing a section of the tissue sample and adhering the section to a solid support; (iii) forming an array of barcoded oligonucleotide clusters and/or an array of beads attached to barcoded oligonucleotides and contacting the section adhered to the solid support with the array; (iv) identifying oligonucleotide cluster and/or bead array identification sequences and associated two-dimensional positions on the array of the barcoded oligonucleotide clusters and/or the array of beads attached to barcoded oligonucleotides; and (v) obtaining the sequences of a population of macromolecules bound to the array(s) for each macromolecule sequenced, where the population of macromolecules includes TCR RNA sequences, where TCR sequences are obtained by a process involving RNase H-dependent PCR amplification of captured TCR RNA, thereby generating a PCR-amplified cDNA population including TCR-alpha and TCR-beta sequences, and obtaining sequence of the PCR-amplified cDNA population and an associated oligonucleotide cluster identification sequence for each cDNA sequenced using a sequencing process having an average read length for at least one end of TCR sequence cDNAs in excess of 200 nucleotides, thereby obtaining TCR sequences that span TCR variable regions for substantially all TCR sequences obtained and obtaining sequence from the PCR-amplified cDNA population of oligonucleotide cluster and/or bead array identification sequences associated with TCR sequences, thereby obtaining spatially-resolvable TCR sequence that spans TCR variable regions and macromolecule abundance data from the tissue sample.

In embodiments, an array (puck) is physically transferred from one surface to another. Optionally, a gel encasement is formed on top of the array (puck), thereby allowing beads to be picked up off the surface of the array (puck) without altering bead positions relative to each other.

In some embodiments, the beads or array include or bind oligonucleotide-conjugated antibodies.

In certain embodiments, the oligonucleotides having a poly-dT tail of sufficient length to allow for capture of poly-A-tailed RNAs via hybridization include unique molecular identifiers (UMIIs). Optionally, the UMIIs of the oligonucleotides having a poly-dT tail of sufficient length to allow for capture of poly-A-tailed RNAs via hybridization are counted via sequencing to assess the levels of hybridization probe-bound macromolecules. Optionally, the hybridization probe-bound macromolecules are selected from the group consisting of proteins, exons, transcripts, nucleic acid sequences including single nucleotide polymorphisms (SNPs) and/or genomic regions.

While RNase H-dependent PCR amplification is exemplified herein for enriching for TCR sequences, it is further contemplated that probe sequences specific for TCR sequences can also be employed for such TCR sequence enrichment. Thus, in an alternative aspect of the instant disclosure, biotin-tagged probes specific for TCR-alpha or TCR-beta sequences can be used for enrichment of TCR-containing sequences, via streptavidin-biotin-mediated binding and (optionally) pulldown of TCR-containing sequences. Accordingly, a further aspect of the instant disclosure provides a method for obtaining from a tissue sample spatially-resolvable T cell receptor (TCR) sequence that spans TCR variable regions, the method involving: (i) obtaining a tissue sample from a subject; (ii) preparing a section of the tissue sample; (iii) providing a solid support; (iv) contacting the solid support with a capture material, thereby forming a capture material-coated solid support; (v) contacting the capture material-coated solid support with a population of 1-100 μm diameter beads, where each bead has at least 1000 attached oligonucleotides and where at least one attached oligonucleotide of each bead each includes: (a) a bead identification sequence that is common to all at least 1000 oligonucleotides on each bead and (b) a poly-dT tail of sufficient length to allow for capture of poly-A-tailed RNAs via hybridization, where the bead identification sequence that is common to all at least 1000 oligonucleotides on each bead is either a bead identification sequence that is unique to each bead within the population of 1-100 μm diameter beads or is a bead identification sequence that is a member of a population of bead identification sequences that is sufficiently degenerate to the population of 1-100 μm diameter beads that a majority of beads within the population of 1-100 μm diameter beads each possesses a unique bead identification sequence, thereby capturing a subpopulation of the population of 1-100 μm diameter beads upon the solid support; (vi) identifying the bead identification sequence and associated two-dimensional position on the solid support of individual beads of the subpopulation of beads attached to the solid support; (vii) contacting the subpopulation of 1-100 μm diameter beads captured upon the solid support with the section of the tissue sample; (viii) performing a reverse transcription reaction upon poly-A-tailed RNAs captured by the bead subpopulation, thereby generating a cDNA population; (ix) contacting a selection of or all of the cDNA population with biotinylated probes capable of specifically annealing to TCR-alpha or TCR-beta sequences, and enriching for biotinylated probe-TCR complexes, thereby generating a nucleic acid population enriched for TCR-alpha and TCR-beta sequences; and (x) obtaining sequence from the nucleic acid population enriched for TCR-alpha and TCR-beta sequences using a sequencing process upon TCR-containing sequences having an average read length in excess of 200 nucleotides on at least one end of such sequences (e.g., in embodiments, where paired end sequencing is used, the TCR sequence-containing end is sequenced with a process that obtains an average read length of 200 nucleotides or more, while the spatial identifier sequence end is sequenced using a process that provides an average read length of 20 or more nucleotides, 30 or more nucleotides, 40 or more nucleotides, at least 50 nucleotides, or more), thereby obtaining TCR sequences that span TCR variable regions for substantially all TCR sequences obtained and obtaining sequence from the nucleic acid population enriched for TCR-alpha and TCR-beta sequences of bead identification sequences associated with TCR sequences, thereby obtaining spatially-resolvable T cell receptor (TCR) sequence that spans TCR variable regions from the tissue sample.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value.

In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Unless otherwise clear from context, all numerical values provided herein are modified by the term “about.”

As used herein, the term “amplicon,” when used in reference to a nucleic acid, means the product of copying the nucleic acid, wherein the product has a nucleotide sequence that is the same as or complementary to at least a portion of the nucleotide sequence of the nucleic acid. An amplicon can be produced by any of a variety of amplification methods that use the nucleic acid, or an amplicon thereof, as a template including, for example, polymerase extension, polymerase chain reaction (PCR), rolling circle amplification (RCA), multiple displacement amplification (MDA), ligation extension, or ligation chain reaction. An amplicon can be a nucleic acid molecule having a single copy of a particular nucleotide sequence (e.g. a PCR product) or multiple copies of the nucleotide sequence (e.g. a concatameric product of RCA). A first amplicon of a target nucleic acid is typically a complementary copy. Subsequent amplicons are copies that are created, after generation of the first amplicon, from the target nucleic acid or from the first amplicon. A subsequent amplicon can have a sequence that is substantially complementary to the target nucleic acid or substantially identical to the target nucleic acid.

As used herein, the term “array” refers to a population of features or sites that can be differentiated from each other according to relative location. Different molecules that are at different sites of an array can be differentiated from each other according to the locations of the sites in the array. An individual site of an array can include one or more molecules of a particular type. For example, a site can include a single target nucleic acid molecule having a particular sequence or a site can include several nucleic acid molecules having the same sequence (and/or complementary sequence, thereof). The sites of an array can be different features located on the same substrate.

Exemplary features include without limitation, wells in a substrate, beads (or other particles) in or on a substrate, projections from a substrate, ridges on a substrate or channels in a substrate. The sites of an array can be separate substrates each bearing a different molecule. Different molecules attached to separate substrates can be identified according to the locations of the substrates on a surface to which the substrates are associated or according to the locations of the substrates in a liquid or gel. Exemplary arrays in which separate substrates are located on a surface include, without limitation, those having beads in wells, beads arranged upon a flat surface (e.g., a slide), optionally beads captured upon a flat surface (e.g., a layer of beads adhered to or otherwise stably associated with a slide (e.g., a layer of beads adsorbed to a slide-attached elastomeric surface)), etc.

As used herein, the term “attached” refers to the state of two things being joined, fastened, adhered, connected or bound to each other. For example, an analyte, such as a nucleic acid, can be attached to a material, such as a gel or solid support, by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions and hydrophobic interactions.

As used herein, the term “barcode sequence” is intended to mean a series of nucleotides in a nucleic acid that can be used to identify the nucleic acid, a characteristic of the nucleic acid (e.g., the identity and optionally the location of a bead to which the nucleic acid is attached), or a manipulation that has been carried out on the nucleic acid. The barcode sequence can be a naturally occurring sequence or a sequence that does not occur naturally in the organism from which the barcoded nucleic acid was obtained. A barcode sequence can be unique to a single nucleic acid species in a population or a barcode sequence can be shared by several different nucleic acid species in a population (e.g., all nucleic acid species attached to a single bead might possess the same barcode sequence, while different beads present a different shared barcode sequence that serves to identify each such different bead). By way of further example, each nucleic acid probe in a population can include different barcode sequences from all other nucleic acid probes in the population. Alternatively, each nucleic acid probe in a population can include different barcode sequences from some or most other nucleic acid probes in a population. For example, each probe in a population can have a barcode that is present for several different probes in the population even though the probes with the common barcode differ from each other at other sequence regions along their length. In particular embodiments, one or more barcode sequences that are used with a biological specimen (e.g., a tissue sample) are not present in the genome, transcriptome or other nucleic acids of the biological specimen. For example, barcode sequences can have less than 80%, 70%, 60%, 50% or 40% sequence identity to the nucleic acid sequences in a particular biological specimen.

As used herein, “beads”, “microbeads”, “microspheres” or “particles” or grammatical equivalents can include small discrete particles. The composition of the beads can vary, depending upon the class of capture probe, the method of synthesis, and other factors. In certain embodiments of the instant disclosure, the sizes of the beads of the instant disclosure tend to range from 1 μm to 100 μm in diameter (with all subranges within this range expressly contemplated), e.g., depending upon the extent of image resolution desired, nature of the solid support to be used for spatial bead array construction, sequencing processes (e.g., flow cell sequencing) to be employed, as well as other factors.

As used herein, the term “biological specimen” is intended to mean one or more cell, tissue, organism or portion thereof. A biological specimen can be obtained from any of a variety of organisms. Exemplary organisms include, but are not limited to, a mammal such as a rodent, mouse, rat, rabbit, guinea pig, ungulate, horse, sheep, pig, goat, cow, cat, dog, primate (i.e. human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Target nucleic acids can also be derived from a prokaryote such as a bacterium, Escherichia coli, Staphylococci or Mycoplasma pneumoniae; an archae; a virus such as Hepatitis C virus or human immunodeficiency virus; or a viroid. Specimens can be derived from a homogeneous culture or population of the above organisms or alternatively from a collection of several different organisms, for example, in a community or ecosystem.

As used herein, the term “cleavage site” is intended to mean a location in a nucleic acid molecule that is susceptible to bond breakage. The location can be specific to a particular chemical, enzymatic or physical process that results in bond breakage. For example, the location can be a nucleotide that is abasic or a nucleotide that has a base that is susceptible to being removed to create an abasic site. Examples of nucleotides that are susceptible to being removed include uracil and 8-oxo-guanine as set forth in further detail herein below. The location can also be at or near a recognition sequence for a restriction endonuclease such as a nicking enzyme.

By “control” or “reference” is meant a standard of comparison. Methods to select and test control samples are within the ability of those in the art. Determination of statistical significance is within the ability of those skilled in the art, e.g., the number of standard deviations from the mean that constitute a positive result.

As used herein, the term “cryosection” refers to a piece of tissue, e.g. a biopsy, that has been obtained from a subject, snap frozen, embedded in optimal cutting temperature embedding material, frozen, and cut into thin sections. In certain embodiments, the thin sections can be directly applied to an array of beads captured upon a solid support (e.g., a slide), or the thin sections can be fixed (e.g. in methanol or paraformaldehyde) and applied to a bead-presenting planar surface, e.g., a slide upon which a layer of microbeads has been attached/arrayed.

As used herein, the term “different”, when used in reference to nucleic acids, means that the nucleic acids have nucleotide sequences that are not the same as each other. Two or more nucleic acids can have nucleotide sequences that are different along their entire length. Alternatively, two or more nucleic acids can have nucleotide sequences that are different along a substantial portion of their length. For example, two or more nucleic acids can have target nucleotide sequence portions that are different for the two or more molecules while also having a universal sequence portion that is the same on the two or more molecules. Two beads can be different from each other by virtue of being attached to different nucleic acids.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “extend,” when used in reference to a nucleic acid, is intended to mean addition of at least one nucleotide or oligonucleotide to the nucleic acid. In particular embodiments one or more nucleotides can be added to the 3′ end of a nucleic acid, for example, via polymerase catalysis (e.g. DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g. ligase catalysis) methods. A nucleic acid can be extended in a template directed manner, whereby the product of extension is complementary to a template nucleic acid that is hybridized to the nucleic acid that is extended.

As used herein, the term “feature” means a location in an array for a particular species of molecule. A feature can contain only a single molecule or it can contain a population of several molecules of the same species. Features of an array are typically discrete. The discrete features can be contiguous or they can have spaces between each other. The size of the features and/or spacing between the features can vary such that arrays can be high density, medium density or lower density. High density arrays are characterized as having sites separated by less than about 15 μm. Medium density arrays have sites separated by about 15 to 30 μm, while low density arrays have sites separated by greater than 30 μm. An array useful herein can have, for example, sites that are separated by less than 100 μm, 50 μm, 10 μm, 5 μm, 1 μm, or 0.5 μm. An apparatus or method of the present disclosure can be used to detect an array at a resolution sufficient to distinguish sites at the above densities or density ranges.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation.

As used herein, the term “next-generation sequencing” or “NGS” can refer to sequencing technologies that have the capacity to sequence polynucleotides at speeds that were unprecedented using conventional sequencing methods (e.g., standard Sanger or Maxam-Gilbert sequencing methods). These unprecedented speeds are achieved by performing and reading out thousands to millions of sequencing reactions in parallel. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina™); SOLiD™ technology (Applied Biosystems); Ion semiconductor sequencing (Ion Torrent™); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics”, J Genet Genomics, 201, 38(3): 95-109. In certain embodiments, the sequencing parameters of NGS approaches can be modified to allow the instant methods to obtain average read lengths during sequencing (e.g., of TCR sequence-containing cDNAs) of about 200 nucleotides or more, optionally about 250 nucleotides or more, optionally about 300 nucleotides or more, optionally about 350 nucleotides or more, optionally about 400 nucleotides or more, optionally about 450 nucleotides or more, optionally about 500 nucleotides or more. In embodiments, true long read sequencing (LRS) approaches can also be employed to obtain average read lengths that exceed about 500 nucleotides, about 800 nucleotides, about 1000 nucleotides, about 2000 nucleotides, etc., as such approaches can achieve individual read lengths approaching a megabase or more in certain applications, though generally with lower throughput than the above-described NGS methods (as also detailed below). Exemplary forms of long read sequencing include, without limitation, single molecule real time sequencing (SMRT; based on the properties of zero-mode waveguides; signals are in the form of fluorescent light emission from each nucleotide incorporated by a DNA polymerase bound to the bottom of the zL well; developed by PacBio® and used in, e.g., single-cell isoform RNA sequencing (ScISOr-seq)) and nanopore sequencing (which involves passing a DNA molecule through a nanoscale pore structure and then measuring changes in electrical field surrounding the pore, developed by Oxford Nanopore).

As used herein, the terms “nucleic acid” and “nucleotide” are intended to be consistent with their use in the art and to include naturally occurring species or functional analogs thereof. Particularly useful functional analogs of nucleic acids are capable of hybridizing to a nucleic acid in a sequence specific fashion or capable of being used as a template for replication of a particular nucleotide sequence.

Naturally occurring nucleic acids generally have a backbone containing phosphodiester bonds. An analog structure can have an alternate backbone linkage including any of a variety of those known in the art. Naturally occurring nucleic acids generally have a deoxyribose sugar (e.g. found in deoxyribonucleic acid (DNA)) or a ribose sugar (e.g. found in ribonucleic acid (RNA)). A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine, thymine, cytosine or guanine and a ribonucleic acid can have one or more bases selected from the group consisting of uracil, adenine, cytosine or guanine. Useful non-native bases that can be included in a nucleic acid or nucleotide are known in the art. The terms “probe” or “target,” when used in reference to a nucleic acid or sequence of a nucleic acid, are intended as semantic identifiers for the nucleic acid or sequence in the context of a method or composition set forth herein and does not necessarily limit the structure or function of the nucleic acid or sequence beyond what is otherwise explicitly indicated. The terms “probe” and “target” can be similarly applied to other analytes such as proteins, small molecules, cells or the like.

As used herein, the term “poly T or poly A,” when used in reference to a nucleic acid sequence, is intended to mean a series of two or more thiamine (T) or adenine (A) bases, respectively.

A poly T or poly A can include at least about 2, 5, 8, 10, 12, 15, 18, 20 or more of the T or A bases, respectively. Alternatively or additionally, a poly T or poly A can include at most about, 30, 20, 18, 15, 12, 10, 8, 5 or 2 of the T or A bases, respectively.

As used herein, the term “random” can be used to refer to the spatial arrangement or composition of locations on a surface. For example, there are at least two types of order for an array described herein, the first relating to the spacing and relative location of features (also called “sites”) and the second relating to identity or predetermined knowledge of the particular species of molecule that is present at a particular feature. Accordingly, features of an array can be randomly spaced such that nearest neighbor features have variable spacing between each other. Alternatively, the spacing between features can be ordered, for example, forming a regular pattern such as a rectilinear grid or hexagonal grid. In another respect, features of an array can be random with respect to the identity or predetermined knowledge of the species of analyte (e.g., nucleic acid of a particular sequence) that occupies each feature independent of whether spacing produces a random pattern or ordered pattern.

An array set forth herein can be ordered in one respect and random in another. For example, in some embodiments set forth herein a surface is contacted with a population of nucleic acids under conditions where the nucleic acids attach at sites that are ordered with respect to their relative locations but ‘randomly located’ with respect to knowledge of the sequence for the nucleic acid species present at any particular site. Reference to “randomly distributing” nucleic acids at locations on a surface is intended to refer to the absence of knowledge or absence of predetermination regarding which nucleic acid will be captured at which location (regardless of whether the locations are arranged in an ordered pattern or not).

As used herein, the term “solid support” refers to a rigid substrate that is insoluble in aqueous liquid. The substrate can be non-porous or porous. The substrate can optionally be capable of taking up a liquid (e.g. due to porosity) but will typically be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers. Particularly useful solid supports for some embodiments are slides and beads capable of assorting/packing upon the surface of a slide (e.g., beads to which a large number of oligonucleotides are attached).

As used herein, the term “spatial tag” is intended to mean a nucleic acid having a sequence that is indicative of a location. Typically, the nucleic acid is a synthetic molecule having a sequence that is not found in one or more biological specimen that will be used with the nucleic acid. However, in some embodiments the nucleic acid molecule can be naturally derived or the sequence of the nucleic acid can be naturally occurring, for example, in a biological specimen that is used with the nucleic acid. The location indicated by a spatial tag can be a location in or on a biological specimen, in or on a solid support or a combination thereof. A barcode sequence can function as a spatial tag. In certain embodiments, the identification of the tag that serves as a spatial tag is only determined after a population of beads (each possessing a distinct barcode sequence) has been arrayed upon a solid support (optionally randomly arrayed upon a solid support) and sequencing of such a bead-associated barcode sequence has been determined in situ upon the solid support.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the term “tissue” is intended to mean an aggregation of cells, and, optionally, intercellular matter. Typically the cells in a tissue are not free floating in solution and instead are attached to each other to form a multicellular structure. Exemplary tissue types include muscle, nerve, epidermal, connective, lymphatic, and tumor tissues.

As used herein, the term “universal sequence” refers to a series of nucleotides that is common to two or more nucleic acid molecules even if the molecules also have regions of sequence that differ from each other. A universal sequence that is present in different members of a collection of molecules can allow capture of multiple different nucleic acids using a population of universal capture nucleic acids that are complementary to the universal sequence. Similarly, a universal sequence present in different members of a collection of molecules can allow the replication or amplification of multiple different nucleic acids using a population of universal primers that are complementary to the universal sequence. Thus, a universal capture nucleic acid or a universal primer includes a sequence that can hybridize specifically to a universal sequence. Target nucleic acid molecules may be modified to attach universal adapters, for example, at one or both ends of the different target sequences.

Unless specifically stated or obvious from context, as used herein, the term “or” is understood to be inclusive. Unless specifically stated or obvious from context, as used herein, the terms “a”, “an”, and “the” are understood to be singular or plural.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The embodiments set forth below and recited in the claims can be understood in view of the above definitions.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A to 1F show that the foundational “Slide-seq” approach of PCT/US19/30194 enabled RNA capture from tissue with high resolution. FIG. 1A shows a schematic of the method, where DNA barcoded beads were placed onto a rubber surface and barcodes were read out through in situ DNA sequencing. Tissue was then sliced onto the arrays (termed “pucks”) and RNA was transferred in a spatially resolved manner. An RNA sequencing library was then prepared off of the puck and transcripts were linked to spatial locations using the bead barcodes. FIG. 1B at left shows an image of base-calls for one base of sequencing. Inset: blown-up image of base calls for one base of sequencing. Right: Binary image representing connected clusters of pixels all sharing the same barcode, which were then identified as beads. FIG. 1C shows an image of the number of transcripts per bead obtained for a hippocampal puck. FIG. 1D shows characterization of lateral diffusion of signal on the Slide-seq surface. Top Left: Image of a Slide-seq surface with color intensity reflecting transcript counts. Top Right: Image of the adjacent tissue section, stained with DAPI. Boxes represent regions where profile was taken across CA1. (Scale bar: 500 μm). Bottom left: Profile of pixel intensity across CA1 in Slide-seq. Bottom right: Profile across DAPI stained tissue. Red dots represent locations of half max of the distribution. FIG. 1E shows a graph of full width at half maximum of profiles across CA1, as in FIG. 1D, taken across 10 samples. FIG. 1F shows a graph of the number of UMIs captured in the method for the top 1%, 10%, and 20% of beads, for several different tissue types. Error bars indicate the standard deviation across samples.

FIGS. 2A to 2F demonstrate localization of cell types using Slide-seq. FIG. 2A shows a schematic of the method used for assigning cell types to beads using NMF and NNLS regression (NMFreg). FIG. 2B shows spatial locations of beads called as various cerebellar cell types using NMFreg, for one coronal cerebellar puck. Top Left: Raw locations of beads prior to NMFreg. Top Middle: Forty percent of beads called as granular cells by NMFreg are plotted in red. Top right and bottom: the locations of beads assigned to other cell types called by NMFreg, represented as density plots (7). FIG. 2C shows the fraction of beads that can be confidently assigned to different cell types in cerebellar pucks. Error bars represent standard deviation (N=7 cerebellar pucks). FIG. 2D shows the number of beads called as each atlas-defined cell type for cerebellar pucks. Error bars represent standard deviation (N=7 cerebellar pucks). FIG. 2E shows an alignment of serial sections from 66 Slide-seq experiments in the same mouse hippocampus. Cell type calls from NMFreg are projected onto each bead. Green, blue, and red represent beads assigned to the CA1, CA2/3, and dentate gyrus neuron clusters from hippocampal single cell data. The brightness of each sphere is proportional to the number of transcripts on that bead. Top left: View of stack along the medial-lateral axis. Top right: view of stack along the dorsal-ventral axis. Bottom left: individual pucks through the Z plane. The numbers inset indicate distance from the first section in the stack. FIG. 2F shows a density plot of hilum markers (in purple) and CA2 markers (in green) plotted on all beads assigned to clusters 4, 5 and 6 on a cerebellar puck.

FIGS. 3A to 3L show that the foundational Slide-seq approach, now improved upon herein for identification of TCR transcript sequences, identified patterns of spatial gene expression. FIG. 3A shows a coronal cerebellar puck, with Purkinje-assigned beads in blue, choroid-assigned beads in pink, a random subset of other beads in green, and beads expressing Ogfrl1 in red. Bead radius is proportional to the total number of transcripts on the bead (blue and pink) or of Ogfrl1 (red). Red arrow indicates cluster of Ogfrl1-positive beads. FIG. 3B shows an Allen ISH atlas image of Ogfrl1, from a similar brain region, showing expression in the cochlear nucleus. Red arrow indicates Ogfrl1 expression in the cochlear nucleus. FIG. 3C shows the same puck as in FIG. 3A, with beads expressing Rasgrf1 shown in blue, and a subset of other beads in green. FIG. 3D shows an Allen atlas image of Rasgrf1. FIG. 3E shows a heatmap illustrating the separation of Purkinje-expressed genes into two clusters on the basis of the other genes with which they correlate. The i,jth entry is the number of genes found to overlap with both gene i and j in the Purkinje cluster. See Example 1 (Materials and Methods) below. FIG. 3F shows the Aldoc metagene plotted in green, and the Cck metagene plotted in red, both restricted to beads that were called as Purkinje cells. Intensity is proportional to the number of transcripts per bead. FIG. 3G shows the Allen atlas image for Kctd12, in the Aldoc cluster. Red arrow indicates posterior side of lobule V. FIG. 3H shows the Allen atlas image for Cck. FIG. 3I shows the total expression level for each of the indicated metagenes in each of the indicated compartments. The compartments are as shown in FIG. 3G. FIG. 3J shows the correlation between the columns of FIG. 3I. FIGS. 3K and 3L show Allen atlas images of lobule VIII of the cerebellum for the indicated genes. Red arrows indicate the ventral horn of lobule VIII.

FIG. 4 shows a schematic of an exemplary process of the instant disclosure, in which TCR transcript-targeted rhPCR is employed upon a Slide-seq cDNA sample (or a portion thereof) and extended read length sequences capable of resolving individual TCR variable regions are obtained (while also obtaining other identifying sequences within such sequence reads, and optionally while further obtaining other macromolecule abundance data via Slide-seq). At bottom, reconstructed images showing whole transcriptome UMI counts, beads with TRAC or TRBC, and beads with clonotype sequence obtained in performing an exemplary process of the instant disclosure are also shown.

FIG. 5 shows that in initial attempts to resolve TCR sequences using the Slide-Seq process, spatial locations of constant and variable regions did not match up in the human samples examined, which indicated that variable spatial mapping was off.

FIG. 6 shows experiments performed and results obtained involving mixing of human RCC and mouse brain and mouse spleen puck libraries, followed by performance of rhTCR (rhPCR amplification-mediated selective enrichment for TCRs) on such mixed sample(s). The amount of barcode switching was observed to have been very high, as clonotypes that should be human were often seen as mapping to bead barcodes on the mouse pucks. These effects were overcome computationally by testing a few different computational filters, such as >1read/UMI and >1UMI/bead to reduce issues with random mixing. Capture was also improved by pulling the bead and UMI sequences from the constant region sequencing and automatically accepting single reads or UMIs if they matched those sequences. Optionally, emulsion PCR optimization can also be employed to prevent mixing.

FIG. 7 shows data from analyses that employed an improved computational method developed herein. The improved method employed unsupervised clustering, which identified a few regions of interest (lung, immune, tumor). Iterative KNN (k-nearest neighbors) was then performed to assign all remaining unassigned beads to one of those regions. p-values were then calculated to describe how spatially non-random the distribution of different T-cell clonotypes were in space, and it was discovered that several were spatially significant and had different enrichments in the different regions.

### DETAILED DESCRIPTION OF THE INVENTION

The instant disclosure is based, at least in part, upon identification of a method for obtaining spatially-resolvable, high-resolution (at near single-cell resolution) T-cell receptor (TCR) transcript sequence at read lengths that span the TCR variable region, together with spatially-resolvable macromolecule abundance information, directly from a sectioned tissue sample. Previously, TCRs could be sequenced from cells that had been dissociated and processed, either in bulk or single-cell, through the products of whole transcriptome preparations. A significant drawback of such approaches has been their inability to retain spatial information of the T-cells in the tissue, which has been lost through standard whole transcriptome preparations.

The instant disclosure has herein identified that the “Slide-seq” approach initially set forth in PCT/US19/30194—which enabled macromolecule capture (e.g., measurement of transcriptome expression) from sectioned tissues at high spatial resolution—can be successfully adapted to obtain and provide extended length TCR transcript sequences (e.g., TCR transcript sequences that span the TCR variable region, and that optionally include non-variable region TCR transcript sequence such as diversity, joining, constant region and/or transmembrane domain (TMD) sequence) with robust specificity and precision. In particular aspects, the instant disclosure provides for integration into the previously disclosed “Slide-seq” process of PCT/US19/30194 of both (1) RNase H-dependent PCR amplification as a means of targeting TCR transcripts for sequencing with specificity (particularly across the TCR transcript variable region) and (2) extended read length sequencing, either by high-throughput next-generation sequencing (NGS) approaches adapted to obtain extended length sequences or by long read sequencing (LRS). Such extended read length forms of next-generation sequencing (NGS) are capable of producing average read lengths in excess of approximately 200 nucleotides on at least the TCR sequence-containing end of a TCR cDNA or TCR amplicon, and therefore resolving TCR transcript sequences across the entirety of the TCR variable region.

In certain aspects and embodiments, true long read sequencing (LRS, e.g., single molecule real time sequencing (SMRT) or nanopore sequencing) as referenced herein is not required to obtain TCR sequences of sufficient lengths to allow for TCR variable regions to be directly resolved by spanning such regions with individual sequence reads. It is particularly noted that greater throughput than true LRS approaches can often be obtained using standard NGS approaches (e.g., Illumina, Inc. (San Diego, Calif.) MiSeq®, Genome Analyzer®, NextSeq®, HiSeq®, etc. platforms), yet with parameters adjusted to allow for longer read lengths to be obtained by such methods. Certain aspects of the “Slide-seq” approach set forth in PCT/US19/30194 disclose generation of cDNA libraries that are cleaved and fragmented (“tagmented”) before sequencing such that the ultimate sequencing readout only contains the 3′ end of whatever was captured. Resolution of TCR sequences using such approaches has heretofore posed a challenge, because TCR transcript variable regions of interest are positioned too far away from the 3′ end of captured sequences in such approaches for the TCR transcript variable regions to be spanned with reads of sufficient length to allow for the TCR transcript variable regions to be resolved directly (by knowing the sequence of each individual TCR transcript sequence read at a length that encompasses relevant regions of TCR transcript variable regions, as opposed to certain previously disclosed approaches that infer read identities via statistical/informatics approaches performed upon populations of sequence).

Accordingly, in certain aspects, the instant disclosure addresses the need for obtaining extended length TCR transcript sequences for purpose of TCR transcript variable region resolution within individual reads in the following manner. Before fragmentation, a TCR-targeted amplification using RNase H-dependent PCR (rhPCR) is performed upon a cDNA library (or portion thereof) generated by the “Slide-seq” approach. Using standard NGS approaches, sequencing parameters are then adjusted to obtain longer individual sequence read lengths—for example, when using an Illumina, Inc. (San Diego, Calif.) MiSeq® platform in the instant Examples, sequencing parameters were adjusted to obtain a much longer read on Read2, allowing for spanning and resolution of the TCR transcript variable regions in individual reads. Notably, is not a method that is traditionally used to amplify DNA. Identification and integration of rhPCR into the Slide-seq approach set forth in PCT/US19/30194, as now disclosed herein, therefore provides a non-obvious advance over the original Slide-seq approach for obtainment of TCR transcript sequences. While true long read sequencing (LRS) can be employed in certain embodiments of the instant disclosure, simply performing LRS upon cDNA populations in the absence of rhPCR represents a highly inefficient way of obtaining TCR transcript sequences of sufficient length to span and resolve the TCR transcript variable region, as many of the TCR transcript sequences are lowly expressed and would therefore constitute only a small fraction of any sequencing that hasn't been enriched. Performance of rhPCR as provided for in the processes of the instant disclosure is therefore significantly more efficient than simply performing LRS directly on the Slide-seq cDNA library in the absence of such TCR-specific rhPCR amplification.

In certain embodiments, a cDNA library obtained via the initial steps of the “Slide-seq” process is split before being cleaved and tagged (“tagmented”) in preparation for sequencing, with a portion of the split cDNA population subjected to RNase H-dependent PCR amplification (e.g., as disclosed in Li et al. Nat. Protoc. 14: 2571-2594). RNase H-dependent PCR amplification provides for amplification of targeted TCR transcripts across V, (D), J and C segments with enhanced specificity via use of amplification primers possessing a blocked 3′ end and an internal RNA that is cleaved and removed by RNase H only when a highly specific annealing (high fidelity annealing) of an amplification primer to a target sequence has occurred. Employment of rhPCR primers specifically directed to TCR transcripts therefore provides for clean TCR transcript sequences of sufficient length to span and resolve the TCR transcript variable region to be obtained, and for identification of paired TCR-α and TCR-β sequences.

Each newly-differentiated T or B lymphocyte in the immune system carries a different antigen receptor as the result of critical DNA rearrangements that alter the 450 nucleotides at the 5′ end of their T- or B-cell antigen-receptor mRNA (Redmond et al. Genome Medicine 8, Article number: 80; Dash et al. J. Clin. Invest. 121: 288-95). Because T cell rearrangements occur at such distances, to obtain extended length and/or paired TCR-α and TCR-β sequences at near single-cell resolution (without reliance upon statistical methods that model such rearrangements within populations of TCR sequences), it is highly preferred to use sequencing methods upon individual TCR transcripts that are capable of obtaining longer average read lengths than the most commonly used next-generation sequencing (NGS) methods. Accordingly, in certain embodiments, sequencing methods that provide an average read length on at least one end of a TCR sequence-containing cDNA/PCR amplicon of at minimum 200 nucleotides of continuous sequence are employed herein (via adaptation of NGS methods to obtain extended read lengths). By employing and/or adapting sequencing methods to achieve longer read lengths, the variable region of the TCR can be traversed.

In certain aspects, the instant disclosure provides methods for obtaining not only spatially-localizable extended length TCR transcript sequences but also spatially-localizable macromolecule abundance data (e.g., expression and/or transcriptome data, tagged protein abundance information, etc.), via employment of the previously described “Slide-seq” process as adapted herein to allow for robust extended length TCR transcript identification and assessment. Using the approaches disclosed herein, TCR transcript sequences (including TCR variable sequences) and macromolecule abundance data can be obtained in parallel and optionally overlaid or otherwise compared, reported or profiled in space.

Accordingly, contemplated advantages of the methods disclosed herein include, without limitation, (1) providing a means for sequencing TCRs and reporting their original locations in an assayed/sectioned tissue, alongside that of other cells in space and (2) higher efficiency capture per bead than shown previously for the “Slide-seq” approach (at least because most TCR transcripts are relatively lowly expressed).

It is further explicitly contemplated, without limitation, that the approaches of the instant disclosure can be applied to study T-cell receptor sequences in various tissues. The methods of the instant disclosure are readily amenable to different tissue inputs. In particular, among other applications, the instant methods can be used to examine T-cell development in lymphoid organs, as well as whether or not certain TCRs possess improved/poor behavior in pathogenesis (e.g. if certain TCRs can penetrate certain tumor types better than others).

Various expressly contemplated components of certain compositions and methods of the instant disclosure are considered in additional detail below.

### T-Cell Receptor Transcript Sequences and Transcriptome Analysis

Various aspects disclosed herein provide methods for obtaining spatially-resolvable sequencing of TCR transcripts. Antigen-specific T cells play key roles in a number of diseases including autoimmune disorders and cancer (Schrama et al. Semin. Immunopathol. 39: 255-268; Lossius A et al. Eur. J. Immunol. 44: 1-41; Kirsch I R et al. Sci. Transl. Med 7: 1-13). Assessing the phenotypes and functions of these cells has been described as essential to both understanding underlying disease biology and designing new therapeutic modalities (Carlson C S et al. Nat. Commun 4: 2680; Crosby E J et al. Oncoimmunology 7: e1421891). To study antigen-specific T cells comprehensively, two sequencing-based approaches have emerged: bulk genomic sequencing of T cell antigen receptor (TCR) gene repertoires to assess clonal diversity; and RNA-sequencing (RNA-seq) to reveal phenotypic attributes. The TCR recognizes antigenic peptides bound in major histocompatibility complex (MHC) receptors and mediates CD3-dependent signaling upon cognate recognition; sequencing of the TCR repertoire thus can highlight clonotypic diversity and the dynamics of antigen-dependent responses associated with disease, such as clonal expansion or selection (Lossius A et al. Eur. J. Immunol. 44: 1-41; Tirosh I et al. Science 352: 189-196; Khodadoust M S et al. Nature 543: 723-727). RNA-seq, in contrast, can reveal novel states and functions of disease-relevant T cells through unique patterns of gene expression, albeit without determination of whether those cells are recognizing common antigens (Avraham R et al. Cell 162: 1309-1321; Papalexi E & Satija R. Nat. Rev. Immunol 18: 35-45; Shalek A K et al. Nature 498: 236-240).

Tu et al. (Nat. Immunol. 20: 1692-1699) recently described a process for obtaining sequence concomitantly of both the transcriptome of T cells and of TCR sequences of T cells, from a single sequencing library generated using a massively parallel 3′ scRNA-seq platform, such as Seq-Well or Drop-seq. However, a need has existed in the art for an approach that is capable of concomitantly obtaining and assessing both the transcriptome of cells (e.g., T-cells) and of TCR sequences of T cells, in a manner that is spatially-resolvable at high resolution and at sufficient sequencing depth. In certain aspects, the methods disclosed herein address this need.

### RNase H-Dependent PCR for TCR Sequencing

Certain aspects of the instant disclosure employ RNase H-dependent PCR (rhPCR) amplification as a means of selectively amplifying TCR transcripts in a manner that obtains identifiable extended length sequences of individual TCR transcripts (each also carrying a spatially-resolvable identification sequence). rhPCR uses 3′-blocked oligonucleotides with a single ribo residue located approximately five nucleotides from the 3′ end. By including thermostable RNase H in the amplification reaction, these blocked oligonucleotides are cleaved at the RNA base if, and only if, the oligonucleotide is hybridized to an appropriate target. Cleavage generates a free 3′-hydroxyl that is extended by Taq DNA polymerase. Thus, functional primers are generated in situ during the PCR and accurate hybridization of the proto-primers is required during every round of PCR in order to achieve exponential amplification. This technique is very specific because the absence of free primers not hybridized to target essentially eliminates primer dimer formation, and the requirement of RNase H for high-fidelity base pairing severely reduces off-target amplification (Li et al. Nat. Protoc. 14: 2571-2594).

### Next-Generation Sequencing (NGS) Approaches Possessing Long Average Read Lengths

In some aspects, the improved methods of the instant disclosure employ next-generation sequencing (NGS) approaches that are designed and/or adapted to provide extended read lengths, particularly for TCR sequence-containing ends of cDNA-derived nucleic acid fragments that are sequenced, thereby allowing for TCR variable regions to be resolved and clonotypes to be identified discretely (e.g., average read lengths for at least one end of cDNA-derived sequences exceeding 200 nucleotides in length are obtained, optionally with average fragment read lengths exceeding 250 nucleotides in length, etc., optionally for only the TCR sequence-containing end of cDNA-derived nucleic acids).

NGS, as defined above, has dominated the DNA sequencing space since its development. It has dramatically reduced the cost of DNA sequencing by enabling a massively-paralleled approach capable of producing large numbers of reads at exceptionally high coverages throughout the genome (Treangen and Salzberg. Nature Reviews Genetics 13: 36-46).

NGS works by first amplifying the DNA molecule and then conducting sequencing by synthesis. The collective fluorescent signal resulting from synthesizing a large number of amplified identical DNA strands allows the inference of nucleotide identity. However, due to random errors, DNA synthesis between the amplified DNA strands would become progressively out-of-sync. Quickly, the signal quality deteriorates as the read-length grows. In order to preserve read quality, long DNA molecules must be broken up into small segments, resulting in a critical limitation of NGS technologies (Treangen and Salzberg). Computational efforts aimed to overcome this challenge often rely on approximative heuristics that may not result in accurate assemblies.

It is noted that long-read sequencing (LRS) technologies offer improvements in the characterization of genetic variation and regions that are difficult to assess with prevailing NGS approaches. Long-Read Sequencing (LRS) is a class of DNA sequencing methods currently under active development (Bleidorn, Christoph. Systematics and Biodiversity 14: 1-8). Long-read sequencing works by reading the nucleotide sequences at the single molecule level, in contrast to existing methods that require breaking long strands of DNA into small segments then inferring nucleotide sequences by amplification and synthesis (“Illumina sequencing technology” PDF). By enabling direct sequencing of single DNA molecules, long-read sequencing (LRS) technologies have the capability to produce substantially longer reads than second generation sequencing (Bleidorn). Such an advantage has critical implications for both genome science and the study of biology in general. However, long-read sequencing data have exhibited much higher error rates than previous technologies, which can complicate downstream genome assembly and analysis of the resulting data (Gupta. Trends in Biotechnology 26: 602-611). These technologies are undergoing active development and it is expected that there will be improvements to the high error rates. For applications that are more tolerant to error rates, such as structural variant calling, long-read sequencing has been found to outperform existing methods. As noted above, however, to date, the throughput obtained using true LRS approaches has also been less than for standard NGS approaches. Thus, in currently preferred embodiments standard NGS approaches (adapted to obtain extended read lengths from at least one end of sequenced nucleic acid fragments) are used to resolve TCR variable sequence-containing ends of sequenced nucleic acids.

Several companies are currently at the heart of long-read sequencing technology development, namely, Pacific Biosciences, Oxford Nanopore Technology, Quantapore (CA-USA), and Stratos (WA-USA). These companies are taking fundamentally different approaches to sequencing single DNA molecules.

PacBio® developed the sequencing platform of single molecule real time sequencing (SMRT), based on the properties of zero-mode waveguides. Signals are in the form of fluorescent light emission from each nucleotide incorporated by a DNA polymerase bound to the bottom of the zL well. A current example of a PacBio® long-read sequencing platform employed herein is ScISOr-seq.

Oxford Nanopore's technology involves passing a DNA molecule through a nanoscale pore structure and then measuring changes in electrical field surrounding the pore; while Quantapore has a different proprietary nanopore approach. Stratos Genomics spaces out the DNA bases with polymeric inserts, “Xpandomers”, to circumvent the signal to noise challenge of nanopore ssDNA reading. R2C2 (Rolling Circle Amplification to Concatemeric Consensus) is noted as an exemplary Nanopore isoform sequencing method.

In certain embodiments, nanopore sequencing is employed (see, e.g., Astier et al, J. Am. Chem. Soc. 2006 Feb. 8; 128(5): 1705-10, which is incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore (or as individual nucleotides pass through the nanopore in the case of exonuclease-based techniques), this causes a change in the magnitude of the current through the nanopore that is distinct for each of the four bases, thereby allowing the sequence of the DNA molecule to be determined.

For optimizing implementation of LRS, it is further contemplated that rhPCR-amplified TCR sequences possessing spatially-resolvable identifiers (optionally together with broader transcriptome sequences and/or other tagged macromolecules) can be subjected to Chimeric Array Sequencing (CAseq) methods as described in U.S. Ser. No. 62/933,794 CAseq was specifically identified as capable of increasing throughput of long-read sequencing platforms by >10× while also decreasing sequencing artifacts by >90%. The CAseq method is a specialized multiplexing workflow that boosts molecular sequencing output of long-read sequencers by catering to the unique characteristics of these platforms. In contrast to Illumina®'s short-read sequencing workflows, which have specified read lengths, long-read platforms have indeterminate read lengths that can range from ˜20 kb up to a staggering 2 Mb per pore (MinION, Oxford Nanopore Technologies) or well (Sequel II, PacBio®) in a flowcell. These massive read lengths are optimal for efforts such as bulk whole genome sequencing, but prior to development of CAseq, seemed excessive for intermediate length targets (500 bp-10 kb) such as extended length transcripts, particularly the TCR transcripts that are a focus of the instant disclosure. It is therefore contemplated that the methods of the instant disclosure can employ assemblies of chimeric arrays as described in U.S. Ser. No. 62/933,794 to achieve optimal yield of useful extended length transcript information from populations of intermediate length target molecules (e.g., TCR transcripts, the broader transcriptome and/or nucleic acid tags of associated macromolecules such as antibodies).

Paired-End Sequencing for Identification of Bead Identification Sequences Associated with TCR Sequences and/or Macromolecules

Certain aspects of the instant disclosure also employ NGS methods that do not require extended read lengths, e.g., to obtain bead identification sequences associated with individual TCR sequences and/or individual macromolecules within a sequenced population. Such bead identification sequences (or oligonucleotide cluster and/or array identification sequences) therefore render the TCR sequences and/or macromolecule abundance information spatially-resolvable. It is expressly contemplated that paired-end sequencing can be performed upon nucleic acid populations of the instant disclosure to obtain such identifiers and associated macromolecules/transcripts. Paired-end sequencing is known in the art, with exemplary description found in, e.g., Fullwood et al., “Next-generation DNA sequencing of paired-end tags (PET) for transcriptome and genome analyses” Genome Res. 19:521-532 (2009), US 2014/0031241, EP Patent No. 2,084,295 and U.S. Pat. No. 7,601,499.

### Slide-Seq Platform

Certain aspects of the instant disclosure expand upon the original “Slide-seq” technology platform of PCT/US19/30194, specifically employing the same beads, arrays and sequencing chemistry as “Slide-seq”.

In certain aspects relevant to the instant disclosure, “Slide-seq” refers to a tightly packed spatially barcoded microbead array (e.g., an array of 10 μm diameter beads packed at an inter-bead spacing of 20 μm or less, where each bead possesses a bead-specific barcode within bead-attached capture oligonucleotides) created via application of a capture material to a solid support (e.g., application of a liquid electrical tape to a glass slide, followed by application of a layer of microbeads) that can be used to capture cellular transcriptomes (or other macromolecules) of sectioned tissue (optionally, cryosectioned tissue), in a manner that is both spatially resolvable at high resolution (e.g., at resolutions of 20 μm between image features) and with deep coverage (i.e., high-resolution images of relative expression for individual transcripts can be generated using the methods and compositions of the instant disclosure, for a large number (i.e., tens, hundreds or even thousands) of transcripts, across an individual sectioned tissue sample).

“Slide-seq” enables spatially resolved capture of nucleic acids for sequencing from cells and tissues with approximate 10 μm (single cell) resolution. Pre-“Slide-seq” spatial profiling technologies have relied upon either targeted in situ techniques, which were laborious and offered only a low degree of multiplexing with a high degree of technical difficulty, or have offered only very low resolution on spatial capture arrays (resolutions of approximately 100-200 μm). “Slide-seq” provides a level of image resolution that is a full order of magnitude superior in lateral resolution, and two orders of magnitude superior in capture area. By using mRNA capture and subsequent high-throughput sequencing (e.g., by Illumina™ bead-based sequencing), spatially-localizable whole transcriptomic profiling of complex tissues can be performed.

Certain aspects of “Slide-seq” employ a spatially barcoded array of oligonucleotide-laden beads to capture mRNA from tissue sections. Exemplified beads are synthesized with a unique or sufficiently unique bead barcode as previously described, e.g., in WO 2016/040476 (PCT/US2015/049178), wherein an exemplary sufficiently unique bead barcode is one that is a member of a population of barcode sequences that is sufficiently degenerate to a population (e.g., of beads) that a majority of individual components (e.g. beads) of the barcoded population each possesses a unique barcode sequence, where the remainder (minority) of the population may possess barcodes that are redundant with those of other members within the remainder population, yet such redundancy can either be eliminated or otherwise adjusted for (e.g., normalized, averaged across/between redundant members, etc.) with only minor impact upon, e.g., the image resolution obtained when employing such a barcoded population. “Slide-seq” specifically provides for: 1) tiling of beads into a monolayer surface; 2) interrogation of the sequence of each bead barcode of the surface via sequencing by ligation on an standard microscope; 3) capture of RNA from cells and tissues onto the bead array, particularly noting the instant use of sectioned tissue samples; 4) performing reverse transcription (RT) and generating barcoded sequencing libraries as previously described in WO 2016/040476; and 5) next-generation sequencing of the barcoded libraries (exemplified herein using an Illumina™ platform) followed by bead barcode matching to the spatial location of the read. Generation of high-resolution barcoded arrays via on-surface sequencing of capture probe beads (noting that exemplified beads have been prepared as previously described in WO 2016/040476) was a distinguishing feature of “Slide-seq”, as well as techniques to capture RNA to the barcoded bead array.

The “Slide-seq” approach therefore enabled the localization of cell types and gene expression patterns in tissue with 10-micron resolution in an unbiased manner.

The Slide-seq approach provided a method that was demonstrated to enable facile generation of large volumes of unbiased spatial transcriptomes with 10 μm spatial resolution, comparable to the size of individual cells. To perform Slide-seq, RNA is transferred from freshly frozen tissue sections onto a surface covered in DNA-barcoded polystyrene beads with known positions. Subsequent sequencing of the bead-anchored RNA allows for the assignment of beads to known cell types derived from scRNAseq data, revealing the spatial organization of cell types in the tissue with 10 μm resolution. Slide-seq was initially applied to systematically characterize spatial gene expression patterns in the Purkinje layer of the mouse cerebellum, identifying several genes not previously associated with Purkinje cell compartments. Applying Slide-seq to a model of traumatic brain injury further allowed for the characterization of underlying genetic programs varying over time and space in response to injury. Slide-seq has thus provided a new methodology to identify novel molecular patterns within tissues at high resolution and can accommodate large volumes of tissue, thereby enabling the generation of high resolution transcriptome atlases at scale, among other applications.

### Solid Supports

In certain aspects, the present disclosure employs a spatially tagged array of microbeads to perform deep expression profiling upon sectioned tissue samples, with high image resolution. Methods can include the steps of (a) attaching different nucleic acid probes to beads that are then captured upon a solid support to produce randomly located probe-possessing beads on the solid support, wherein the different nucleic acid probes each includes a barcode sequence (that is shared by all such nucleic acid probes of a single bead), and wherein each of the randomly located beads includes a different barcode sequence(s) from other randomly located beads on the solid support; (b) performing a nucleic acid detection reaction on the solid support to determine the barcode sequences of the randomly located beads on the solid support; (c) contacting a biological specimen with the solid support that has the randomly located beads; (d) hybridizing the probes presented by the randomly located beads to target nucleic acids from portions of the biological specimen that are proximal to the randomly located beads; and (e) extending the probes of the randomly located beads to produce extended probes that include the barcode sequences and sequences from the target nucleic acids, thereby spatially tagging the nucleic acids of the biological specimen.

Any of a variety of solid supports can be used in a method, composition or apparatus of the present disclosure. Particularly useful solid supports are those used for nucleic acid arrays. Examples include glass, modified glass, functionalized glass, inorganic glasses, microspheres (e.g. inert and/or magnetic particles), plastics, polysaccharides, nylon, nitrocellulose, ceramics, resins, silica, silica-based materials, carbon, metals, an optical fiber or optical fiber bundles, polymers and multiwell (e.g. microtiter) plates. Exemplary plastics include acrylics, polystyrene, copolymers of styrene and other materials, polypropylene, polyethylene, polybutylene, polyurethanes and Teflon™. Exemplary silica-based materials include silicon and various forms of modified silicon.

In particular embodiments, a solid support can be within or part of a vessel such as a well, tube, channel, cuvette, Petri plate, bottle or the like. Optionally, the vessel is a flow-cell, for example, as described in WO 2014/142841 A1; U.S. Pat. App. Pub. No. 2010/0111768 A1 and U.S. Pat. No. 8,951,781 or Bentley et al., Nature 456:53-59 (2008), each of which is incorporated herein by reference. Exemplary flow-cells are those that are commercially available from Illumina, Inc. (San Diego, Calif.) for use with a sequencing platform such as a Genome Analyzer®, MiSeq®, NextSeq® or HiSeq® platform. Optionally, the vessel is a well in a multiwell plate or microtiter plate.

In certain embodiments, a solid support can include a gel coating. Attachment, e.g., of nucleic acids to a solid support via a gel is exemplified by flow cells available commercially from Illumina Inc. (San Diego, Calif.) or described in US Pat. App. Pub. Nos. 2011/0059865 A1, 2014/0079923 A1, or 2015/0005447 A1; or PCT Publ. No. WO 2008/093098, each of which is incorporated herein by reference. Exemplary gels that can be used in the methods and apparatus set forth herein include, but are not limited to, those having a colloidal structure, such as agarose; polymer mesh structure, such as gelatin; or cross-linked polymer structure, such as polyacrylamide, SFA (see, for example, US Pat. App. Pub. No. 2011/0059865 A1, which is incorporated herein by reference) or PAZAM (see, for example, US Pat. App. Publ. Nos. 2014/0079923 A1, or 2015/0005447 A1, each of which is incorporated herein by reference).

In some embodiments, a solid support can be configured as an array of features to which beads can be attached. The features can be present in any of a variety of desired formats. For example, the features can be wells, pits, channels, ridges, raised regions, pegs, posts or the like. Exemplary features include wells that are present in substrates used for commercial sequencing platforms sold by 454 LifeSciences (a subsidiary of Roche, Basel Switzerland) or Ion Torrent (a subsidiary of Life Technologies, Carlsbad Calif.). Other substrates having wells include, for example, etched fiber optics and other substrates described in U.S. Pat. Nos. 6,266,459; 6,355,431; 6,770,441; 6,859,570; 6,210,891; 6,258,568; 6,274,320; US Pat app. Publ. Nos. 2009/0026082 A1; 2009/0127589 A1; 2010/0137143 A1; 2010/0282617 A1 or PCT Publication No. WO 00/63437, each of which is incorporated herein by reference. In some embodiments, wells of a substrate can include gel material (with or without beads) as set forth in US Pat. App. Publ. No. 2014/0243224 A1, which is incorporated herein by reference.

Features can appear on a solid support as a grid of spots or patches. The features can be located in a repeating pattern or in an irregular, non-repeating pattern. Optionally, repeating patterns can include hexagonal patterns, rectilinear patterns, grid patterns, patterns having reflective symmetry, patterns having rotational symmetry, or the like. Asymmetric patterns can also be useful.

The pitch of an array can be the same between different pairs of nearest neighbor features or the pitch can vary between different pairs of nearest neighbor features.

In particular embodiments, features on a solid support can each have an area that is larger than about 100 nm², 250 nm², 500 nm², 1 μm², 2.5 μm², 5 μm², 10 μm² or 50 μm². Alternatively or additionally, features can each have an area that is smaller than about 50 μm², 25 μm², 10 μm², 5 μm², 1 μm², 500 nm², or 100 nm². The preceding ranges can describe the apparent area of a bead or other particle on a solid support when viewed or imaged from above.

Beads

Certain aspects of the instant disclosure employ a collection of beads or other particles, to which oligonucleotides are attached. Suitable bead compositions include those used in peptide, nucleic acid and organic moiety synthesis, including, but not limited to, plastics, ceramics, glass, polystyrene, methylstyrene, acrylic polymers, paramagnetic materials, thoriasol, carbon graphite, titanium dioxide, latex or cross-linked dextrans such as Sepharose, cellulose, nylon, cross-linked micelles and Teflon may all be used. “Microsphere Detection Guide” from Bangs Laboratories, Fishers Ind. is a helpful guide, which is incorporated herein by reference in its entirety. The beads need not be spherical; irregular particles may be used. In addition, the beads may be porous, thus increasing the surface area of the bead available for either capture probe attachment or tag attachment. The bead sizes can range from nanometers, for example, 100 nm, to millimeters, for example, 1 mm, with beads from about 0.2 μm to about 200 μm commonly employed, and from about 5 to about 20 μm being within the range currently exemplified, although in some embodiments smaller or larger beads may be used.

The particles can be suspended in a solution or they can be located on the surface of a substrate (e.g., arrayed upon the surface of a solid support, such as a glass slide). Art-recognized examples of arrays having beads located on a surface include those wherein beads are located in wells such as a BeadChip array (Illumina Inc., San Diego Calif.), substrates used in sequencing platforms from 454 LifeSciences (a subsidiary of Roche, Basel Switzerland) or substrates used in sequencing platforms from Ion Torrent (a subsidiary of Life Technologies, Carlsbad Calif.). Other solid supports having beads located on a surface are described in U.S. Pat. Nos. 6,266,459; 6,355,431; 6,770,441; 6,859,570; 6,210,891; 6,258,568; or 6,274,320; US Pat. App. Publ. Nos. 2009/0026082 A1; 2009/0127589 A1; 2010/0137143 A1; or 2010/0282617 A1 or PCT Publication No. WO 00/63437, each of which is incorporated herein by reference. Several of the above references describe methods for attaching nucleic acid probes to beads prior to loading the beads in or on a solid support. As such, the collection of beads can include different beads each having a unique (or sufficiently unique and/or near-unique, as described elsewhere herein) probe attached. It will however, be understood that the beads can be made to include universal primers, and the beads can then be loaded onto an array, thereby forming universal arrays for use in a method set forth herein. The solid supports typically used for bead arrays can be used without beads. For example, nucleic acids, such as probes or primers can be attached directly to the wells or to gel material in wells. Thus, the above references are illustrative of materials, compositions or apparatus that can be modified for use in the methods and compositions set forth herein.

Accordingly, the instant methods can employ an array of beads, wherein different nucleic acid probes are attached to different beads in the array. In this embodiment, each bead can be attached to a different nucleic acid probe and the beads can be randomly distributed on the solid support in order to effectively attach the different nucleic acid probes to the solid support. Optionally, the solid support can include wells having dimensions that accommodate no more than a single bead. In such a configuration, the beads may be attached to the wells due to forces resulting from the fit of the beads in the wells. As described elsewhere herein, it is also possible to use attachment chemistries or capture materials (e.g., liquid electrical tape) to adhere or otherwise stably associate the beads with a solid support, optionally including holding the beads in wells that may or may not be present on a solid support.

Nucleic acid probes that are attached to beads can include barcode sequences. A population of the beads can be configured such that each bead is attached to only one type of barcode (e.g., a spatial barcode) and many different beads each with a different barcode are present in the population. In this embodiment, randomly distributing the beads to a solid support will result in randomly locating the nucleic acid probe-presenting beads (and their respective barcode sequences) on the solid support. In some cases, there can be multiple beads with the same barcode sequence such that there is redundancy in the population. However, randomly distributing a redundancy-comprising population of beads on a solid support—especially one that has a capacity that is greater than the number of unique barcodes in the bead population—will tend to result in redundancy of barcodes on the solid support, which will tend to reduce image resolution in the context of the instant disclosure (i.e., where the precise location of a barcoded bead cannot be resolved due to redundancy of barcode use within an arrayed population of beads, it is contemplated that such redundant locations will simply be eliminated from an ultimate image produced by methods of the instant disclosure, or other modes of adjustment (e.g., normalization and/or averaging of values) may also be employed to address such redundancies). Alternatively, in preferred embodiments, the number of different barcodes in a population of beads can exceed the capacity of the solid support in order to produce an array that is not redundant with respect to the population of barcodes on the solid support. The capacity of the solid support will be determined in some embodiments by the number of features (e.g. single-bead occupancy wells) that attach or otherwise accommodate a bead.

A bead or other nucleic acid-presenting solid support of the instant disclosure can include, or can be made by the methods set forth herein to attach, a plurality of different nucleic acid probes.

For example, a bead or other nucleic acid-presenting solid support can include at least 10, 100, 1×10³, 1×10⁴, 1×10⁵, 1×10⁶, 1×10⁷, 1×10⁸, 1×10⁹ or more different probes. Alternatively or additionally, a bead or other nucleic acid-presenting solid support can include at most 1×10⁹, 1×10⁸, 1×10⁷, 1×10⁶, 1×10⁵, 1×10⁴, 1×10³, 100, or fewer different probes. It will be understood that each of the different probes can be present in several copies, for example, when the probes have been amplified to form a cluster. Thus, the above ranges can describe the number of different nucleic acid clusters on a bead or other nucleic acid-presenting solid support of the instant disclosure. It will also be understood that the above ranges can describe the number of different barcodes, target capture sequences, or other sequence elements set forth herein as being unique (or sufficiently unique) to particular nucleic acid probes. Alternatively or additionally, the ranges can describe the number of extended probes or modified probes created on a bead or other nucleic acid-presenting solid support of the instant disclosure using a method set forth herein.

Features may be present on a bead or other solid support of the instant disclosure prior to contacting the bead or other solid support with nucleic acid probes. For example, in embodiments where probes are attached to a bead or other solid support via hybridization to primers, the primers can be attached at the features, whereas interstitial areas outside of the features substantially lack any of the primers. Nucleic acid probes can be captured at preformed features on a bead or other solid support, and optionally amplified on the bead or other solid support, e.g., using methods set forth in U.S. Pat. Nos. 8,895,249 and 8,778,849 and/or U.S. Patent Publication No. 2014/0243224 A1, each of which is incorporated herein by reference. Alternatively, a bead or other solid support may have a lawn of primers or may otherwise lack features. In this case, a feature can be formed by virtue of attachment of a nucleic acid probe on the bead or other solid support. Optionally, the captured nucleic acid probe can be amplified on the bead or other solid support such that the resulting cluster becomes a feature. Although attachment is exemplified above as capture between a primer and a complementary portion of a probe, it will be understood that capture moieties other than primers can be present at pre-formed features or as a lawn. Other exemplary capture moieties include, but are not limited to, chemical moieties capable of reacting with a nucleic acid probe to create a covalent bond or receptors capable of binding non-covalently to a ligand on a nucleic acid probe.

A step of attaching nucleic acid probes to a bead or other solid support can be carried out by providing a fluid that contains a mixture of different nucleic acid probes and contacting this fluidic mixture with the bead or other solid support. The contact can result in the fluidic mixture being in contact with a surface to which many different nucleic acid probes from the fluidic mixture will attach. Thus, the probes have random access to the surface (whether the surface has pre-formed features configured to attach the probes or a uniform surface configured for attachment). Accordingly, the probes can be randomly located on the bead or other solid support.

The total number and variety of different probes that end up attached to a surface can be selected for a particular application or use. For example, in embodiments where a fluidic mixture of different nucleic acid probes is contacted with a bead or other solid support for purposes of attaching the probes to the support, the number of different probe species can exceed the occupancy of the bead or other solid support for probes. Thus, the number and variety of different probes that attach to the bead or other solid support can be equivalent to the probe occupancy of the bead or other solid support.

Alternatively, the number and variety of different probe species on the bead or other solid support can be less than the occupancy (i.e. there will be redundancy of probe species such that the bead or other solid support may contain multiple features having the same probe species). Such redundancy can be achieved, for example, by contacting the bead or other solid support with a fluidic mixture that contains a number and variety of probe species that is substantially lower than the probe occupancy of the bead or other solid support.

Attachment of the nucleic acid probes can be mediated by hybridization of the nucleic acid probes to complementary primers that are attached to the bead or other solid support, chemical bond formation between a reactive moiety on the nucleic acid probe and the bead or other solid support (examples are set forth in U.S. Pat. Nos. 8,895,249 and 8,778,849, and in U.S. Patent Publication No. 2014/0243224 A1, each of which is incorporated herein by reference), affinity interactions of a moiety on the nucleic acid probe with a bead- or other solid support-bound moiety (e.g. between known receptor-ligand pairs such as streptavidin-biotin, antibody-epitope, lectin-carbohydrate and the like), physical interactions of the nucleic acid probes with the bead or other solid support (e.g. hydrogen bonding, ionic forces, van der Waals forces and the like), or other interactions known in the art to attach nucleic acids to surfaces.

In some embodiments, attachment of a nucleic acid probe is non-specific with regard to any sequence differences between the nucleic acid probe and other nucleic acid probes that are or will be attached to the bead or other solid support. For example, different probes can have a universal sequence that complements surface-attached primers or the different probes can have a common moiety that mediates attachment to the surface. Alternatively, each of the different probes (or a subpopulation of different probes) can have a unique (or sufficiently unique) sequence that complements a unique (or sufficiently unique) primer on the bead or other solid support or they can have a unique (or sufficiently unique) moiety that interacts with one or more different reactive moiety on the bead or other solid support. In such cases, the unique (or sufficiently unique) primers or unique (or sufficiently unique) moieties can, optionally, be attached at predefined locations in order to selectively capture particular probes, or particular types of probes, at the respective predefined locations.

One or more features on a bead or other solid support can each include a single molecule of a particular probe. The features can be configured, in some embodiments, to accommodate no more than a single nucleic acid probe molecule. However, whether or not the feature can accommodate more than one nucleic acid probe molecule, the feature may nonetheless include no more than a single nucleic acid probe molecule. Alternatively, an individual feature can include a plurality of nucleic acid probe molecules, for example, an ensemble of nucleic acid probe molecules having the same sequence as each other. In particular embodiments, the ensemble can be produced by amplification from a single nucleic acid probe template to produce amplicons, for example, as a cluster attached to the surface.

A method set forth herein can use any of a variety of amplification techniques. Exemplary techniques that can be used include, but are not limited to, polymerase chain reaction (PCR), rolling circle amplification (RCA), multiple displacement amplification (MDA), or random prime amplification (RPA). In some embodiments the amplification can be carried out in solution, for example, when features of an array are capable of containing amplicons in a volume having a desired capacity. In certain embodiments, an amplification technique used in a method of the present disclosure will be carried out on solid phase. For example, one or more primer species (e.g. universal primers for one or more universal primer binding site present in a nucleic acid probe) can be attached to a bead or other solid support. In PCR embodiments, one or both of the primers used for amplification can be attached to a bead or other solid support (e.g. via a gel). Formats that utilize two species of primers attached to a bead or other solid support are often referred to as bridge amplification because double stranded amplicons form a bridge-like structure between the two surface attached primers that flank the template sequence that has been copied. Exemplary reagents and conditions that can be used for bridge amplification are described, for example, in U.S. Pat. Nos. 5,641,658; 7,115,400; and 8,895,249; and/or U.S. Patent Publication Nos. 2002/0055100 A1, 2004/0096853 A1, 2004/0002090 A1, 2007/0128624 A1 and 2008/0009420 A1, each of which is incorporated herein by reference. Solid-phase PCR amplification can also be carried out with one of the amplification primers attached to a bead or other solid support and the second primer in solution. An exemplary format that uses a combination of a surface attached primer and soluble primer is the format used in emulsion PCR as described, for example, in Dressman et al., Proc. Natl. Acad. Sci. USA 100:8817-8822 (2003), WO 05/010145, or U.S. Patent Publication Nos. 2005/0130173 A1 or 2005/0064460 A1, each of which is incorporated herein by reference. Emulsion PCR is illustrative of the format and it will be understood that for purposes of the methods set forth herein the use of an emulsion is optional and indeed for several embodiments an emulsion is not used.

RCA techniques can be modified for use in a method of the present disclosure. Exemplary components that can be used in an RCA reaction and principles by which RCA produces amplicons are described, for example, in Lizardi et al., Nat. Genet. 19:225-232 (1998) and U.S. Patent Publication No. 2007/0099208 A1, each of which is incorporated herein by reference. Primers used for RCA can be in solution or attached to a bead or other solid support. The primers can be one or more of the universal primers described herein.

MDA techniques can be modified for use in a method of the present disclosure. Some basic principles and useful conditions for MDA are described, for example, in Dean et al., Proc Natl. Acad. Sci. USA 99:5261-66 (2002); Lage et al., Genome Research 13:294-307 (2003); Walker et al., Molecular Methods for Virus Detection, Academic Press, Inc., 1995; Walker et al., Nucl. Acids Res. 20:1691-96 (1992); U.S. Pat. Nos. 5,455,166; 5,130,238; and 6,214,587, each of which is incorporated herein by reference. Primers used for MDA can be in solution or attached to a bead or other solid support at an amplification site. Again, the primers can be one or more of the universal primers described herein.

In particular embodiments a combination of the above-exemplified amplification techniques can be used. For example, RCA and MDA can be used in a combination wherein RCA is used to generate a concatameric amplicon in solution (e.g. using solution-phase primers). The amplicon can then be used as a template for MDA using primers that are attached to a bead or other solid support (e.g. universal primers). In this example, amplicons produced after the combined RCA and MDA steps will be attached to the bead or other solid support.

Nucleic acid probes that are used in a method set forth herein or present in an apparatus or composition of the present disclosure can include barcode sequences, and for embodiments that include a plurality of different nucleic acid probes, each of the probes can include a different barcode sequence from other probes in the plurality. Barcode sequences can be any of a variety of lengths.

Longer sequences can generally accommodate a larger number and variety of barcodes for a population. Generally, all probes in a plurality will have the same length barcode (albeit with different sequences), but it is also possible to use different length barcodes for different probes. A barcode sequence can be at least 2, 4, 6, 8, 10, 12, 15, 20 or more nucleotides in length. Alternatively or additionally, the length of the barcode sequence can be at most 20, 15, 12, 10, 8, 6, 4 or fewer nucleotides. Examples of barcode sequences that can be used are set forth, for example in, U.S.

Patent Publication No. 2014/0342921 A1 and U.S. Pat. No. 8,460,865, each of which is incorporated herein by reference.

A method of the present disclosure can include a step of performing a nucleic acid detection reaction on a bead or other solid support to determine barcode sequences of nucleic acid probes that are located on the bead or other solid support. In many embodiments the probes are randomly located on the bead or other solid support and the nucleic acid detection reaction provides information to locate each of the different probes. Exemplary nucleic acid detection methods include, but are not limited to nucleic acid sequencing of a probe, hybridization of nucleic acids to a probe, ligation of nucleic acids that are hybridized to a probe, extension of nucleic acids that are hybridized to a probe, extension of a first nucleic acid that is hybridized to a probe followed by ligation of the extended nucleic acid to a second nucleic acid that is hybridized to the probe, or other methods known in the art such as those set forth in U.S. Pat. No. 8,288,103 or 8,486,625, each of which is incorporated herein by reference.

Sequencing techniques, such as sequencing-by-synthesis (SBS) techniques, are a useful method for determining barcode sequences. SBS can be carried out as follows. To initiate a first SBS cycle, one or more labeled nucleotides, DNA polymerase, SBS primers etc., can be contacted with one or more features on a bead or other solid support (e.g. feature(s) where nucleic acid probes are attached to the bead or other solid support). Those features where SBS primer extension causes a labeled nucleotide to be incorporated can be detected. Optionally, the nucleotides can include a reversible termination moiety that terminates further primer extension once a nucleotide has been added to the SBS primer. For example, a nucleotide analog having a reversible terminator moiety can be added to a primer such that subsequent extension cannot occur until a deblocking agent is delivered to remove the moiety. Thus, for embodiments that use reversible termination, a deblocking reagent can be delivered to the bead or other solid support (before or after detection occurs). Washes can be carried out between the various delivery steps. The cycle can then be repeated n times to extend the primer by n nucleotides, thereby detecting a sequence of length n. Exemplary SBS procedures, fluidic systems and detection platforms that can be readily adapted for use with a composition, apparatus or method of the present disclosure are described, for example, in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and U.S. Patent Publication No. 2008/0108082, each of which is incorporated herein by reference.

Other sequencing procedures that use cyclic reactions can be used, such as pyrosequencing. Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 1 1 (1), 3-1 1 (2001); Ronaghi et al. Science 281 (5375), 363 (1998); or U.S. Pat. Nos. 6,210,891, 6,258,568 or 6,274,320, each of which is incorporated herein by reference). In pyrosequencing, released PPi can be detected by being immediately converted to adenosine triphosphate (ATP) by ATP sulfurylase, and the level of ATP generated can be detected via luciferase-produced photons. Thus, the sequencing reaction can be monitored via a luminescence detection system.

Excitation radiation sources used for fluorescence based detection systems are not necessary for pyrosequencing procedures. Useful fluidic systems, detectors and procedures that can be used for application of pyrosequencing to apparatus, compositions or methods of the present disclosure are described, for example, in PCT Patent Publication No. WO2012/058096, US Patent Publication No. 2005/0191698 A1, or U.S. Pat. Nos. 7,595,883 or 7,244,559, each of which is incorporated herein by reference.

Sequencing-by-ligation reactions are also useful including, for example, those described in Shendure et al. Science 309:1728-1732 (2005); or U.S. Pat. Nos. 5,599,675 or 5,750,341, each of which is incorporated herein by reference. Some embodiments can include sequencing-by-hybridization procedures as described, for example, in Bains et al., Journal of Theoretical Biology 135(3), 303-7 (1988); Drmanac et al., Nature Biotechnology 16, 54-58 (1998); Fodor et al., Science 251 (4995), 767-773 (1995); or PCT Publication No. WO 1989/10977, each of which is incorporated herein by reference. In both sequencing-by-ligation and sequencing-by-hybridization procedures, target nucleic acids (or amplicons thereof) that are present at sites of an array are subjected to repeated cycles of oligonucleotide delivery and detection. Compositions, apparatus or methods set forth herein or in references cited herein can be readily adapted for sequencing-by-ligation or sequencing-by-hybridization procedures. Typically, the oligonucleotides are fluorescently labeled and can be detected using fluorescence detectors similar to those described with regard to SBS procedures herein or in references cited herein.

Some sequencing embodiments can utilize methods involving the real-time monitoring of DNA polymerase activity. For example, nucleotide incorporations can be detected through fluorescence resonance energy transfer (FRET) interactions between a fluorophore-bearing polymerase and 7-phosphate-labeled nucleotides, or with zeromode waveguides (ZMWs). Techniques and reagents for FRET-based sequencing are described, for example, in Levene et al. Science 299, 682-686 (2003); Lundquist et al. Opt. Lett. 33, 1026-1028 (2008); and Korlach et al. Proc. Natl. Acad. Sci. USA 105, 1 176-1 181 (2008), each of which is incorporated herein by reference.

Some sequencing embodiments include detection of a proton released upon incorporation of a nucleotide into an extension product. For example, sequencing based on detection of released protons can use an electrical detector and associated techniques that are commercially available from Ion Torrent (Guilford, Conn., a Life Technologies and Thermo Fisher subsidiary) or sequencing methods and systems described in U.S. Patent Publication Nos. 2009/0026082 A1; 2009/0127589 A1; 2010/0137143 A1; or U.S. Publication No. 2010/0282617 A1, each of which is incorporated herein by reference.

Nucleic acid hybridization techniques are also useful method for determining barcode sequences. In some cases combinatorial hybridization methods can be used such as those used for decoding of multiplex bead arrays (see, e.g., U.S. Pat. No. 8,460,865, which is incorporated herein by reference). Such methods utilize labelled nucleic acid decoder probes that are complementary to at least a portion of a barcode sequence. A hybridization reaction can be carried out using decoder probes having known labels such that the location where the labels end up on the bead or other solid support identifies the nucleic acid probes according to rules of nucleic acid complementarity. In some cases, pools of many different probes with distinguishable labels are used, thereby allowing a multiplex decoding operation. The number of different barcodes determined in a decoding operation can exceed the number of labels used for the decoding operation. For example, decoding can be carried out in several stages where each stage constitutes hybridization with a different pool of decoder probes. The same decoder probes can be present in different pools but the label that is present on each decoder probe can differ from pool to pool (i.e. each decoder probe is in a different “state” when in different pools).

Various combinations of these states and stages can be used to expand the number of barcodes that can be decoded well beyond the number of distinct labels available for decoding. Such combinatorial methods are set forth in further detail in U.S. Pat. No. 8,460,865 or Gunderson et al., Genome Research 14:870-877 (2004), each of which is incorporated herein by reference.

A method of the present disclosure can include a step of contacting a biological specimen (i.e., a sectioned tissue sample, optionally a cryosection) with a bead or other solid support that has nucleic acid probes attached thereto. In some embodiments, the nucleic acid probes are randomly located on the bead or other solid support. The identity and location of the nucleic acid probes may have been decoded prior to contacting the biological specimen with the bead or other solid support.

Alternatively, the identity and location of the nucleic acid probes can be determined after contacting the bead or other solid support with the biological specimen.

Bead-Attached Oligonucleotides

Certain aspects of the instant disclosure employ a nucleotide- or oligonucleotide-adorned bead, where the bead-attached oligonucleotide includes one or more of the following: a linker; an identical sequence for use as a sequencing priming site; a uniform or near-uniform nucleotide or oligonucleotide sequence; a Unique Molecular Identifier which differs for each priming site; an oligonucleotide redundant sequence for capturing polyadenylated mRNAs and priming reverse transcription (i.e., a poly-T sequence); and at least one oligonucleotide barcode which provides an substrate for spatial identification of an individual bead's position within a bead array. Exemplified bead-attached oligonucleotides of the instant disclosure include an oligonucleotide spatial barcode designed to be unique to each bead within a bead array (or at least wherein the majority of such barcodes are unique to a bead within a bead array—e.g., it is expressly contemplated here and elsewhere herein that a bead array possessing only a small fraction of beads (e.g., even up to 10%, 20%, 30% or 40% or more of total beads) having non-unique spatial barcodes (e.g., attributable to a relative lack of degeneracy within the bead population, e.g., due to a probabilistically determinable lack of sequence degeneracy calculated as possible within the bead population, as then compared to the number of sites across which the bead population is ultimately distributed and/or due to an artifact such as non-randomness of bead association occurring during pool-and-split rounds of oligonucleotide synthesis, etc.) could still yield high resolution transcriptome expression images, even while removing (or otherwise adjusting for) any beads that turn out to be redundant in barcode within the array). This spatial barcode provides a substrate for identification. Exemplified bead-attached oligonucleotides of the instant disclosure also include a linker (optionally a cleavable linker); a poly-dT sequence (herein, as a 3′ tail); a Unique Molecular Identifier (UMI) which differs for each priming site (as described below and as known in the art, e.g., see WO 2016/040476); a spatial barcode as described above and elsewhere herein; and a common sequence (“PCR handle”) to enable PCR amplification after “single-cell transcriptomes attached to microparticles” (STAMP) formation. As set forth in WO 2016/040476, mRNAs bind to poly-dT-presenting primers on their companion microparticle. At steps where mRNA sequence is to be identified, the mRNAs are reverse-transcribed into cDNAs, generating a set of beads called STAMPs. The barcoded STAMPs can then be amplified in pools for high-throughput mRNA-seq to analyze any desired number of beads (where each bead roughly corresponds to an approximately bead-sized area of cellular transcriptomes derived from the sectioned tissue sample (in the instant disclosure, 10 μm beads were used to produce resolutions approximating single cell feature sizes, as exemplified herein).

It is expressly contemplated that, instead of or in addition to the above-referenced poly-dT-presenting primers, oligonucleotide sequences designed for capture of a broader range of macromolecules as described here and elsewhere herein, can be used. In particular, oligonucleotide-directed capture of other types of macromolecules is also contemplated for the bead-attached oligonucleotides of the instant disclosure; for instance, a gene-specific capture sequence can be incorporated into oligonucleotide sequences (e.g., for purpose of capturing a full range of cell/tissue-associated RNAs including non-poly-A-tailed RNAs, such as tRNAs, miRNAs, etc., or for purpose of specifically capturing DNAs) and/or a loaded transposase can be used to capture, for example, DNA, and/or a specific sequence can be included to allow for specific capture of a DNA-barcoded antibody signal (not only allowing for assessment of protein distribution across a test sample using the compositions and methods of the instant disclosure, but also thereby, e.g., allowing for linkage of the spatial distributions of proteins to RNA expression).

Exemplary split-and-pool synthesis of the bead barcode: To generate the cell barcode, the pool of microparticles (here, microbeads) is repeatedly split into four equally sized oligonucleotide synthesis reactions, to which one of the four DNA bases is added, and then pooled together after each cycle, in a total of 12 split-pool cycles. The barcode synthesized on any individual bead reflects that bead's unique (or sufficiently unique) path through the series of synthesis reactions. The result is a pool of microparticles, each possessing one of 4¹² (16,777,216) possible sequences on its entire complement of primers. Extension of the split-pool process can provide for, e.g., production of an even greater number of possible spatial barcode sequences for use in the compositions and methods of the instant disclosure. However, as noted above, functional use of spatial barcodes does not require complete non-redundancy of spatial barcodes among all beads of a bead array. Rather, provided that the majority of such barcodes are unique to a bead within a bead array, it is expressly contemplated that a bead array possessing only a small fraction of beads (e.g., even up to 10%, 20%, 30% or 40% or more of total beads) having non-unique spatial barcodes (e.g., attributable to an artifact such as non-randomness of bead association having occurred during pool-and-split rounds of oligonucleotide synthesis, or simply to the likelihood that an array of a million beads derived from a ten million-fold complex library would still be expected to include a number of beads having redundant spatial barcodes in pairwise comparisons) could still yield high resolution transcriptome expression images, where removal or other adjustment (averaging or other such adjustment) of any beads that turn out to be redundant in barcode within the array could be simply performed, e.g., during in silico spatial location assignment and/or image generation.

Exemplary synthesis of a unique molecular identifier (UMI). Following the completion of the “split-and-pool” synthesis cycles described above for generation of spatial barcodes, all microparticles are together subjected to eight rounds of degenerate synthesis with all four DNA bases available during each cycle, such that each individual primer receives one of 48 (65,536) possible sequences (UMIs). A UMI is thereby provided that allows distinguishing between, e.g., individual bead-attached oligonucleotides upon the same bead which otherwise share a common spatial barcode (being that such oligonucleotides are attached to the same bead and therefore receive the same spatial barcode).

In some embodiments of the instant disclosure, the linker of a bead-attached oligonucleotide is a chemically-cleavable, straight-chain polymer. Optionally, the linker is a photolabile optionally substituted hydrocarbon polymer. In certain embodiments, the linker of a bead-attached oligonucleotide is a non-cleavable, straight-chain polymer. Optionally, the linker is a non-cleavable, optionally substituted hydrocarbon polymer. In certain embodiments, the linker is a polyethylene glycol. In one embodiment, the linker is a PEG-C3 to PEG-24.

A nucleic acid probe used in a composition or method set forth herein can include a target capture moiety. In particular embodiments, the target capture moiety is a target capture sequence. The target capture sequence is generally complementary to a target sequence such that target capture occurs by formation of a probe-target hybrid complex. A target capture sequence can be any of a variety of lengths including, for example, lengths exemplified above in the context of barcode sequences.

In certain embodiments, a plurality of different nucleic acid probes can include different target capture sequences that hybridize to different target nucleic acid sequences from a biological specimen. Different target capture sequences can be used to selectively bind to one or more desired target nucleic acids from a biological specimen. In some cases, the different nucleic acid probes can include a target capture sequence that is common to all or a subset of the probes on a solid support. For example, the nucleic acid probes on a solid support can have a poly A or poly T sequence. Such probes or amplicons thereof can hybridize to mRNA molecules, cDNA molecules or amplicons thereof that have poly A or poly T tails. Although the mRNA or cDNA species will have different target sequences, capture will be mediated by the common poly A or poly T sequence regions.

Any of a variety of target nucleic acids can be captured and analyzed in a method set forth herein including, but not limited to, messenger RNA (mRNA), copy DNA (cDNA), genomic DNA (gDNA), ribosomal RNA (rRNA) or transfer RNA (tRNA). Particular target sequences can be selected from databases and appropriate capture sequences designed using techniques and databases known in the art.

A method set forth herein can include a step of hybridizing nucleic acid probes, that are on a supported bead array, to target nucleic acids that are from portions of the biological specimen that are proximal to the probes. Generally, a target nucleic acid will flow or diffuse from a region of the biological specimen to an area of the probe-presenting bead array that is in proximity with that region of the specimen. Here the target nucleic acid will interact with nucleic acid probes that are proximal to the region of the specimen from which the target nucleic acid was released. A target-probe hybrid complex can form where the target nucleic acid encounters a complementary target capture sequence on a nucleic acid probe. The location of the target-probe hybrid complex will generally correlate with the region of the biological specimen from where the target nucleic acid was derived. In certain embodiments, the beads will include a plurality of nucleic acid probes, the biological specimen will release a plurality of target nucleic acids and a plurality of target-probe hybrids will be formed on the beads. The sequences of the target nucleic acids and their locations on the bead array will provide spatial information about the nucleic acid content of the biological specimen. Although the example above is described in the context of target nucleic acids that are released from a biological specimen, it will be understood that the target nucleic acids need not be released. Rather, the target nucleic acids may remain in contact with the biological specimen, for example, when they are attached to an exposed surface of the biological specimen in a way that the target nucleic acids can also bind to appropriate nucleic acid probes on the beads.

A method of the present disclosure can include a step of extending bead-attached probes to which target nucleic acids are hybridized. In embodiments where the probes include barcode sequences, the resulting extended probes will include the barcode sequences and sequences from the target nucleic acids (albeit in complementary form). The extended probes are thus spatially tagged versions of the target nucleic acids from the biological specimen. The sequences of the extended probes identify what nucleic acids are in the biological specimen and where in the biological specimen the target nucleic acids are located. It will be understood that other sequence elements that are present in the nucleic acid probes can also be included in the extended probes (see, e.g., description as provided elsewhere herein). Such elements include, for example, primer binding sites, cleavage sites, other tag sequences (e.g. sample identification tags), capture sequences, recognition sites for nucleic acid binding proteins or nucleic acid enzymes, or the like.

Extension of probes can be carried out using methods exemplified herein or otherwise known in the art for amplification of nucleic acids or sequencing of nucleic acids. In particular embodiments one or more nucleotides can be added to the 3′ end of a nucleic acid, for example, via polymerase catalysis (e.g. DNA polymerase, RNA polymerase or reverse transcriptase). Chemical or enzymatic methods can be used to add one or more nucleotide to the 3′ or 5′ end of a nucleic acid. One or more oligonucleotides can be added to the 3′ or 5′ end of a nucleic acid, for example, via chemical or enzymatic (e.g. ligase catalysis) methods. A nucleic acid can be extended in a template directed manner, whereby the product of extension is complementary to a template nucleic acid that is hybridized to the nucleic acid that is extended. In some embodiments, a DNA primer is extended by a reverse transcriptase using an RNA template, thereby producing a cDNA. Thus, an extended probe made in a method set forth herein can be a reverse transcribed DNA molecule. Exemplary methods for extending nucleic acids are set forth in US Pat. App. Publ. No. US 2005/0037393 A1 or U.S. Pat. No. 8,288,103 or 8,486,625, each of which is incorporated herein by reference.

All or part of a target nucleic acid that is hybridized to a nucleic acid probe can be copied by extension. For example, an extended probe can include at least, 1, 2, 5, 10, 25, 50, 100, 200, 500, 1000 or more nucleotides that are copied from a target nucleic acid. The length of the extension product can be controlled, for example, using reversibly terminated nucleotides in the extension reaction and running a limited number of extension cycles. The cycles can be run as exemplified for SBS techniques and the use of labeled nucleotides is not necessary.

Accordingly, an extended probe produced in a method set forth herein can include no more than 1000, 500, 200, 100, 50, 25, 10, 5, 2 or 1 nucleotides that are copied from a target nucleic acid. Of course extended probes can be any length within or outside of the ranges set forth above.

It will be understood that probes used in a method, composition or apparatus set forth herein need not be nucleic acids. Other molecules can be used such as proteins, carbohydrates, small molecules, particles or the like. Probes can be a combination of a nucleic acid component (e.g. having a barcode, primer binding site, cleavage site and/or other sequence element set forth herein) and another moiety (e.g. a moiety that captures or modifies a target nucleic acid).

A method set forth herein can further include a step of acquiring an image of a biological specimen that is in contact with a bead array. The solid support can be in any of a variety of states set forth herein. For example, the bead array can include attached nucleic acid probes or clusters derived from attached nucleic acid probes.

A method of the present disclosure can further include a step of removing one or more extended probes from a bead. In particular embodiments, the probes will have included a cleavage site such that the product of extending the probes will also include the cleavage site. Alternatively, a cleavage site can be introduced into a probe during a modification step. For example a cleavage site can be introduced into an extended probe during the extension step.

Exemplary cleavage sites include, but are not limited to, moieties that are susceptible to a chemical, enzymatic or physical process that results in bond breakage. For example, the location can be a nucleotide sequence that is recognized by an endonuclease. Suitable endonucleases and their recognition sequences are well known in the art and in many cases are even commercially available (e.g. from New England Biolabs, Beverley M A; ThermoFisher, Waltham, Mass. or Sigma Aldrich, St. Louis Mo.). A particularly useful endonuclease will break a bond in a nucleic acid strand at a site that is 3′-remote to its binding site in the nucleic acid, examples of which include Type II or Type 1 is restriction endonucleases. In some embodiments an endonuclease will cut only one strand in a duplex nucleic acid (e.g. a nicking enzyme). Examples of endonucleases that cleave only one strand include Nt.BstNBI and Nt.Alwl.

In some embodiments, a cleavage site is an abasic site or a nucleotide that has a base that is susceptible to being removed to create an abasic site. Examples of nucleotides that are susceptible to being removed to form an abasic site include uracil and 8-oxo-guanine. Abasic sites can be created by hydrolysis of nucleotide residues using chemical or enzymatic reagents. Once formed, abasic sites may be cleaved (e.g. by treatment with an endonuclease or other single-stranded cleaving enzyme, exposure to heat or alkali), providing a means for site-specific cleavage of a nucleic acid. An abasic site may be created at a uracil nucleotide on one strand of a nucleic acid. The enzyme uracil DNA glycosylase (UDG) may be used to remove the uracil base, generating an abasic site on the strand. The nucleic acid strand that has the abasic site may then be cleaved at the abasic site by treatment with endonuclease (e.g. EndolV endonuclease, AP lyase, FPG glycosylase/AP lyase, EndoVIII glycosylase/AP lyase), heat or alkali. In a particular embodiment, the USER™ reagent available from New England Biolabs is used for the creation of a single nucleotide gap at a uracil base in a nucleic acid.

Abasic sites may also be generated at non-natural/modified deoxyribonucleotides other than uracil and cleaved in an analogous manner by treatment with endonuclease, heat or alkali. For example, 8-oxo-guanine can be converted to an abasic site by exposure to FPG glycosylase. Deoxyinosine can be converted to an abasic site by exposure to AlkA glycosylase. The abasic sites thus generated may then be cleaved, typically by treatment with a suitable endonuclease (e.g. EndolV or AP lyase).

Other examples of cleavage sites and methods that can be used to cleave nucleic acids are set forth, for example, in U.S. Pat. No. 7,960,120, which is incorporated herein by reference.

Modified nucleic acid probes (e.g. extended nucleic acid probes) that are released from a solid support can be pooled to form a fluidic mixture. The mixture can include, for example, at least 10, 100, 1×10³, 1×10⁴, 1×10⁵, 1×10⁶, 1×10⁷, 1×10⁸, 1×10⁹ or more different modified probes.

Alternatively or additionally, a fluidic mixture can include at most 1×10⁹, 1×10⁸, 1×10⁷, 1×10⁶, 1×10⁵, 1×10⁴, 1×10³, 100, 10 or fewer different modified probes. The fluidic mixture can be manipulated to allow detection of the modified nucleic acid probes. For example, the modified nucleic acid probes can be separated spatially on a second solid support (i.e. different from the bead array and/or adhered solid support from which the nucleic acid probes were released after having been contacted with a biological specimen and modified), or the probes can be separated temporally in a fluid stream.

Modified nucleic acid probes (e.g. extended nucleic acid probes) can be separated on a bead or other solid support in a capture or detection method commonly employed for microarray-based techniques or nucleic acid sequencing techniques such as those set forth previously and/or otherwise described herein. For example, modified probes can be attached to a microarray by hybridization to complementary nucleic acids. The modified probes can be attached to beads or to a flow cell surface and optionally amplified as is carried out in many nucleic acid sequencing platforms. Modified probes can be separated in a fluid stream using a microfluidic device, droplet manipulation device, or flow cytometer. Typically, detection is carried out on these separation devices, but detection is not necessary in all embodiments.

The number of bead-attached oligonucleotides present upon an individual bead can vary across a wide range, e.g., from tens to thousands, or millions, or more. Due to the transcriptome profiling nature of the instant disclosure, it is generally preferred to pack as many capture oligonucleotides as spatially and sterically (as well as economically) possible onto an individual bead (i.e., thousands, tens of thousands, or more, of oligonucleotides per individual bead), provided that mRNA capture from a contacted tissue is optimized. It is contemplated that optimization of the oligonucleotide-per-bead metric can be readily performed by one of ordinary skill in the art.

It is further expressly contemplated that in addition to the above-described sequence features, oligonucleotides of the instant disclosure can possess any number of other art-recognized features while remaining within the scope of the instant disclosure.

Capture Material

In certain aspects of the instant disclosure, a capture material is employed to associate a bead array with a solid support (e.g., a glass slide). In some embodiments, the capture material is a liquid electrical tape. An exemplary liquid electrical tape of the instant disclosure is Permatex™ liquid electrical tape, which is a weatherproof protectant for wiring and electrical connections. Liquid capture material such as liquid tape can be applied as a liquid, which then dries to a vinyl polymer that resists dirt, dust, chemicals, and moisture, ensuring that applied beads are attached to a capture material-coated slide in a dry condition. Without wishing to be bound by theory, it is believed that one advantage of the instant methods is that the oligonucleotide-coated beads used in certain embodiments of the invention, which are attached to a solid support (e.g., a slide surface via use, e.g., of electrical tape as a capture material) are maintained in a dry state that optimizes transfer of RNA (or other macromolecule) from a section of a tissue to a bead-coated surface (again without wishing to be bound by theory, such transfer is currently believed to occur via capillary action at the scale of the microbead-section interface surface). It is believed that this highly efficient and direct transfer of cellular RNAs (i.e., the transcriptome of cells found within sectioned tissues) or other macromolecules to microbeads (where each microbead respectively possesses thousands of oligonucleotides capable of capturing oligoribonucleotides, e.g., transcripts) arrayed upon a solid support—where the transfer occurs upon an otherwise dry surface, therefore limiting and/or eliminating diffusive properties—is what imparts the instant methods and compositions with extremely high resolution (i.e., resolution at 10-50 μm spacing across a two-dimensional image of a section) of assessment of the cellular transcriptomes (or other macromolecules) of assayed tissue sections.

It is contemplated that beads of the instant disclosure can be applied to a capture material-coated solid support, either immediately upon deposit of capture material to the solid support, or following an initial drying period for the capture material. Capture materials of the instant disclosure can be applied by any of a number of methods, including brushed onto the solid support, sprayed onto the solid support, or the like, or via submersion of the solid support in the capture material. For certain forms of liquid capture material, use of a brush top applicator can allow coverage without gaps and can enable access to tight spaces, which offers advantages in certain embodiments over forms of capture material (i.e., tape) that are applied in a non-liquid state.

While liquid electrical tape has been exemplified as a capture material for use in the methods and compositions of the instant disclosure, other capture materials are also contemplated for such use, including any art-recognized glue or other reagent that is (a) spreadable and/or depositable upon a solid surface (e.g., upon a slide, optionally a slide that allows for light transmission through the slide, e.g., a microscope slide) and (b) capable of binding or otherwise capturing a population of beads of 1-100 μm size. Exemplary other capture materials that are expressly contemplated include latex such as cis-1,4-polyisoprene and other rubbers, as well as elastomers (which are generally defined as polymers that possess viscoelasticity (i.e., both viscosity and elasticity), very weak inter-molecular forces, and generally low Young's modulus and high failure strain compared with other materials), including artificial elastomers (e.g., neoprene) and/or silicone elastomers. Acrylate polymers (e.g., scotch tape) are also expressly contemplated, e.g., for use as a capture material of the instant disclosure.

In Situ Sequencing

In certain aspects of the disclosure, in situ sequencing is performed upon a bead array affixed to a surface, which can be performed by any art-recognized mode of parallel (optionally massively parallel) in situ sequencing, examples of which particularly include the previously described SOLiD™ method, which is a sequencing-by-ligation technique that can be performed in situ upon a solid support (refer, e.g., to Voelkerding et al, Clinical Chem., 55-641-658, 2009; U.S. Pat. Nos. 5,912,148; and 6,130,073, which are incorporated herein by reference in their entireties). In certain embodiments of the instant disclosure, such sequencing can be performed upon a bead array present on a standard microscope slide, optionally using a standard microscope fitted with sufficient computing power to track and associate individual sequences during progressive rounds of detection, with their spatial position(s). The instant disclosure also employed custom fluidics, incubation times, enzymatic mixes and imaging setup in performing in situ sequencing.

Tissue Samples and Sectioning

In some embodiments, a tissue section is employed. The tissue can be derived from a multicellular organism. Exemplary multicellular organisms include, but are not limited to a mammal, plant, algae, nematode, insect, fish, reptile, amphibian, fungi or Plasmodium falciparum. Exemplary species are set forth previously herein or known in the art. The tissue can be freshly excised from an organism or it may have been previously preserved for example by freezing, embedding in a material such as paraffin (e.g. formalin fixed paraffin embedded samples), formalin fixation, infiltration, dehydration or the like. Optionally, a tissue section can be sectioned, optionally cryosectioned, using techniques and compositions as described herein and as known in the art. As a further option, a tissue can be permeabilized and the cells of the tissue lysed. Any of a variety of art-recognized lysis treatments can be used. Target nucleic acids that are released from a tissue that is permeabilized can be captured by nucleic acid probes, as described herein and as known in the art.

A tissue can be prepared in any convenient or desired way for its use in a method, composition or apparatus herein. Fresh, frozen, fixed or unfixed tissues can be used. A tissue can be fixed or embedded using methods described herein or known in the art.

A tissue sample for use herein, can be fixed by deep freezing at temperature suitable to maintain or preserve the integrity of the tissue structure, e.g. less than −20° C. In another example, a tissue can be prepared using formalin-fixation and paraffin embedding (FFPE) methods which are known in the art. Other fixatives and/or embedding materials can be used as desired. A fixed or embedded tissue sample can be sectioned, i.e. thinly sliced, using known methods. For example, a tissue sample can be sectioned using a chilled microtome or cryostat, set at a temperature suitable to maintain both the structural integrity of the tissue sample and the chemical properties of the nucleic acids in the sample. Exemplary additional fixatives that are expressly contemplated include alcohol fixation (e.g., methanol fixation, ethanol fixation), glutaraldehyde fixation and paraformaldehyde fixation.

In some embodiments, a tissue sample will be treated to remove embedding material (e.g. to remove paraffin or formalin) from the sample prior to release, capture or modification of nucleic acids. This can be achieved by contacting the sample with an appropriate solvent (e.g. xylene and ethanol washes). Treatment can occur prior to contacting the tissue sample with a solid support-captured bead array as set forth herein or the treatment can occur while the tissue sample is on the solid support-captured bead array.

Exemplary methods for manipulating tissues for use with solid supports to which nucleic acids are attached are set forth in US Pat. App. Publ. No. 2014/0066318 A1, which is incorporated herein by reference.

The thickness of a tissue sample or other biological specimen that is contacted with a bead array in a method, composition or apparatus set forth herein can be any suitable thickness desired. In representative embodiments, the thickness will be at least 0.1 μm, 0.25 μm, 0.5 μm, 0.75 μm, 1 μm, 5 μm, 10 μm, 50 μm, 100 μm or thicker. Alternatively or additionally, the thickness of a tissue sample that is contacted with bead array will be no more than 100 μm, 50 μm, 10 μm, 5 μm, 1 μm, 0.5 μm, 0.25 μm, 0.1 μm or thinner.

A particularly relevant source for a tissue sample is a human being. The sample can be derived from an organ, including for example, an organ of the central nervous system such as brain, brainstem, cerebellum, spinal cord, cranial nerve, or spinal nerve; an organ of the musculoskeletal system such as muscle, bone, tendon or ligament; an organ of the digestive system such as salivary gland, pharynx, esophagus, stomach, small intestine, large intestine, liver, gallbladder or pancreas; an organ of the respiratory system such as larynx, trachea, bronchi, lungs or diaphragm; an organ of the urinary system such as kidney, ureter, bladder or urethra; a reproductive organ such as ovary, fallopian tube, uterus, vagina, placenta, testicle, epididymis, vas deferens, seminal vesicle, prostate, penis or scrotum; an organ of the endocrine system such as pituitary gland, pineal gland, thyroid gland, parathyroid gland, or adrenal gland; an organ of the circulatory system such as heart, artery, vein or capillary; an organ of the lymphatic system such as lymphatic vessel, lymph node, bone marrow, thymus or spleen; a sensory organ such as eye, ear, nose, or tongue; or an organ of the integument such as skin, subcutaneous tissue or mammary gland. In some embodiments, a tissue sample is obtained from a bodily fluid or excreta such as blood, lymph, tears, sweat, saliva, semen, vaginal secretion, ear wax, fecal matter or urine.

A sample from a human can be considered (or suspected) healthy or diseased when used. In some cases, two samples can be used: a first being considered diseased and a second being considered as healthy (e.g. for use as a healthy control). Any of a variety of conditions can be evaluated, including but not limited to, an autoimmune disease, cancer, cystic fibrosis, aneuploidy, pathogenic infection, psychological condition, hepatitis, diabetes, sexually transmitted disease, heart disease, stroke, cardiovascular disease, multiple sclerosis or muscular dystrophy. Certain contemplated conditions include genetic conditions or conditions associated with pathogens having identifiable genetic signatures.

Macromolecules

In addition to the poly-A-tailed RNAs captured by poly-dT sequences in certain exemplified embodiments of the instant disclosure, it is expressly contemplated that the instant compositions and methods can be applied to obtain spatially-resolvable abundance data (in concert with extended length TCR sequences) for a wide range of macromolecules, including not only poly-A-tailed RNAs/transcripts, but also, e.g., non-poly-A-tailed RNAs (e.g., tRNAs, miRNAs, etc.; optionally specifically captured using sequence-specific oligonucleotide sequences), DNAs (including, e.g., capture via gene-specific oligonucleotides, loaded transposases, etc.), and proteins (including, e.g., DNA-barcoded antibodies, optionally where a DNA barcode effectively tags a capture antibody for detection, allowing for direct comparison of spatial distribution(s) of antibodies and/or antibody-captured proteins with spatially-resolvable expression profiling that also can be performed upon the test sample via use of the compositions and methods of the instant disclosure. Accordingly, the range of macromolecules expressly contemplated for capture using the compositions and methods of the instant disclosure includes all forms of RNA (including, e.g., transcripts, tRNAs, rRNAs, miRNAs, etc.), DNAs (including, e.g., genomic DNAs, barcode DNAs, etc.) and proteins (including, e.g., antibodies that are tagged for binding and detection and/or other forms of protein, optionally including proteins captured by antibodies). In one embodiment, proteins can be profiled using a library of DNA-barcoded antibodies to stain a tissue, before capturing proteins on the spatial array (refer to Cellular Indexing of Transcriptome and Epitopes by sequencing (CITE-seq), which combines unbiased genome-wide expression profiling with the measurement of specific protein markers in thousands of single cells using droplet microfluidics. In brief, monoclonal antibodies are conjugated to oligonucleotides containing unique antibody identifier sequences; a cell suspension is then labeled with the oligo-tagged antibodies and single cells are subsequently encapsulated into nanoliter-sized aqueous droplets in a microfluidic apparatus. In each droplet, antibody and cDNA molecules are indexed with the same unique (or sufficiently unique) barcode and are converted into libraries that are amplified independently and mixed in appropriate proportions for sequencing in the same lane. Stoeckius and Smibert. Protocol Exchange (2017) doi: 10.1038/protex.2017.068). Additionally, proteins may be adsorbed onto the beads nonspecifically, or through chemical capture (such as amine reactive chemistry or crosslinkers), the beads may be sorted into wells and the proteins quantitated by standard measures (antibodies, ELISA, etc), and then followed by sequencing of the paired bead sequences and the spatial locations reconstructed.

Application of Wash Solution to Bead Array (Optional)

In certain embodiments, a solid support-captured bead array is washed after exposure of the bead array to a sectioned tissue (optionally, the sectioned tissue is removed prior to or during application of a wash solution). For example, a solid support-captured bead array of the instant disclosure can be submerged in a buffered salt solution (or other stabilizing solution) after contacting the bead array with a sectioned tissue sample. Exemplified buffered salt solutions include saline-sodium citrate (SSC), for example at a NaCl concentration of about 0.2 M to 5 M NaCl, optionally at about 0.5 to 3 M NaCl, optionally at about 1 M NaCl. Without wishing to be bound by theory, as exemplified, exposure of a transcriptome-bound bead array to a saline solution (or other stabilizing solution) is believed to stabilize bead-attached capture probe-sample RNA (i.e., transcript) interactions, likely by blocking RNA degradation and/or other degradative processes. While SSC has been exemplified in the processes of the instant disclosure, use of other types of buffered solutions is expressly contemplated, including, e.g. PBS, Tris buffered saline and/or Tris buffer, as well as, more broadly, any aqueous buffer possessing a pH between 4 and 10 and salt between 0-1 osmolarity.

Wash solutions can contain various additives, such as surfactants (e.g. detergents), enzymes (e.g. proteases and collagenases), cleavage reagents, or the like, to facilitate removal of the specimen. In some embodiments, the solid support is treated with a solution comprising a proteinase enzyme. Alternatively or additionally, the solution can include cellulase, hemicelluase or chitinase enzymes (e.g. if desiring to remove a tissue sample from a plant or fungal source). In some cases, the temperature of a wash solution will be at least 30° C., 35° C., 50° C., 60° C. or 90° C. Conditions can be selected for removal of a biological specimen while not denaturing hybrid complexes formed between target nucleic acids and solid support-attached nucleic acid probes.

Sequencing Methods

Some of the methods and compositions provided herein employ methods of sequencing nucleic acids. A number of DNA sequencing techniques are known in the art, including fluorescence-based sequencing methodologies (See, e.g., Birren et al, Genome Analysis Analyzing DNA, 1, Cold Spring Harbor, N.Y., which is incorporated herein by reference in its entirety). In some embodiments, automated sequencing techniques understood in that art are utilized. In some embodiments, parallel sequencing of partitioned amplicons can be utilized (PCT Publication No WO2006084132, which is incorporated herein by reference in its entirety). In some embodiments, DNA sequencing is achieved by parallel oligonucleotide extension (See, e.g., U.S. Pat. Nos. 5,750,341; 6,306,597, which are incorporated herein by reference in their entireties). Additional examples of sequencing techniques include the Church polony technology (Mitra et al, 2003, Analytical Biochemistry 320, 55-65; Shendure et al, 2005 Science 309, 1728-1732; U.S. Pat. Nos. 6,432,360, 6,485,944, 6,511,803, which are incorporated by reference), the 454 picotiter pyrosequencing technology (Margulies et al, 2005 Nature 437, 376-380; US 20050130173, which are incorporated herein by reference in their entireties), the Solexa single base addition technology (Bennett et al, 2005, Pharmacogenomics, 6, 373-382; U.S. Pat. Nos. 6,787,308; 6,833,246, which are incorporated herein by reference in their entireties), the Lynx massively parallel signature sequencing technology (Brenner et al. (2000). Nat. Biotechnol. 18:630-634; U.S. Pat. Nos. 5,695,934; 5,714,330, which are incorporated herein by reference in their entireties), and the Adessi PCR colony technology (Adessi et al. (2000). Nucleic Acid Res. 28, E87; WO 00018957, which are incorporated herein by reference in their entireties).

Next-generation sequencing (NGS) methods can be employed in certain aspects of the instant disclosure to obtain a high volume of sequence information (such as are particularly required to perform deep sequencing of bead-associated RNAs following capture of RNAs from sections) in a highly efficient and cost effective manner. NGS methods share the common feature of massively parallel, high-throughput strategies, with the goal of lower costs in comparison to older sequencing methods (see, e.g., Voelkerding et al, Clinical Chem., 55: 641-658, 2009; MacLean et al, Nature Rev. Microbiol, 7-287-296; which are incorporated herein by reference in their entireties). NGS methods can be broadly divided into those that typically use template amplification and those that do not. Amplification-utilizing methods include pyrosequencing commercialized by Roche as the 454 technology platforms (e.g., GS 20 and GS FLX), the Solexa platform commercialized by Illumina, and the Supported Oligonucleotide Ligation and Detection (SOLiD™) platform commercialized by Applied Biosystems. Non-amplification approaches, also known as single-molecule sequencing, are exemplified by the HeliScope platform commercialized by Helicos Biosciences, SMRT sequencing commercialized by Pacific Biosciences, and emerging platforms marketed by VisiGen and Oxford Nanopore Technologies Ltd.

In pyrosequencing (U.S. Pat. Nos. 6,210,891; 6,258,568, which are incorporated herein by reference in their entireties), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al, Clinical Chem., 55-641-658, 2009; MacLean et al, Nature Rev. Microbiol, 7:287-296; U.S. Pat. Nos. 6,833,246; 7,115,400; 6,969,488, which are incorporated herein by reference in their entireties), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluorophore and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al, Clinical Chem., 55: 641-658, 2009; U.S. Pat. Nos. 5,912,148; and 6,130,073, which are incorporated herein by reference in their entireties) can initially involve fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, nanopore sequencing is employed (see, e.g., Astier et al, J. Am. Chem. Soc. 2006 Feb. 8; 128(5): 1705-10, which is incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore (or as individual nucleotides pass through the nanopore in the case of exonuclease-based techniques), this causes a change in the magnitude of the current through the nanopore that is distinct for each of the four bases, thereby allowing the sequence of the DNA molecule to be determined.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, which are incorporated herein by reference in their entireties). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per base accuracy of the Ion Torrent sequencer is approximately 99.6% for 50 base reads, with approximately 100 Mb generated per run. The read-length is 100 base pairs. The accuracy for homopolymer repeats of 5 repeats in length is approximately 98%. The benefits of ion semiconductor sequencing are rapid sequencing speed and low upfront and operating costs.

Imaging/Image Assembly

With spatial barcodes of individual beads identified, and with sequences of those RNAs captured by individual bead-attached oligonucleotides (capture probes) also identified, high-resolution images that localize sites of RNA expression can be readily constructed in silico. In certain embodiments, the spatial locations of a large number of beads within an array can first be assigned to an image location, with all associated RNA sequence (expression) data also assigned to that position (optionally, effectively de-coupling the spatial barcode from the array/matrix of RNA sequence information associated with a given site/bead, once the spatial barcode has been used to assign the RNA sequence information to an array position). High resolution images representing the extent of capture of individual or grouped RNAs/transcripts across the various spatial positions of the arrays can then be generated using the underlying RNA sequence information (which was at least originally bead-associated). Images (i.e., pixel coloring and/or intensities) can be adjusted and/or normalized using any (or any number of) art-recognized technique(s) deemed appropriate by one of ordinary skill in the art.

In certain embodiments, a high-resolution image of the instant disclosure is an image in which discrete features (e.g., pixels) of the image are spaced at 50 μm or less. In some embodiments, the spacing of discrete features within the image is at 40 μm or less, optionally 30 μm or less, optionally 20 μm or less, optionally 15 μm or less, optionally 10 μm or less, optionally 9 μm or less, optionally 8 μm or less, optionally 7 μm or less, optionally 6 μm or less, optionally 5 μm or less, optionally 4 μm or less, optionally 3 μm or less, optionally 2 μm or less, or optionally 1 μm or less.

Images can be obtained using detection devices known in the art. Examples include microscopes configured for light, bright field, dark field, phase contrast, fluorescence, reflection, interference, or confocal imaging. A biological specimen can be stained prior to imaging to provide contrast between different regions or cells. In some embodiments, more than one stain can be used to image different aspects of the specimen (e.g. different regions of a tissue, different cells, specific subcellular components or the like). In other embodiments, a biological specimen can be imaged without staining.

In particular embodiments, a fluorescence microscope (e.g. a confocal fluorescent microscope) can be used to detect a biological specimen that is fluorescent, for example, by virtue of a fluorescent label. Fluorescent specimens can also be imaged using a nucleic acid sequencing device having optics for fluorescent detection such as a Genome Analyzer®, MiSeq®, NextSeq® or HiSeq® platform device commercialized by Illumina, Inc. (San Diego, Calif.); or a SOLiD™ sequencing platform commercialized by Life Technologies (Carlsbad, Calif.). Other imaging optics that can be used include those that are found in the detection devices described in Bentley et al., Nature 456:53-59 (2008), PCT Publ. Nos. WO 91/06678, WO 04/018497 or WO 07/123744; U.S. Pat. Nos. 7,057,026, 7,329,492, 7,211,414, 7,315,019 or 7,405,281, and US Pat. App. Publ. No. 2008/0108082, each of which is incorporated herein by reference.

An image of a biological specimen can be obtained at a desired resolution, for example, to distinguish tissues, cells or subcellular components. Accordingly, the resolution can be sufficient to distinguish components of a biological specimen that are separated by at least 0.5 μm, 1 μm, 5 μm, 10 μm, 50 μm, 100 μm, 500 μm, 1 mm or more. Alternatively or additionally, the resolution can be set to distinguish components of a biological specimen that are separated by at least 1 mm, 500 μm, 100 μm, 50 μm, 10 μm, 5 μm, 1 μm, 0.5 μm or less.

A method set forth herein can include a step of correlating locations in an image of a biological specimen with barcode sequences of nucleic acid probes that are attached to individual beads to which the biological specimen is, was or will be contacted. Accordingly, characteristics of the biological specimen that are identifiable in the image can be correlated with the nucleic acids that are found to be present in their proximity. Any of a variety of morphological characteristics can be used in such a correlation, including for example, cell shape, cell size, tissue shape, staining patterns, presence of particular proteins (e.g. as detected by immunohistochemical stains) or other characteristics that are routinely evaluated in pathology or research applications. Accordingly, the biological state of a tissue or its components as determined by visual observation can be correlated with molecular biological characteristics as determined by spatially resolved nucleic acid analysis.

A solid support upon which a biological specimen is imaged can include fiducial markers to facilitate determination of the orientation of the specimen or the image thereof in relation to probes that are attached to the solid support. Exemplary fiducials include, but are not limited to beads (with or without fluorescent moieties or moieties such as nucleic acids to which labeled probes can be bound), fluorescent molecules attached at known or determinable features, or structures that combine morphological shapes with fluorescent moieties. Exemplary fiducials are set forth in US Pat. App. Publ. No. 2002/0150909 A1 or U.S. patent application Ser. No. 14/530,299, each of which is incorporated herein by reference. One or more fiducials are preferably visible while obtaining an image of a biological specimen. Preferably, the solid support includes at least 2, 3, 4, 5, 10, 25, 50, 100 or more fiducial markers. The fiducials can be provided in a pattern, for example, along an outer edge of a solid support or perimeter of a location where a biological specimen resides. In one embodiment, one or more fiducials are detected using the same imaging conditions used to visualize a biological specimen. However if desired separate images can be obtained (e.g. one image of the biological specimen and another image of the fiducials) and the images can be aligned to each other.

### Kits

The instant disclosure also provides kits containing agents of this disclosure for use in the methods of the present disclosure. Kits of the instant disclosure may include one or more containers comprising an agent (e.g., a capture material, such as liquid electrical tape) and/or composition (e.g., a slide-captured bead array) of this disclosure. In some embodiments, the kits further include instructions for use in accordance with the methods of this disclosure. In some embodiments, these instructions comprise a description of administration of the agent to diagnose, e.g., a disease and/or malignancy. In some embodiments, the instructions comprise a description of how to create a tissue section, form a spatially-defined (or simply spatially definable, pending performance of a step that defines the spatial resolution of the bead array) bead array, contact a tissue section with a spatially-defined bead array and/or obtain captured, tissue section-derived transcript sequence from the spatially-defined bead array. The kit may further comprise a description of selecting an individual suitable for treatment based on identifying whether that subject has a certain pattern of expression of one or more transcripts in a section sample.

The instructions generally include information as to dosage, dosing schedule, and route of administration for the intended use/treatment. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for staging a section and/or diagnosing a specific expression pattern in a section. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. The container may further comprise a pharmaceutically active agent.

Kits may optionally provide additional components such as buffers and interpretive information. Normally, the kit comprises a container and a label or package insert(s) on or associated with the container.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims. Standard techniques well known in the art or the techniques specifically described below were utilized.

### EXAMPLES

### Example 1: Materials and Methods

### Beads:

Beads were produced by the ChemGenes Corporation on one of two polystyrene supports (Agilent and Custom Polystyrene supports from AM Biotech). Beads were used with one of the two following sequences:

Sequence 1:
(SEQ ID NO: 1)
5′-PEG Linker-TTTT-PCT-GCCGGTAATACGACTC
ACTATAGGGCTACACGACGCTCTTCCGATCTJJJJJJTC
TTCAGCGTTCCCGAGAJJJJJJJNNNNNNNNT30
Sequence 2:
(SEQ ID NO: 2)
5′-Linker-TTTTTTTTGCCGGGGCTACACGACGCTCT
TCCGATCTJJJJJJJJTCTTCAGCGTTCCCGAGAJJJJJ
JJNNNNNNNNT30

Here, PCT represents a photocleavable thymidine; J bases represent bases generated by split-pool barcoding, such that every oligo on a given bead has the same J bases; Ns represent bases generated by mixing, so every oligo on a given bead has different N bases; and T30 represents a string of 30 thymidines. The two sequences corresponded to different bead batches, which were not found to differ significantly in terms of the number of transcripts per bead.

### Puck Preparation:

Pucks were prepared as follows. Glass coverslips (Bioptechs, 40-1313-0319) were attached to a miniature centrifuge (USA Scientific 2621-0016) using double sided tape. Subsequently, the coverslip was cleaned by spraying with 70% ethanol and wiping with lens paper (VWR 52846-007) A spray-on silicone formulation was then sprayed onto the coverslip, the cover to the minifuge was closed, and the minifuge was turned on for 10 seconds. The minifuge was then turned off and the cover opened, and liquid tape (Performix 24122000) was sprayed onto the coverslip. The minifuge was again closed and turned on for 10 seconds. The coverslip was then carefully removed from the minifuge, and a gasket (Grace Biolabs, CW-50R-1.0) was placed on top of the coverslip and pressed down. Beads were then diluted to a concentration of approximately 100,000 beads/μL in ultrapure water (Thermofisher, 10977015). Beads were pelleted and resuspended twice in ultrapure water, and 10 uL of the resulting solution was pipetted into each position on the gasket. The coverslip-gasket filled with beads was then put into a spinning bucket centrifuge, preheated to 40° C., and centrifuged at 850 g for at least 30 minutes until the surface was dry.

Subsequently, the coverslip was removed from the centrifuge and the gasket was carefully removed. Gentle pipetting of water directly onto the pelleted bead pucks removed all beads except for those directly in contact with the liquid tape layer. Beads removed in this way could be stored at 4° C. for later use. As much water was removed from the resulting pucks as possible, and the pucks were left to dry.

### Puck Sequencing:

Puck sequencing for exemplification of the original “Slide-seq” technique was performed using SOLiD™ chemistry in a Bioptechs FCS2 flowcell using a RP-1 peristaltic pump (Rainin), and a modular valve positioner (Hamilton). Flow rates between 1 mL/min and 3 mL/min were typical. Imaging was performed using a Nikon Eclipse Ti microscope with a Yokogawa CSU-W1 confocal scanner unit and an Andor Zyla 4.2 Plus camera. Images were acquired using a Nikon Plan Apo 10×/0.45 objective. After each ligation, four images were acquired: one using a 488 nm laser and a 525/36 emission filter (MVI, 77074803); one using a 561 nm laser and a 582/15 emission filter (MVI, FF01-582/15-25); one using a 561 nm laser and a 624/40 emission filter (MVI, FF01-624/40-25); and one using a 647 nm laser and a 705/72 emission filter (MVI, 77074329). The final stitched images were 6030 pixels by 6030 pixels.

Sequencing consisted of three steps: primer hybridization, ligation, and stripping. During primer hybridization, a primer was flowed into the flowcell at 5 μM concentration in 4×SSC, and was allowed to sit for 20 minutes. Subsequently, the flowcell was washed in 3 mL of SOLiD buffer F. Following instrument buffer wash, ligation mix was flowed into the chamber and allowed to sit for 20 minutes, before being flowed back into its original reservoir. Ligation mix was reused for ˜10 ligations, before being replenished. Following ligation, the flowcell was washed again in instrument buffer, and 1.5 mL of SOLiD buffer C was then flowed in, followed by 1.5 mL of SOLiD buffer B, and this step was repeated once again, to cleave the SOLiD sequencing oligo. The flowcell was then washed in instrument buffer and the ligation step was repeated. After the second ligation step, 10 mL of 80% formamide in water was flowed into the flowcell and left for 10 minutes. The flowcell was then washed in instrument buffer, and the process was repeated with the next primer.

### Ligation Mix:

### 1×T4 DNA Ligase Buffer (Enzymatics)

### 6 U/uL T4 DNA Ligase (Rapid) (Enzymatics)

40× dilution of SOLiD SR-75 sequencing oligo.

Application of long-read sequencing (LRS) approaches for purpose of obtaining individual sequence read lengths that span TCR variable regions while also providing spatial/bead tag identities, molecular identifiers and/or other identifying sequence information (e.g., sequence barcodes) is also contemplated and is described elsewhere herein.

### Image Processing and Basecalling:

All image processing was performed using a custom-built processing suite in Matlab. Briefly, one image was acquired for puck after each ligation, and each image contained four color channels. First, color channels were co-registered to each other by thresholding the images and maximizing the cross-correlation between the thresholded images. Subsequently, for each puck, the images of each ligation were registered to the image of the first ligation using a SIFT-RANSAC image registration algorithm based on the VLFeat SIFT package in Matlab. Registered images were then basecalled on a pixel-wise basis, as follows. First, the intensities in the Cy3 channel were multiplied by a factor of 0.5 and subtracted from the intensities in the TxR channel, which accounts for cross-talk between the channels which resulted from the excitation of TxR using the 561 nm laser. Furthermore, for even-numbered ligations, the image of the previous ligation was multiplied by a factor of 0.4 and then subtracted on a channel-by-channel basis from the image of the even ligation. Each pixel was then called by intensity. For pucks made using the 180402 bead batch, the expected base balance was further enforced by including an additional step in which the intensities of the dimmest channels were progressively increased until each channel accounted for between 20% and 30% of the pixels in the center of the image.

Beads were subsequently identified from the basecalled images as follows. Each pixel was assigned a number, the base 5 representation of which corresponds to the bases that were called at that pixel on each ligation. Every such number that occurred on at least 50 connected pixels in the image was determined to be a bead, represented by the centroid of the connected cluster.

SOLiD barcodes were then mapped to Illumina barcodes using a custom-built Matlab application that identified the pairwise distance between all members of the two sets of barcodes. Pairs of SOLiD barcodes and Illumina barcodes were saved for further analysis if the two barcodes were separated by at most two edits, and if the mapping between the barcodes was unique, i.e. if there were no other barcodes at equal or lower edit distance to either barcode.

### Cell Type Deconvolution:

A probability distribution across cell types was computed per bead using a custom method, implemented in Python, termed NMFreg (Non-Negative Matrix Factorization Regression). The method consisted of two main steps: first, single cell atlas data previously annotated with cell type identities was used to derive a basis in reduced gene space (via NMF), and second, non-negative least squares (NNLS) regression was used to compute the loadings for each bead in this basis. The details of the method are as follows.

As a preprocessing step, highly variable genes from single cell data were selected as in certain prior gene atlas studies. Only these genes were considered for future analysis. Beads were subsequently retained for analysis by NMFreg only if they had 5 transcripts in the set of variable genes. An interpretable low-dimensional basis for the space of highly variable genes was obtained as the set of K factors from performing NMF on the single cell atlas data. Each of the K factors/basis vector was mapped to a unique atlas cell type, yielding interpretability of the basis. The cell type identity of a factor was established as the most frequent cell type of atlas cells with highest loading in this factor.

With the aim of deriving a probability distribution over the atlas cell types for each Slide-seq bead, the beads loadings in the basis were first computed. This was achieved through NNLS regression of the Slide-seq bead by gene expression matrix onto the basis. The resulting bead by K matrix of loadings suffered from the well-known non-identifiability native to NMF, and a scaling of these loadings was customary before further utilizing them. Therefore, each of the K columns of the matrix of loadings was scaled to have L2 norm equal to 1. Afterwards, per bead, a cell type loading was computed as the L2 length of the loadings of all factors mapped to this atlas cell type. This yielded a bead by number of cell types matrix, in which each row was normalized to sum up to one. The result contained the desired probability distribution across cell types for each bead.

For certain computations, rather than requiring that beads had at least 5 transcripts of variable genes, instead beads were required to have at least 100 transcripts. This decreased the number of beads called by 72.6%+/−13.7% (mean+/−std over 7 cerebellar pucks). With this threshold, 56.3%+/−6.3% of beads passed the confidence threshold, a reduction compared to the number of beads that passed the confidence threshold without the 100 transcript filter (see below).

### Confidence Thresholding:

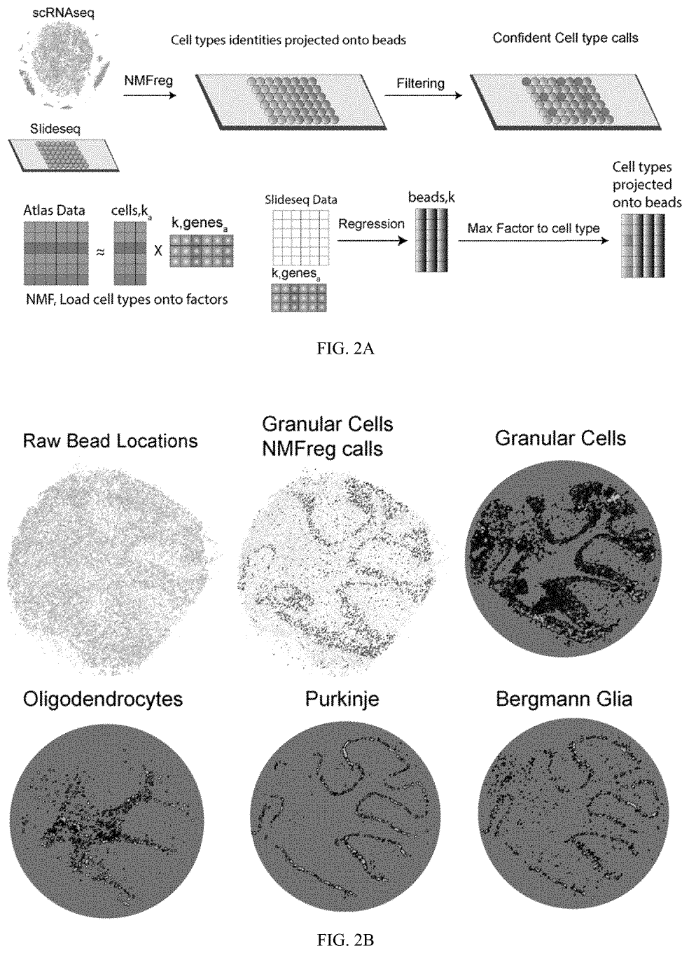

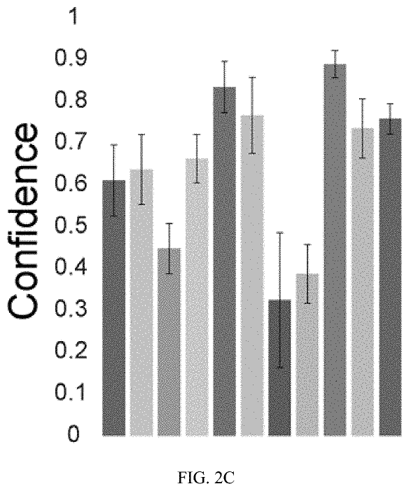

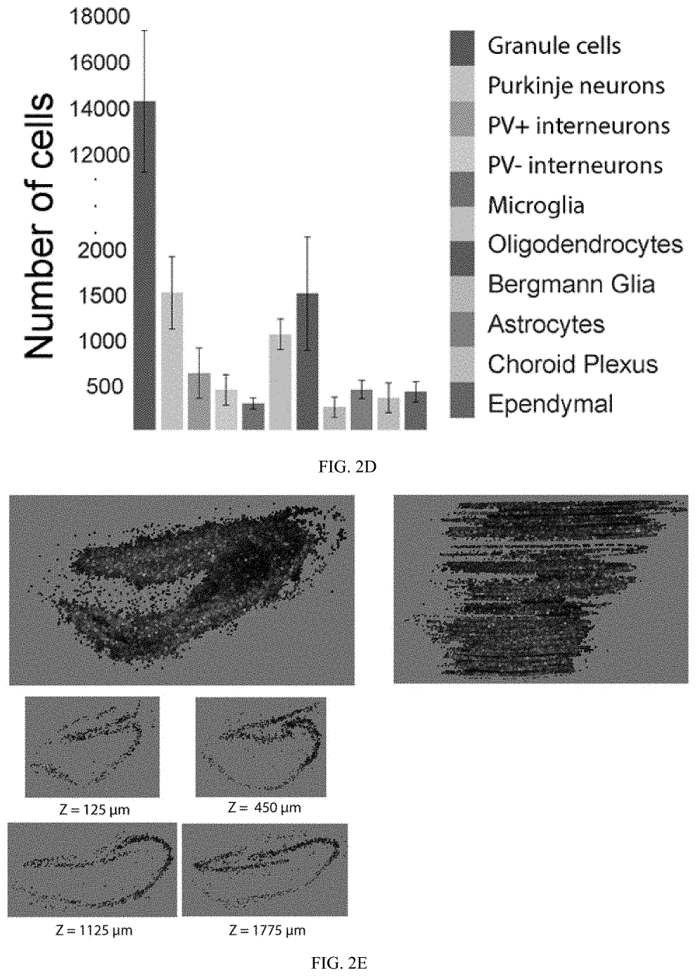

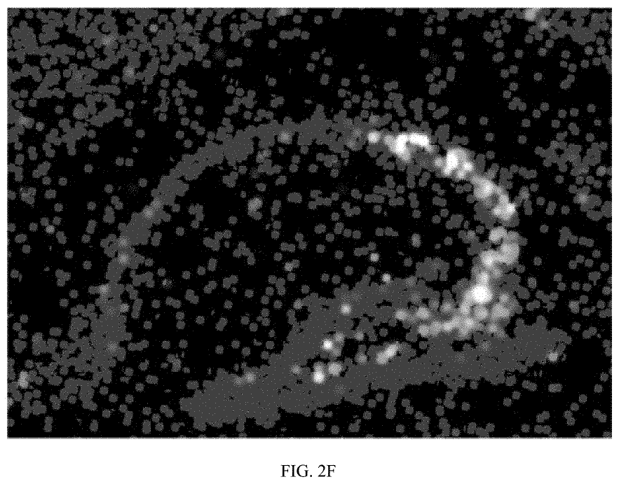

The bead factor loadings returned by NMFreg were in general less pure than the factor loadings obtained for single-cell sequencing data, likely reflecting both the sparsity of the Slide-Seq data and RNA contributions of other adjacent cell types. To determine whether a given bead could be confidently assigned to a single cell type, as in FIG. 2C, the L2 length of the vector of factor loadings was first calculated for factors representing the cell type to which the bead was assigned. For each cell-type, the minimum such L2 length appearing among Dropseq beads assigned to that cell type in the atlas data was also identified. The Slide-Seq bead was then said to be assigned confidently to the cell type if the L2 length of cell-type-specific factors for the Slide-Seq bead was at least as large as the smallest L2 length of cell-type-specific factors appearing among Dropseq beads assigned to the same cell type.

Interestingly, there was no relationship between the number of UMIs per bead and the confidence score of the bead, likely because beads with more UMIs were more likely to have multiple cells on them.

### Density Plots:

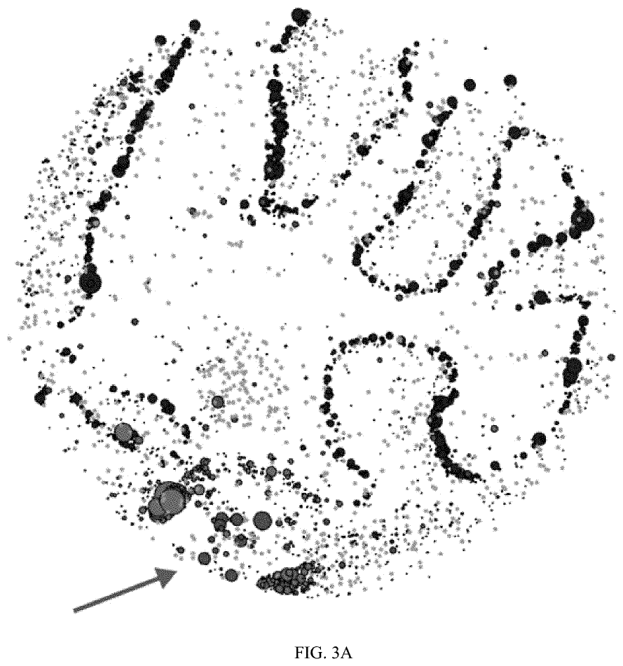

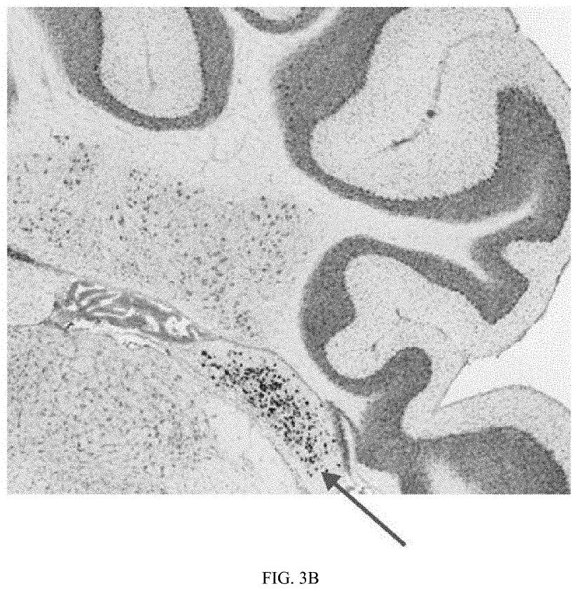

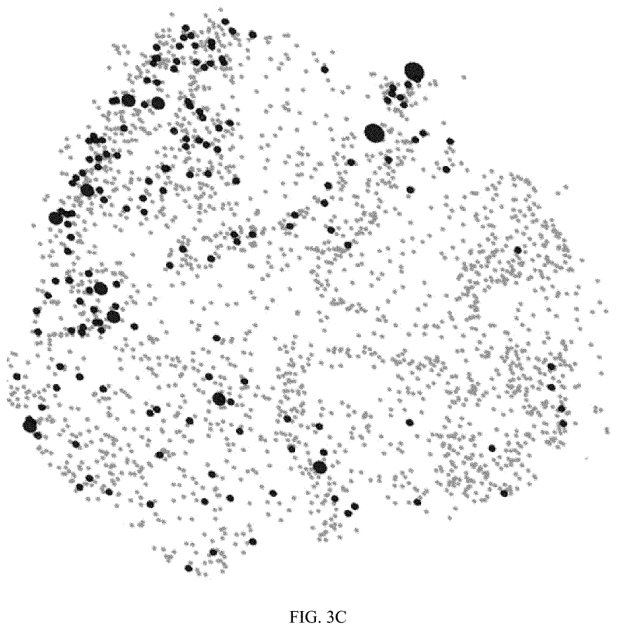

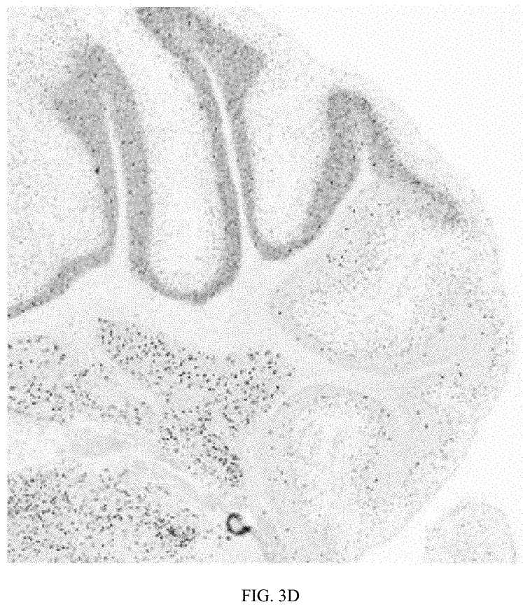

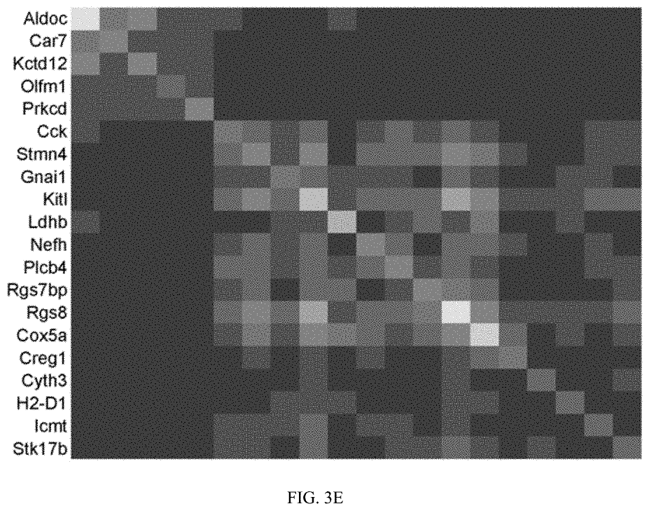

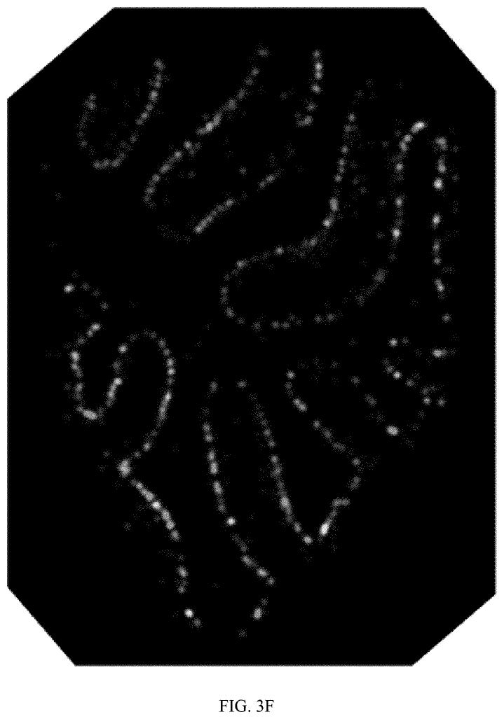

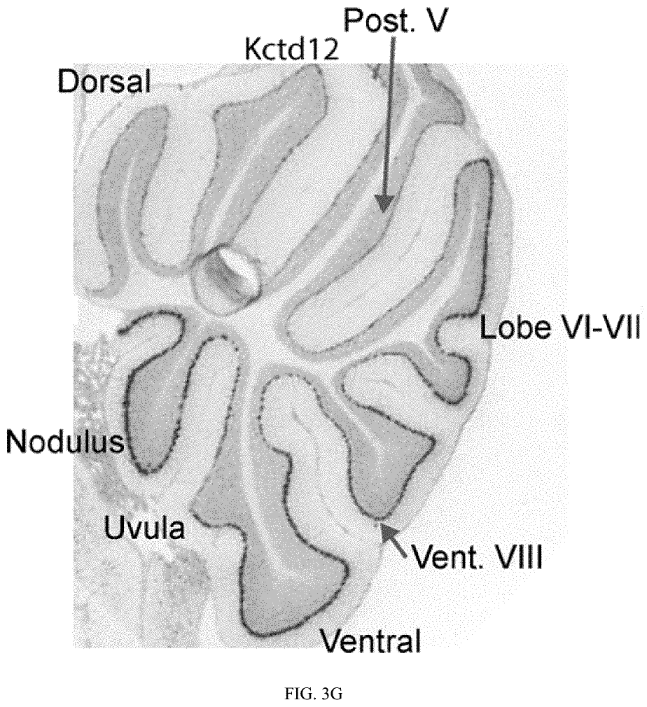

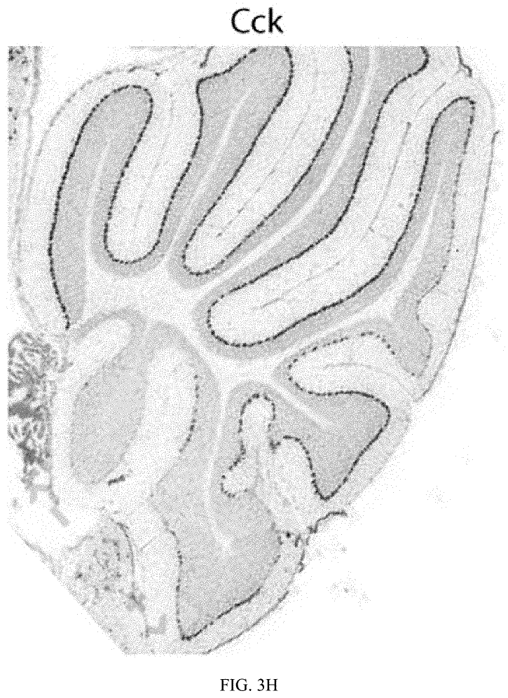

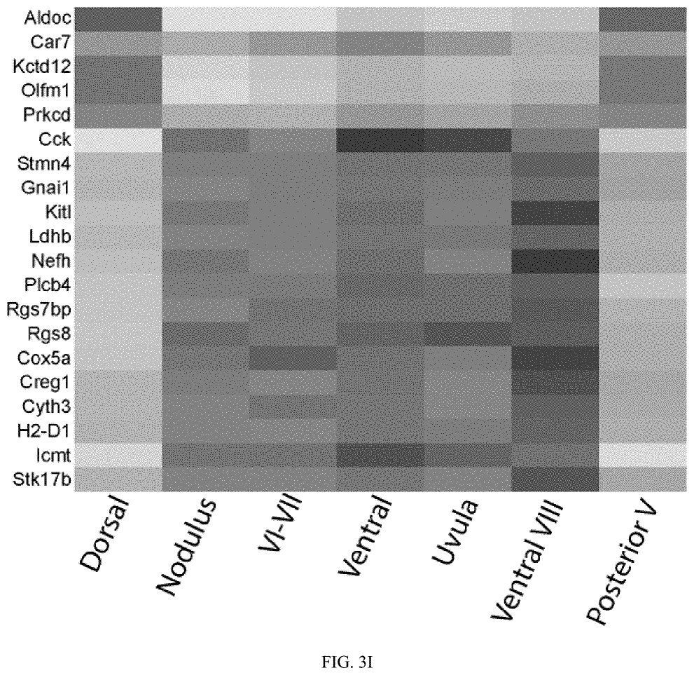

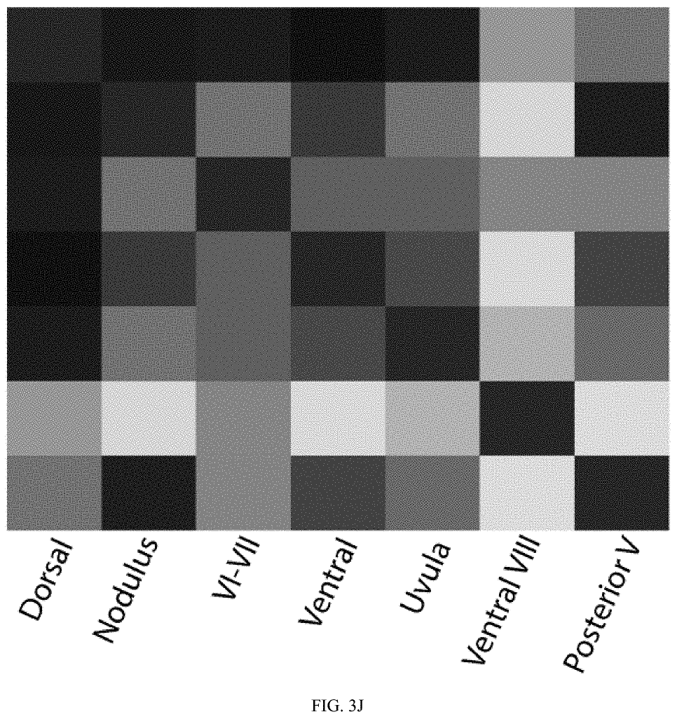

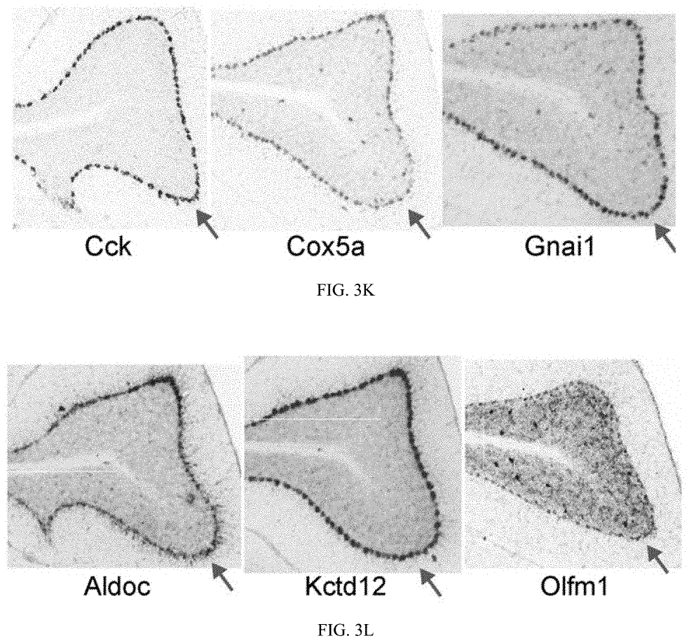

For the density plot images in FIGS. 2B (black backgrounds) and 3F, an image was as follows. Each point P in the 6030×6030 images was assigned an intensity equal to the sum of the intensities of all beads with centroids lying within 44-pixel square centered on P. For FIG. 2B (black backgrounds), each bead assigned to the indicated NMFreg cluster was assigned a unit intensity, while the intensity for each bead in FIG. 3F was taken as the total number of transcripts belonging to genes in the indicated metagene. Finally, the images were passed through Gaussian filters with a standard deviation of 12 pixels.

### Tissue Handling:

Fresh frozen tissue was warmed to −20° C. in a cryostat (Leica CM3050S) for 20 minutes prior to handling. Tissue was then mounted onto a cutting block with OCT and sliced at a 5 degree cutting angle at 10 μm thickness. Both OCT embedded and non-OCT embedded samples have been used for the instant procedure and equal yields have been observed in recovery of transcripts. Pucks were then placed on the cutting stage and tissue was maneuvered onto the pucks. The tissue was then melted onto the puck by moving the puck off the stage and placing a finger on the bottom side of the glass. The puck was then removed from the cryostat and placed into a 1.5 ml eppendorf tube. The sample library was then prepared as below. The remaining tissue was redeposited at −80° C. and stored for processing at a later date.

### Library Preparation:

Pucks in 1.5 mL tubes were immersed in 200 μL of hybridization buffer (6×SSC with 2 U/uL Lucigen NxGen RNAse inhibitor) for 15 minutes at room temperature to allow for binding of the RNA to the oligos on the beads. Subsequently, first strand synthesis was performed by incubating the pucks in RT solution for 1 hour at 42° C.

### RT Solution:

75 μl H2O

40 μl Maxima 5× RT Buffer (Thermofisher, EP0751)

40 μl 20% Ficoll PM-400 (Sigma, F4375-10G)

20 μl 10 mM dNTPs (NEB N0477L)

5 μl RNase Inhibitor (Lucigen 30281)

10 μl 50 μM Template Switch Oligo (Qiagen #339414YC00076714)

10 μl Maxima H-RTase (Thermofisher, EP0751)

200 μL of 2× tissue digestion buffer was then added directly to the RT solution and the mixture was incubated at 37 C for 40 minutes.

### 2× Tissue Digestion Buffer:

200 mM Tris-Cl pH 8

400 mM NaCl

4% SDS

10 mM EDTA

32 U/mL Proteinase K (NEB P8107S)

The solution was then pipetted up and down vigorously to remove beads from the surface, and the glass substrate was removed from the tube using forceps and discarded. 200 μl of Wash Buffer was then added to the 400 μl of tissue clearing and RT solution mix and the tube was then centrifuged for 3 minutes at 3000 RCF. The supernatant was then removed, the beads were resuspended in 200 μL of Wash Buffer, and were centrifuged again. After repeating this procedure an additional 2 times, the beads were moved into a 200 μL PCR strip tube, pelleted in a minifuge, and resuspended in 200 μL of water. The beads were then pelleted and resuspended in library PCR mix and PCRed.

### Wash Buffer:

10 mM Tris pH 8.0

1 mM EDTA

0.01% Tween-20

### Library PCR Mix:

23 μl H20

25 μl of 2× Kapa Hifi Hotstart ready mix (Kapa Biosystems KK2601)

1 μl of 100 μm Truseq PCR handle primer (IDT)

1 μl of 100 μm SMART PCR primer (IDT)

### PCR Program:

95 C 3 minutes

4 cycles of:
98 C 20 s
65 C 45 s
72 C 3 min

9 cycles of:
98 C 20 s
67 C 20 s
72 C 3 min

Then:

72 C 5 min

4 C forever

The PCR product was then purified by adding 30 μl of Ampure XP (Beckman Coulter A63880) beads to 50 μl of PCR product. The samples were cleaned according to manufacturer's instructions and resuspended into 10 ul of water. 1 μL of the resulting sample was run on an Agilent Bioanalyzer High sensitivity DNA chip (Agilent 5067-4626) for quantification of the library. Then, 600 pg of PCR product was taken from the PCR product and prepared into Illumina sequencing libraries through tagmentation with Nextera XT kit (Illumina FC-131-1096). Tagmentation was performed according to manufacturer's instructions and the library was amplified with primers Truseq5 and N700 series barcoded index primers. The PCR program was as follows:
72° C. for 3 minutes
95° C. for 30 seconds
12 cycles of:

95° C. for 10 seconds

55° C. for 30 seconds

72° C. for 30 seconds

72° C. for 5 minutes

### Hold at 10° C.

Samples were cleaned with AMPURE XP (Beckman Coulter A63880) beads in accordance with manufacturer's instructions at a 0.6× bead/sample ratio (30 μL of beads to 50 μL of sample) and resuspended in 10 μL of water. Library quantification was performed using the Bioanalyzer. Finally, the library concentration was normalized to 4 nM for sequencing. Samples were sequenced on the Illumina NovaSeq S2 flowcell with 12 samples per run (6 samples per lane) with the read structure 42 bases Read 1, 8 bases i7 index read, 50 bases Read 2. Each puck received approximately 200M-400M reads, corresponding to 3,000-5,000 reads per bead.

TABLE 1
Oligonucleotides used in this study.

| Name | Sequence |
|---|---|
| Truseq5 | AATGATACGGCGACCA |
| | CCGAGATCTACACTCT |
| | TTCCCTACACGACGC |
| | TCTTCCGATCT |
| | (SEQ ID NO: 3) |
| Smart PCR primer | AAGCAGTGGTATCAAC |
| | GCAGAGT |
| | (SEQ ID NO: 4) |
| Truseq PCR handle | CTACACGACGCTCTTC |
| | CGATCT |
| | (SEQ ID NO: 5) |
| Template Switch | AAGCTGGTATCAACGC |
| Oligo (TSO) | AGAGTGAATrG+GrG |
| | (SEQ ID NO: 6) |

Note: “r” prior to base indicates RNA.
“+” indicates LNA (locked nucleic acid)

### Example 2: Stable Association of Individually Barcode-Tagged Microbeads with a Glass Slide Provided a High-Resolution Array for Transcriptome Capture

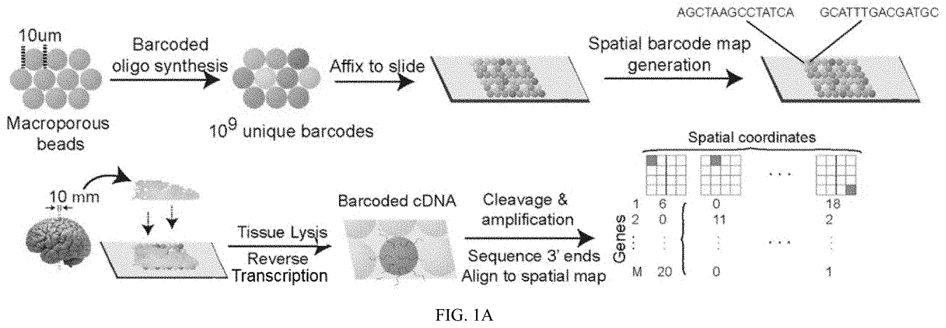

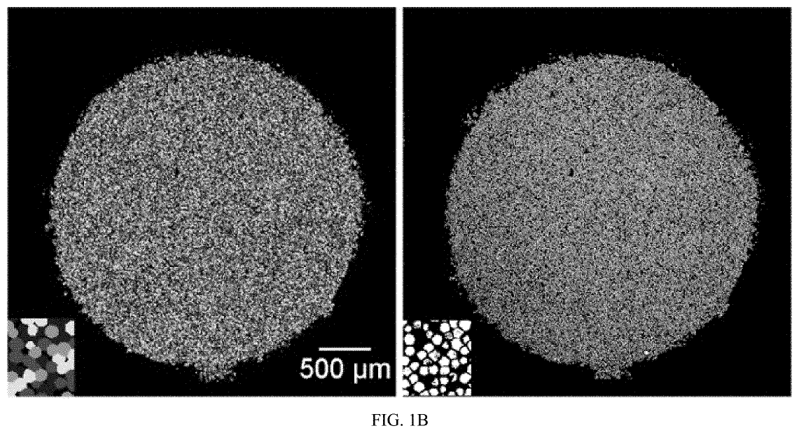

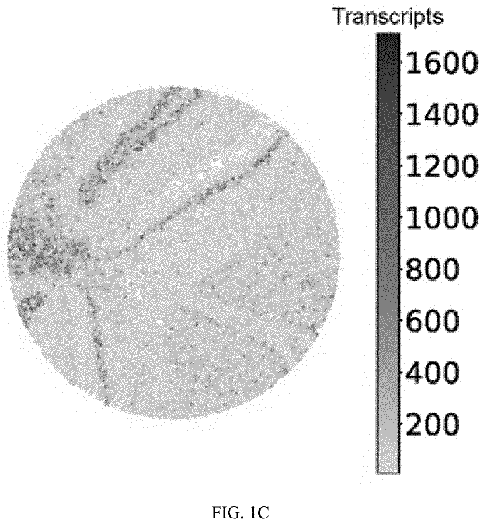

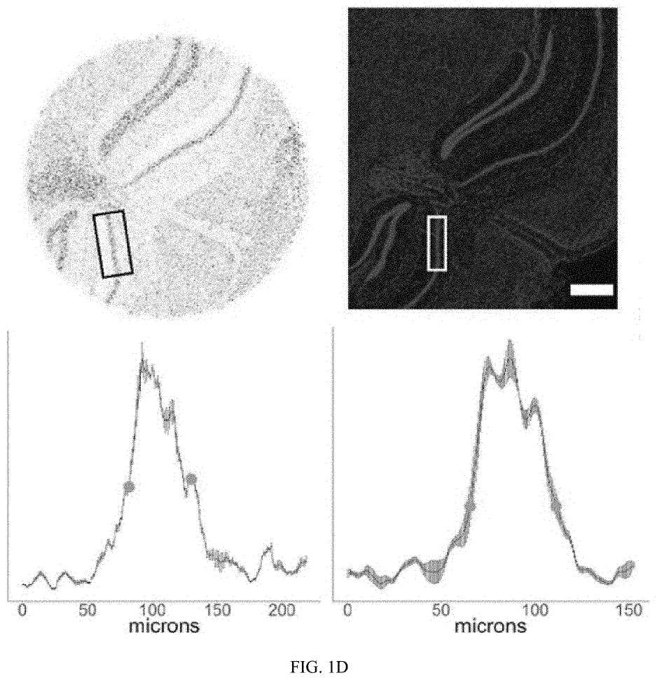

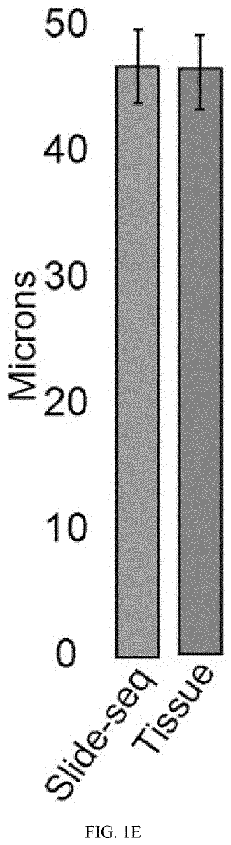

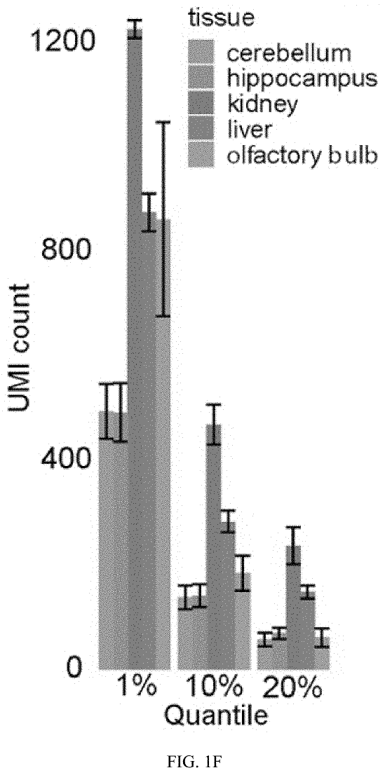

A large number of 10 μm beads that possessed unique nucleic acid barcodes were prepared via methods as described previously (e.g., as set forth in WO 2016/040476). Specifically, to generate a population of beads possessing individual barcodes that could be used for identification of an individual bead's position when arranged in a two-dimensional array as presently exemplified, polynucleotide synthesis was performed upon the surface of the beads in a pool-and-split fashion such that in each cycle of synthesis the beads were split into subsets that were subjected to different chemical reactions; and then this split-pool process was repeated in multiple cycles, to produce a combinatorially large number (approaching 4ⁿ) of distinct nucleic acid barcodes (FIG. 1A). Nucleotides were chemically built onto the bead material in a high-throughput manner, and the bead population that was used possessed approximately a billion (10⁹) unique bead-specific barcodes. After on-bead oligonucleotide synthesis, a glass slide was employed as a solid support for generation of an array of barcoded beads. To provide a capture material-coated surface for the bead array, the glass slide was initially coated with liquid electrical tape (applied as a liquid, the liquid tape dried to a vinyl polymer).

Barcoded beads as described above were applied to the capture material-coated slide, generating an array of beads in a dry condition (excess, non-captured beads were removed from the slide, thereby producing a single layer of captured beads). Because individually barcoded beads were deposited upon the capture material-coated surface in no pre-defined order, in situ sequencing of the bead array while captured upon the slide was performed, using the previously described SOLiD™ method (a sequencing-by-ligation technique that can be performed in situ upon a solid support-refer, e.g., to Voelkerding et al, Clinical Chem., 55-641-658, 2009; U.S. Pat. Nos. 5,912,148; 6,130,073, which are incorporated herein by reference in their entireties), thereby associating a bead's spatial barcode sequence with the two-dimensional location of that bead within the two-dimensional, slide-captured bead array (FIG. 1A).

The oligonucleotide-coated microbeads were thus attached to a glass slide surface as a two-dimensional solid support, and bead-attached oligonucleotide sequences were obtained within the spatial barcode sequence region for purpose of registering the respective locations of microbeads assorted throughout the array (in an exemplified bead-attached oligonucleotide sequence, each oligonucleotide respectively includes: a site of attachment (e.g., a cleavable site of bead attachment); a handle sequence (optionally, a universal handle sequence); a spatial barcode that is unique (or sufficiently unique) to each bead (as described above and as previously as noted); a unique molecular identifier (UMI); and 30 dT bases, which served as the capture region for the polyadenylated tails of mRNAs (referred to frequently in the literature as “oligo dr”)). This high-resolution bead array was then used for transcriptome capture from sample tissue, which was prepared as described in the below Example and elsewhere herein.

To develop Slide-seq, it was first examined whether barcodes could be arrayed randomly on a surface at high spatial resolution and their locations determined post-hoc. Split-pool synthesis barcoded oligonucleotide microparticles (‘beads’, 10 μm diameter), similar to those used by the Drop-seq approach to scRNA-seq (see, e.g., WO 2016/040476), were deposited onto a rubber-coated glass coverslip by evaporation, resulting in a packed bead surface which was termed a “puck” (88% packing). It was identified that the bead barcode sequences on the surface could be uniquely determined via in situ sequencing using the SOLiD sequencing-by-ligation chemistry (FIG. 1B).

### Example 3: A Glass Slide-Associated Barcode-Tagged Microbead Array Captured Transcriptomes with Robust Spatial Resolution

To determine if the surface could capture RNA with high resolution, a protocol was developed wherein frozen tissue sections (˜10 μm) were transferred onto the bead surface via cryosectioning (7). This process efficiently transferred RNA from the tissue to the surface, and subsequent processing of beads via standard single-cell library preparation pipelines generated 3′-end digital expression libraries. Performing this process on mouse hippocampal tissue slices, the distribution of transcripts across the puck was found to have recapitulated the distribution of cell bodies observed in the tissue (FIG. 1C). By comparing the width of CA1 observed in Slide-seq hippocampal data to that width observed in an adjacent, DAPI-stained tissue section (FIG. 1D), it was estimated that the length-scale of lateral diffusion of transcripts during hybridization was less than the width of an individual bead (FIG. 1E), which indicated that RNA was transferred from the tissue to the beads with high spatial resolution. Moreover, efficient capture was observed across a wide range of tissues, including brain, kidney, and liver (FIG. 1F).

To determine whether cell types from scRNA-seq could be faithfully mapped onto spatially localized Slide-seq data, a protocol termed NMF Regression (NMFReg) was developed, for projecting expression vectors from Slide-seq beads onto the linear subspace spanned by factors obtained from NMF of single-cell atlas data (FIG. 2A). Application of NMFreg to cerebellar Slide-seq data recapitulated the spatial distributions of classical cell-types, such as granule cells, Purkinje cells, and Oligodendrocytes (FIG. 2B). By comparing the loading on the maximum factor following projection to the distribution of factors in NMFReg, it was possible to identify beads that could be confidently assigned to a single cell-type. On average, 61.4%±5.1% of beads processed by NMFreg could be confidently assigned (mean±std, N=7 cerebellar pucks). This varied by cell type, with 88.8%±3.2% of beads called as choroid being called confidently (mean±std, N=7 pucks), while 32.4%±16.1% of beads called as Bergmann glia were called confidently (FIG. 2C). Moreover, the high spatial resolution of the method was found to be key for assigning beads to cell types with high confidence: upon artificially reducing the resolution of the method, the lower resolution images failed to confidently map cell types in regions that were heterogenous in cell types present, whereas homogenous regions such as the granular layer of the cerebellum maintained identifiability. Importantly, the representation of cell types in Slide-seq more accurately represented the natural distribution of cell types than single-cell sequencing. This was due to the sampling of tissue in native contexts allowing for better representation of rare cell types: whereas Purkinje neurons make up only 0.7% of cerebellar single-cell atlas data, they made up 7.8%±1.3% (mean±std, N=7 pucks) of a cerebellar puck, in line with expectation from histological studies (FIG. 2D).

The Slide-seq protocol was identified to be straightforward to execute, and pucks could be produced at high-throughput. To demonstrate the scalability of Slide-seq, it was applied to 70 tissue slices from a single dorsal mouse hippocampus, covering a volume of 39 cubic millimeters, with roughly 10 μm resolution in the dorsal-ventral and anterior-posterior axes, and ˜20 μm resolution in medial-lateral axis. This region contained an estimated ˜1 million beads that could be confidently assigned to single cell types. Pucks were computationally co-registered along the medial-lateral axis, allowing for visualization of gene expression in the hippocampus at high resolution in three dimensions (FIG. 2F). Metagenes comprised of markers for CA2 and for the hippocampal hilum were plotted on hippocampal pucks, and it was identified that they were highly expressed and specific for the expected regions (FIG. 2F), which confirmed the ability of Slide-seq to localize both common cell-types and more subtle cellular subtypes. The entire experimental processing for these 70 pucks (excluding the time and equipment required to make the pucks) required roughly 40 person-hours, and only standard experimental apparatus associated with cryosectioning and next generation sequencing. Thus, Slide-seq was readily scalable to the generation of three-dimensional atlases of spatial gene expression.

One key advantage that the Slide-seq approach has provided by allowing for spatial RNA sequencing with near-single-cell resolution is the ability to identify genes that are expressed in rare, spatially localized cell populations. The Slide-seq approach has therefore demonstrated particular power when it has been combined with a NMFReg algorithm, which has enabled the systematic identification of spatially localized cellular subpopulations, and spatial patterns of gene expression within known cell types. A nonparametric, kernel-free algorithm was previously developed to identify genes with spatially non-random distribution across the puck, where “random” was defined with reference to a null model in which transcripts were redistributed among beads while preserving the total number of transcripts per bead. A cluster of PV interneurons were identified in one corner of a coronal cerebellum puck that were marked by the little-studied gene Opioid Growth Factor Receptor Like 1 (Ogfrl1) (FIG. 3A), which was determined herein to be a highly specific marker for interneurons in the molecular and fusiform layers of the dorsal cochlear nucleus (FIG. 3B), also marked by Prkcd and Atp2b1. Without wishing to be bound by theory, this population was likely the cartwheel cells of the dorsal cochlear nucleus, which have been described previously as excited by the parallel fibers of the cochlear nucleus and have been believed to be involved in the generation of feedforward inhibition (8, 9). The existence of a specific genetic marker for this cell population is expected to enable controlling of the cell population genetically. The instant algorithm also identified Rasgrf1 as having significant nonrandom spatial distribution within the granule cell layer of the cerebellum (FIG. 3C), a pattern previously identified using ISH data (10) (FIG. 3D), thus validating the approach. Remarkably, however, a search for other genes with similar spatial distribution revealed no genes that were either correlated or uncorrelated with Rasgrf1, which indicated that if there were other genes with similar expression patterns to Rasgrf1, they were expressed at such low levels as to be undetectable by the Slide-seq process.

Whether the discovery of patterns of spatial gene expression in Slide-seq could be greatly assisted using patterns of correlation discovered in less sparse single-cell sequencing data was then examined. The cerebellum has been described as marked by parasagittal bands of gene expression in the Purkinje layer which are known to correlate both with the origins of afferents and targets of efferents (11). Several genes have been found to have similar or complementary parasagittal expression (12-15), but a systematic classification of banded gene patterns has been heretofore lacking. The significant gene calling algorithm of the instant disclosure was applied to the beads marked by NMFreg as Purkinje cells in the cerebellum, and this approach successfully identified Aldoc, a canonical marker for cerebellar banding, as well as Cck, Plcb4, Nefh, and several other genes. Applying a spatial correlation detection algorithm (7) to these genes led to the identification of a total of 31 genes, which were found to cluster into two sets, one marked by Aldoc and one marked by Cck (FIG. 3E). These sets included several genes that were previously known to be involved in cerebellar patterning, as well as many genes not previously associated with cerebellar banding patterns, including Olfm1 in the Aldoc cluster and Creg1, Cox5a, and Itgb1bp1 in the Cck cluster. Metagenes were formed for each of the 31 genes consisting of all genes with a correlation greater than 0.3 in single-cell Purkinje data. In the sections that were examined, the Aldoc and Cck metagenes thus plotted revealed a clear pattern, with the Aldoc metagene concentrated in the ventral cerebellum, including the nodulus (lobule X) and the region between lobules VI and VII, and the Cck metagene concentrated dorsally, and excluded from those regions (FIG. 3F), patterns that were recapitulated in ISH data of similar sections (FIGS. 3G and 3H).

To investigate whether the Aldoc and Cck patterns could describe all of the variation in gene expression that was observed across the cerebellum, a cerebellar puck was divided into seven morphologically-defined regions (shown in FIG. 3G) and the expression of all 31 of the spatially localized metagenes above was quantified in all 7 regions (FIG. 3I). The correlation between metagene expression was then calculated in different subregions. Although all the other regions that were examined correlated significantly with either the bulk dorsal or bulk ventral expression, surprisingly, gene expression in the ventral horn of lobule VIII did not correlate with expression in any other region at the p<0.001 level (corresponding to Bonferonni-corrected p<0.05) (FIG. 3J). Examination of genes in the Allen ISH database supported this hypothesis: Cck was strongly expressed in lobule VIII in similar sections, but Cox5a (in the Cck cluster) was apparently downregulated on the ventral side of lobule VIII, whereas Gnai1 (also in the Cck cluster) was apparently upregulated there (FIG. 3K). Likewise, Aldoc and Kctd12 were expressed strongly in lobule VIII in similar sections, but Olfm1, which is in the Aldoc cluster, was excluded (FIG. 3L). This likely points to a unique pattern of expression for lobule VIII, which would distinguish it from the predominant Aldoc/Cck banding pattern of the cerebellum. Thus, the Slide-seq approach of the instant disclosure enabled the discovery of regions of tissue with differential gene expression that did not otherwise emerge from anatomical or single-cell sequencing analysis.

Thus, the Slide-seq as disclosed in PCT/US19/has enabled the spatial analysis of gene expression in frozen tissue with high spatial resolution and easy scalability to large tissue volumes. Combined with single cell atlas data, Slide-seq has been able to identify the positions of cell types in tissue, and to identify novel patterns of gene expression and the responses to perturbations within those cell populations. Slide-seq was therefore identified as capable of facilitating the identification of rare cell types and novel, spatially restricted patterns of gene expression that are difficult to isolate in single-cell sequencing.

### Example 4: Development of RNase H-Dependent PCR-Enabled T Cell Receptor Sequencing (rhTCRseq) with Extended TCR-End Sequence Reads on a NGS Platform with the Slide-Seq Approach

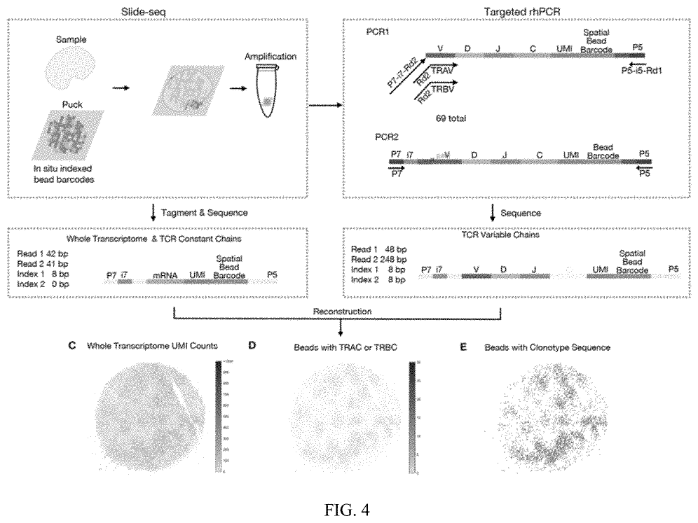

TCR transcript-targeted rhPCR was employed upon a Slide-seq cDNA sample (or a portion thereof) and extended read length sequences capable of resolving individual TCR variable regions were obtained (while also obtaining other identifying sequences within amplified cDNAs). Reconstructed images were obtained, which showed whole transcriptome UMI counts, beads with TRAC or TRBC, and beads with clonotype sequence (FIG. 4).

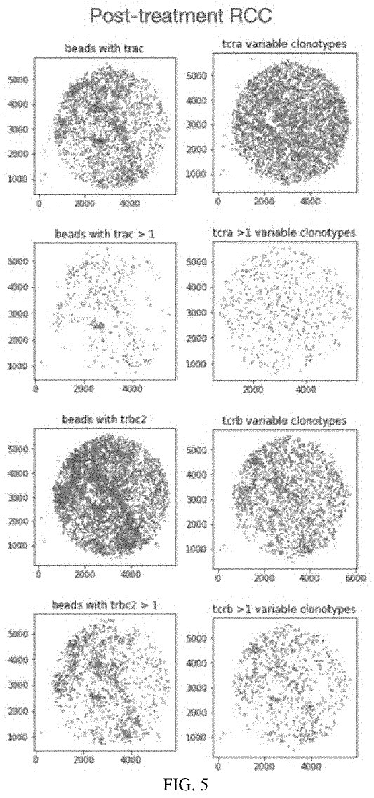

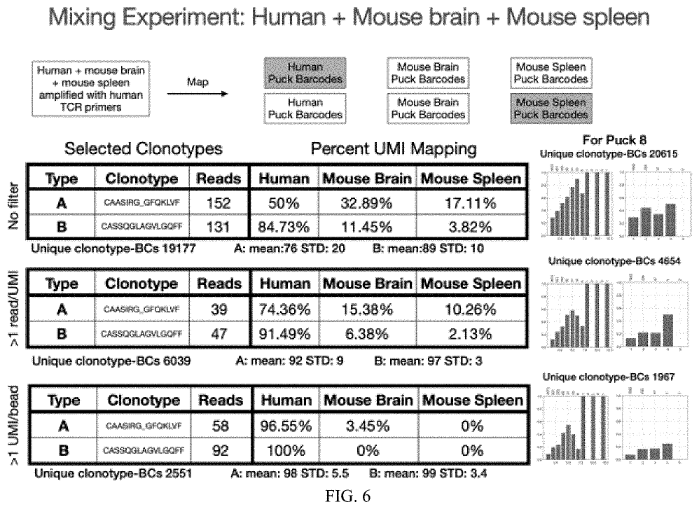

One unexpected issue confronted in attempting spatial resolution of TCR sequences was the prevalence of chimera formation between strands observed. Specifically, this issue was initially identified when the spatial locations of constant and variable regions did not match up in the human samples, which indicated that the variable spatial mapping was off (see FIG. 5). In an attempt to characterize this issue, experiments involving mixing of human RCC and mouse brain and mouse spleen puck libraries were performed, and rhTCR (rhPCR-mediated TCR enrichment) was performed upon the mixed sample (FIG. 6). The amount of barcode switching was quantified and was identified as very high, as clonotypes that should be human were often observed mapping to bead barcodes on the mouse pucks. These issues were ultimately overcome computationally by testing a few different computational filters, such as >1read/UMI and >1UMI/bead to reduce issues with random mixing. Capture was also improved by pulling the bead and UMI sequences from the constant region sequencing and automatically accepting single reads or UMIs if they matched those sequences. Emulsion PCR optimization has also been examined as a way to prevent mixing.

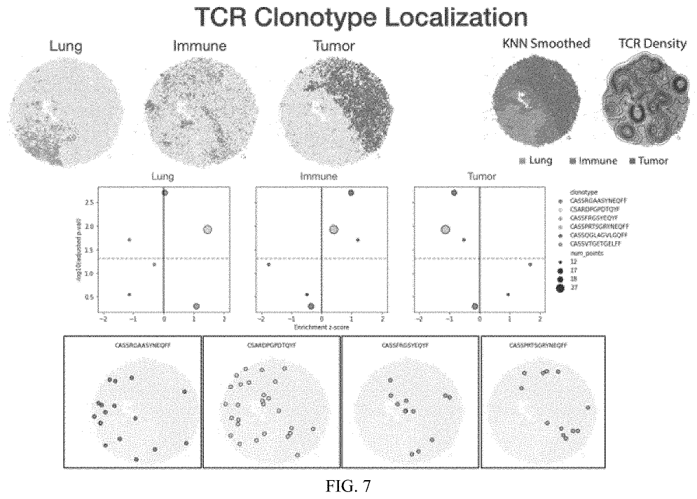

To analyze data obtained by such approaches, improved computational methods were developed. Specifically, unsupervised clustering was first performed, which identified a few regions of interest (lung, immune, tumor). Iterative k-Nearest Neighbors (KNN) clustering was performed to assign all remaining unassigned beads to one of those regions. p-values were then calculated for how spatially non-random the distribution of different T-cell clonotypes were in space, and it was discovered that several were spatially significant and had different enrichments in the different regions (FIG. 7). Spatially-resolved TCR clonotype information was thereby identified with levels of noise dramatically reduced (FIG. 7).

### Example 5: Combining RNase H-Dependent PCR-Enabled T Cell Receptor Sequencing (rhTCRseq) and Extended Read Sequencing on a NGS Platform with the Slide-Seq Approach Provide for Obtainment of Spatially-Resolvable Extended Length T-Cell Receptor Transcripts Together with Spatially-Resolvable Transcriptome Abundance Data

To demonstrate an improved approach for obtaining TCR sequence information using Slide-seq, a tissue section is prepared, while an array of immobilized beads attached to a solid surface is prepared as described above, with beads presenting oligonucleotides having spatial and other identifiers as described herein, as well as also including poly-dT tails of sufficient length to allow for capture of poly-A-tailed RNAs via hybridization from a sectioned sample. Bead identification sequences and associated two-dimensional positions on the solid support of individual beads attached to the solid support are obtained via a sequencing-by-ligation technique. Once such spatial information is obtained, a sectioned tissue sample is applied to the immobilized bead array and mRNA capture to the bead array occurs.

Bead-captured mRNAs of the tissue sample are reverse transcribed, thereby generating a population of cDNAs that carry spatial and molecular tag information also included within capture oligonucleotides. The cDNA population is PCR amplified in a manner that does not specifically enrich for TCR sequences. This amplified cDNA population is then split before being cleaved and tagged (“tagmented”) in preparation for sequencing.

To obtain spatially-resolvable T-cell receptor transcript sequences, a portion of the amplified cDNA population is contacted in solution with pairs of 3′-blocked oligonucleotides each containing a single ribonucleic acid base that are specific for relevant flanking sequences of T cell receptors, and the solution is subjected to RNase H-dependent PCR amplification, which thereby produces an amplified population of extended length T cell receptor sequences (including V, (D), J and C segments of each TCR transcript amplified) that also includes spatially-resolvable identifiers derived from capture bead oligonucleotides. rhPCR of the rhTCRseq process described in Li et al. (Nat. Protoc. 14: 2571-2594) is thereby performed, but in a spatially-resolvable manner. Optionally, cDNA amplification, rhPCR amplification, or both, can be performed as an emulsion PCR (ePCR) reaction, thereby limiting the extent of chimeric products formed during amplification (particularly relevant for resolution of individual, spatially resolvable TCR sequences).

The rhPCR-amplified TCR-selective spatially-resolvable DNA population and the PCR-amplified DNA population not specifically enriched for TCR sequences but including a spatially-resolvable representation of the transcriptome of the tissue are then prepared for sequencing, optionally after combining the populations at an appropriate mixed concentration to optimize concurrent identification of both spatially-resolvable TCR transcript sequences and spatially-resolvable transcriptome data during sequencing. In certain embodiments, solid phase reversible immobilisation (SPRI) paramagnetic beads can be employed in the presence of polyethylene glycol (PEG) to achieve an amplicon size-selection, which can be applied to rhPCR-amplified products, or to other PCR-amplified products, prior to preparing such nucleic acid populations for sequencing.

The amplified DNA populations (particularly those not specifically enriched for TCR sequences) are cleaved and tagged (tagmented) in preparation for sequencing, and sequence is obtained by a NGS method and associated instrumentation capable of obtaining extended read sequences, such as using the Illumina, Inc. (San Diego, Calif.) MiSeq® platform with sequencing parameters adjusted to obtain a much longer read on Read2, thereby allowing the MiSeq® platform to obtain individual TCR transcript sequence reads of sufficient length to span and resolve the TCR transcript variable regions in individual reads. Paired-end sequencing is also performed to identify bead identification sequences associated with all transcripts, including those bead identification sequences associated with individual TCR sequences.

Upon obtaining and processing sequence information at sufficient depth to identify not only spatially-resolvable extended length TCR transcript sequences but also spatially-resolvable transcriptome data, spatial resolution is performed upon both classes of data, and the data are then computationally assembled in two-dimensional space corresponding to tissue location. Representations of both TCR transcript sequences and transcriptome abundance in space (corresponding to near-single-cell resolution within the sectioned tissue) are then generated and evaluated. Such spatial data representations can also be overlaid for purpose of performing comparisons between identified TCR sequences and/or other transcripts.

Without limitation, it is expressly contemplated that the processes of the instant disclosure can be applied to tissues to study T-cell development as well as how T-cells with different T-cell receptor sequences respond differently to disease. Among other applications, the instant approaches can also be most directly commercially applied to develop and measure the success of immunotherapies.

### REFERENCES

1. A. Saunders et al., Molecular Diversity and Specializations among the Cells of the Adult Mouse Brain. Cell. 174, 1015-1030.e16 (2018).
2. S. Shah, E. Lubeck, W. Zhou, L. Cai, seqFISH Accurately Detects Transcripts in Single Cells and Reveals Robust Spatial Organization in the Hippocampus. Neuron. 94, 752-758.e1 (2017).
3. K. H. Chen, A. N. Boettiger, J. R. Moffitt, S. Wang, X. Zhuang, Spatially resolved, highly multiplexed RNA profiling in single cells. Science. 348 (2015).
4. E. Z. Macosko et al., Highly parallel genome-wide expression profiling of individual cells using nanoliter droplets. Cell. 161, 1202-1214 (2015).
5. A. M. Klein et al., Droplet barcoding for single-cell transcriptomics applied to embryonic stem cells. Cell. 161, 1187-1201 (2015).
6. P. L. Stahl et al., Visualization and analysis of gene expression in tissue sections by spatial transcriptomics. Science. 353, 78-82 (2016).
7. Materials and methods are available as supplementary materials online.
8. L. O. Trussell, D. Oertel, (Springer, Cham, 2018; http://link.springer.com/10.1007/978-3-319-71798-24), pp. 73-99.
9. M. T. Roberts, L. O. Trussell, Molecular Layer Inhibitory Interneurons Provide Feedforward and Lateral Inhibition in the Dorsal Cochlear Nucleus. J. Neurophysiol. 104, 2462-2473 (2010).
10. E. S. Lein et al., Genome-wide atlas of gene expression in the adult mouse brain. Nature. 445, 168-176 (2007).
11. C. Gravel, R. Hawkes, Parasagittal organization of the rat cerebellar cortex: Direct comparison of purkinje cell compartments and the organization of the spinocerebellar projection. J.

Comp. Neurol. 291, 79-102 (1990).
12. A. Demilly, S. L. Reeber, S. A. Gebre, R. V. Sillitoe, Neurofilament Heavy Chain Expression Reveals a Unique Parasagittal Stripe Topography in the Mouse Cerebellum. The Cerebellum. 10, 409-421 (2011).
13. N. H. Barmack, Z. Qian, J. Yoshimura, Regional and cellular distribution of protein kinase C in rat cerebellar Purkinje cells. J. Comp. Neurol. 427, 235-54 (2000).
14. G. Brochu, L. Maler, R. Hawkes, Zebrin II: A polypeptide antigen expressed selectively by purkinje cells reveals compartments in rat and fish cerebellum. J. Comp. Neurol. 291, 538-552 (1990).
15. J. R. Sarna, H. Marzban, M. Watanabe, R. Hawkes, Complementary stripes of phospholipase Cβ3 and Cβ4 expression by Purkinje cell subsets in the mouse cerebellum. J. Comp. Neurol. 496, 303-313 (2006).
16. P. D. Storer, K. J. Jones, Ribosomal RNA transcriptional activation and processing in hamster rubrospinal motoneurons: Effects of axotomy and testosterone treatment. J. Comp. Neurol. 458, 326-333 (2003).
17. K. L. Adams, V. Gallo, The diversity and disparity of the glial scar. Nat. Neurosci. (2017), doi:10.1038/s41593-017-0033-9.
18. A. M. Kenney, J. D. Kocsis, Peripheral axotomy induces long-term c-Jun amino-terminal kinase-1 activation and activator protein-1 binding activity by c-Jun and junD in adult rat dorsal root ganglia In vivo. J. Neurosci. 18, 1318-28 (1998).
19. G. A. Robinson, Immediate early gene expression in axotomized and regenerating retinal ganglion cells of the adult rat. Mol. Brain Res. 24, 43-54 (1994).
20. J. Honkaniemi, S. M. Sagar, I. Pyykonen, K. J. Hicks, F. R. Sharp, Focal brain injury induces multiple immediate early genes encoding zinc finger transcription factors. Mol. Brain Res. 28, 157-163 (1995).
21. Y. Lin et al., Activity-dependent regulation of inhibitory synapse development by Npas4.

Nature. 455, 1198-1204 (2008).
22. Q. Kong, M. P. Stockinger, Y. Chang, H. Tashiro, C. L. G. Lin, The presence of rRNA sequences in polyadenylated RNA and its potential functions. Biotechnol. J. 3, 1041-1046 (2008).

All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

One skilled in the art would readily appreciate that the present disclosure is well adapted to carry out the objects and obtain the ends and advantages mentioned, as well as those inherent therein. The methods and compositions described herein as presently representative of preferred embodiments are exemplary and are not intended as limitations on the scope of the disclosure. Changes therein and other uses will occur to those skilled in the art, which are encompassed within the spirit of the disclosure, are defined by the scope of the claims.

In addition, where features or aspects of the disclosure are described in terms of Markush groups or other grouping of alternatives, those skilled in the art will recognize that the disclosure is also thereby described in terms of any individual member or subgroup of members of the Markush group or other group.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Embodiments of this disclosure are described herein, including the best mode known to the inventors for carrying out the disclosed invention. Variations of those embodiments may become apparent to those of ordinary skill in the art upon reading the foregoing description.

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present disclosure provides preferred embodiments, optional features, modification and variation of the concepts herein disclosed may be resorted to by those skilled in the art, and that such modifications and variations are considered to be within the scope of this disclosure as defined by the description and the appended claims.

It will be readily apparent to one skilled in the art that varying substitutions and modifications can be made to the invention disclosed herein without departing from the scope and spirit of the invention. Thus, such additional embodiments are within the scope of the present disclosure and the following claims. The present disclosure teaches one skilled in the art to test various combinations and/or substitutions of chemical modifications described herein toward generating conjugates possessing improved contrast, diagnostic and/or imaging activity. Therefore, the specific embodiments described herein are not limiting and one skilled in the art can readily appreciate that specific combinations of the modifications described herein can be tested without undue experimentation toward identifying conjugates possessing improved contrast, diagnostic and/or imaging activity.

The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context. Those skilled in the art will recognize, or be able to ascertain using no more than routine experimentation, many equivalents to the specific embodiments of the disclosure described herein. Such equivalents are intended to be encompassed by the following claims.

We claim:

1. A method for obtaining from a tissue sample spatially-resolvable T cell receptor (TCR) sequence that spans TCR transcript variable regions, the method comprising:
(i) obtaining a tissue sample from a subject;
(ii) preparing a section of the tissue sample;
(iii) providing a solid support;
(iv) contacting the solid support with a capture material, thereby forming a capture material-coated solid support;
(v) contacting the capture material-coated solid support with a population of 1-100 μm diameter beads, wherein each bead has at least 1000 attached oligonucleotides and wherein at least one attached oligonucleotide of each bead each comprises: (a) a bead identification sequence that is common to all at least 1000 oligonucleotides on each bead and (b) a poly-dT tail of sufficient length to allow for capture of poly-A-tailed RNAs via hybridization, wherein the bead identification sequence that is common to all at least 1000 oligonucleotides on each bead is either a bead identification sequence that is unique to each bead within the population of 1-100 μm diameter beads or is a bead identification sequence that is a member of a population of bead identification sequences that is sufficiently degenerate to the population of 1-100 μm diameter beads that a majority of beads within the population of 1-100 μm diameter beads each possesses a unique bead identification sequence, thereby capturing a subpopulation of the population of 1-100 μm diameter beads upon the solid support;
(vi) identifying the bead identification sequence and associated two-dimensional position on the solid support of individual beads of the subpopulation of beads attached to the solid support;
(vii) contacting the subpopulation of 1-100 μm diameter beads captured upon the solid support with of the tissue sample;
(viii) performing a reverse transcription reaction upon poly-A-tailed RNAs captured by the bead subpopulation, thereby generating a cDNA population;
(ix) contacting a selection of or all of the cDNA population with (a) RNase H-dependent PCR primers designed for specific amplification of TCR-alpha and TCR-beta cDNAs and (b) RNase H, and performing PCR amplification upon the cDNA population, thereby generating a PCR-amplified nucleic acid population enriched for TCR-alpha and TCR-beta sequences; and
(x) obtaining sequence from the PCR-amplified nucleic acid population enriched for TCR-alpha and TCR-beta sequences using a sequencing process for TCR sequence-containing PCR-amplified nucleic acids having an average read length on at least one end in excess of 200 nucleotides, thereby obtaining TCR sequences that span TCR transcript variable regions for substantially all TCR sequences obtained and obtaining sequence from the PCR-amplified nucleic acid population enriched for TCR-alpha and TCR-beta sequences of bead identification sequences associated with TCR sequences,
thereby obtaining from the tissue sample spatially-resolvable T cell receptor (TCR) sequence that spans TCR transcript variable regions.

2. The method of claim 1, wherein each bead has at least 1000 attached oligonucleotides and wherein at least 100, optionally at least 1000, attached oligonucleotides of each bead each comprises: (a) a bead identification sequence that is common to all at least 1000 oligonucleotides on each bead and (b) a poly-dT tail of sufficient length to allow for capture of poly-A-tailed RNAs via hybridization.

3. The method of claim 2, wherein PCR amplification is performed upon the cDNA population of step (viii) in a manner that does not specifically enrich for TCR-alpha and TCR-beta sequences, thereby generating a PCR-amplified cDNA population that is not specifically enriched for TCR-alpha and TCR-beta sequences, wherein the PCR-amplified cDNA population that is not specifically enriched for TCR-alpha and TCR-beta sequences, or a subpopulation thereof, is the cDNA population contacted in step (ix) with (a) RNase H-dependent PCR primers designed for specific amplification of TCR-alpha and TCR-beta cDNAs and (b) RNase H, thereby generating a PCR-amplified nucleic acid population enriched for TCR-alpha and TCR-beta sequences.

4. The method of claim 2, wherein the cDNA population of step (viii) is partitioned into a first selection of the cDNA population that is contacted in step (ix) with RNase H-dependent PCR primers designed for specific amplification of TCR-alpha and TCR-beta cDNAs and RNase H, thereby generating a first PCR-amplified nucleic acid population that is enriched for TCR-alpha and TCR-beta sequences, and a second selection of the cDNA population, optionally wherein the second selection of the cDNA population is amplified with primers that are not selective for TCR sequence, thereby generating a second PCR-amplified nucleic acid population that is not enriched for TCR sequence relative to the cDNA population of step (viii).

5. The method of claim 3, wherein the PCR-amplified nucleic acid population enriched for TCR-alpha and TCR-beta sequences and the PCR-amplified nucleic acid population that is not specifically enriched for TCR-alpha and TCR-beta sequences are combined prior to obtaining sequence from the PCR-amplified nucleic acid population using a sequencing process having an average read length in excess of 200 nucleotides in step (x) for at least a TCR sequence-containing end of a TCR sequence-containing PCR-amplified nucleic acid, wherein sequences of non-TCR transcripts and associated bead identification sequences are thereby also obtained in step (x), wherein the method thereby obtains both spatially-resolvable T cell receptor (TCR) sequence that spans TCR transcript variable regions and spatially-resolvable transcript abundance information from the tissue sample.

6. The method of claim 1, wherein the PCR-amplified nucleic acid population enriched for TCR-alpha and TCR-beta sequences, and optionally the PCR-amplified nucleic acid population that is not specifically enriched for TCR-alpha and TCR-beta sequences, is cleaved and tagged prior to obtaining sequence from the PCR-amplified nucleic acid population in step (x).

7. The method of claim 1, wherein bead identification sequences associated with transcripts are obtained using paired-end sequencing, optionally wherein sequences of bead identification sequences associated with TCR sequences are obtained using paired-end sequencing.

8. The method of claim 1, wherein a subpopulation of the at least 1000 attached oligonucleotides of each bead comprises (a) a bead identification sequence that is common to all at least 1000 oligonucleotides on each bead and (b) a macromolecule-specific capture sequence that does not comprise a poly-dT tail.

9. The method of claim 8, wherein the macromolecule is selected from the group consisting of RNA, DNA and protein.

10. The method of claim 8, wherein the macromolecule-specific capture sequence comprises a gene-specific or transcript-specific sequence.

11. The method of claim 9, wherein the DNA is selected from the group consisting of a genomic DNA and a barcode DNA.

12. The method of claim 8, wherein the macromolecule-specific capture sequence is a component of a loaded transposase.

13. The method of claim 8, wherein a DNA barcode is used to capture an attached protein, optionally wherein the barcode-attached protein is an antibody, optionally wherein the antibody is specifically bound to a target protein, optionally wherein the antibody-bound target protein comprises a label.

14. The method of claim 8, further comprising PCR amplifying a nucleotide sequence of the captured macromolecule, thereby generating a PCR-amplified macromolecule nucleotide sequence population, and obtaining sequence from the PCR-amplified macromolecule nucleotide sequence population, thereby also obtaining spatially-resolvable macromolecule abundance data from the tissue sample.

15. The method of claim 1, wherein the PCR-amplified nucleic acid population comprising TCR-alpha and TCR-beta sequences is cleaved and tagged before obtaining sequence from the PCR-amplified nucleic acid population in step (x), optionally wherein a second PCR-amplified nucleic acid population is also cleaved and tagged before also obtaining sequence from the second PCR-amplified nucleic acid population.

16. The method of claim 1, wherein the obtaining sequence from the PCR-amplified nucleic acid population in step (x) is performed using a next-generation sequencing (NGS) method, optionally wherein the NGS sequencing method is selected from the group consisting of solid-phase, reversible dye-terminator sequencing; massively parallel signature sequencing; pyro-sequencing; sequencing-by-ligation; ion semiconductor sequencing; Nanopore sequencing and DNA nanoball sequencing, optionally wherein the next-generation sequencing approach is solid-phase, reversible dye-terminator sequencing.

17. The method of claim 1, wherein the obtaining sequence from the PCR-amplified nucleic acid population in step (x) is performed using a long read sequencing (LRS) method, optionally wherein the LRS method is selected from the group consisting of single molecule real time sequencing (SMRT) and nanopore sequencing.

18. The method of claim 1, wherein:
the average read length of the sequencing process exceeds about 850 nucleotides, optionally wherein the average read length of the sequencing process exceeds about 900 nucleotides, optionally wherein the average read length of the sequencing process exceeds about 950 nucleotides, optionally wherein the average read length of the sequencing process exceeds about 1000 nucleotides, optionally wherein the average read length of the sequencing process exceeds about 1050 nucleotides, optionally wherein the average read length of the sequencing process exceeds about 1100 nucleotides, optionally wherein the average read length of the sequencing process exceeds about 1150 nucleotides, optionally wherein the average read length of the sequencing process exceeds about 1200 nucleotides, optionally wherein the average read length of the sequencing process exceeds about 1250 nucleotides, optionally wherein the average read length of the sequencing process exceeds about 1300 nucleotides;
the tissue sample is obtained from a tissue selected from the group consisting of brain, lung, liver, kidney, pancreas, heart, spleen, lymph node, thymus and tumor;
the subject is a mammal, optionally a human;
the tissue sample is fixed, optionally wherein the tissue sample is fixed with a fixative selected from the group consisting of formalin, methanol, ethanol and acetone, optionally the tissue sample is a formalin-fixated and paraffin-embedded (FFPE) pathology specimen;
the solid support is a slide, optionally the solid support is a glass slide;
the capture material is applied as a liquid, optionally wherein the capture material is applied using a brush or aerosol spray, optionally wherein the capture material is a liquid electrical tape, optionally wherein the capture material dries to form a vinyl polymer, optionally wherein the vinyl polymer is polyvinyl hexane;
the 1-100 μm diameter beads comprise porous polystyrene, porous polymethacrylate and/or polyacrylamide;
the beads are 1-40 μm diameter beads, optionally wherein the beads are 10 μm beads;
the step of (vi) identifying the bead identification sequence and associated two-dimensional position on the solid support of individual beads of the subpopulation of beads attached to the solid support comprises performance of a sequencing-by-ligation technique;
the subpopulation of 1-100 μm diameter beads captured upon the solid support in step (vii) is maintained at a temperature between 4° C. and 30° C., optionally at about 25° C.;
step (vii) further comprises contacting the subpopulation of 1-100 μm diameter beads captured upon the solid support with a wash solution, optionally with a saline solution, optionally with a solution comprising between about 1M and about 3M NaCl, optionally with a saline-sodium citrate buffer comprising between about 1M and about 3M NaCl;
the bead identification sequence and associated two-dimensional position on the solid support of individual beads of the subpopulation of beads attached to the solid support is registered in a computer;
the method further comprises step (xi) generating an image of the tissue sample that depicts the location(s) and relative abundance of one or more captured TCRs or other captured macromolecules within the sample, optionally wherein the image is a two-dimensional image;
the hybridization is performed in 6×SSC buffer, optionally wherein the 6×SSC buffer is supplemented with detergent;
a selection of the beads possess primers against specific transcripts;
the barcoded array is reusable, optionally wherein cDNA is generated and then the second strand (carrying the barcode location) is synthesized, optionally wherein the second strand is capable of release from the array, optionally wherein the cDNA can be cleaved using a restriction enzyme to reveal a poly(A) tail on the array, thereby allowing for the array to be reused;
transcript-specific amplification of one or more transcripts other than TCR transcripts is also performed;
an array (puck) is physically transferred from one surface to another, optionally wherein a gel encasement is formed on top of the array (puck), thereby allowing beads to be picked up off the surface of the array (puck) without altering bead positions relative to each other;
the beads or array comprise or bind oligonucleotide-conjugated antibodies; and/or
the oligonucleotides having a poly-dT tail of sufficient length to allow for capture of poly-A-tailed RNAs via hybridization comprise unique molecular identifiers (UMIs), optionally wherein the UMIs of the hybridization probes are counted via sequencing to assess the levels of hybridization probe-bound macromolecules, optionally wherein the hybridization probe-bound macromolecules are selected from the group consisting of proteins, exons, transcripts, nucleic acid sequences comprising single nucleotide polymorphisms (SNPs) and/or genomic regions.

19. A method for obtaining from a tissue sample spatially-resolvable TCR sequence that spans TCR transcript variable regions and spatially-resolvable bulk poly-A-tailed RNA expression data, the method comprising:
(i) obtaining a tissue sample from a subject;
(ii) preparing a section of the tissue sample;
(iii) obtaining a solid support;
(iv) contacting the solid support with a capture material, thereby forming a capture material-coated solid support;
(v) contacting the capture material-coated solid support with a population of 1-100 μm diameter beads, wherein each bead has at least 1000 attached oligonucleotides and wherein at least 1000 attached oligonucleotides of each bead each comprises: (a) a bead identification sequence that is common to all at least 1000 oligonucleotides on each bead and (b) a poly-dT tail of sufficient length to allow for capture of poly-A-tailed RNAs via hybridization wherein the bead identification sequence that is common to all at least 1000 oligonucleotides on each bead is either a bead identification sequence that is unique to each bead within the population of 1-100 μm diameter beads or is a bead identification sequence that is a member of a population of bead identification sequences that is sufficiently degenerate to the population of 1-100 μm diameter beads that a majority of beads within the population of 1-100 μm diameter beads each possesses a unique bead identification sequence, thereby capturing a subpopulation of the population of 1-100 μm diameter beads upon the solid support;
(vi) identifying the bead identification sequence and associated two-dimensional position on the solid support of individual beads of the subpopulation of beads attached to the solid support;
(vii) contacting the subpopulation of 1-100 μm diameter beads captured upon the solid support with the section of the tissue sample;
(viii) performing a reverse transcription reaction upon poly-A-tailed RNAs captured by the bead subpopulation, thereby generating a cDNA population;
(ix) performing PCR amplification upon the cDNA subpopulation in a manner that does not specifically enrich for TCR-alpha and TCR-beta sequences, thereby generating a PCR-amplified nucleic acid population not specifically enriched for TCR-alpha and TCR-beta sequences;
(x) contacting the PCR-amplified nucleic acid population not specifically enriched for TCR-alpha and TCR-beta sequences, or a subpopulation thereof, with (a) RNase H-dependent PCR primers designed for specific amplification of TCR-alpha and TCR-beta cDNAs and (b) RNase H, and performing PCR amplification, thereby generating a PCR-amplified nucleic acid population enriched for TCR-alpha and TCR-beta sequences;
(xi) combining the PCR-amplified nucleic acid population enriched for TCR-alpha and TCR-beta sequences and the PCR-amplified nucleic acid population not specifically enriched for TCR-alpha and TCR-beta sequences into a single PCR-amplified nucleic acid population; and
(xii) obtaining sequence from the PCR-amplified nucleic acid population using a sequencing process for TCR sequence-containing PCR-amplified nucleic acids having an average read length on at least one end in excess of 200 nucleotides, thereby obtaining (a) TCR sequences that span TCR transcript variable regions for substantially all TCR sequences obtained; (b) sequences of bead identification sequences associated with TCR sequences; and (c) sequences of a population of poly-A-tailed RNAs bound to the bead oligonucleotides and associated bead identification sequences for sequenced poly-A-tailed RNAs,
thereby obtaining from the tissue sample spatially-resolvable T cell receptor (TCR) sequence that spans TCR transcript variable regions and spatially-resolvable bulk poly-A-tailed RNA expression data.

20. A method selected from the group consisting of:
A method for obtaining from a tissue sample spatially-resolvable TCR sequence that spans TCR transcript variable regions, the method comprising:
(i) generating a well array, wherein each well of the array can hold exactly one bead;
(ii) depositing beads into the wells of the well array, optionally by evaporation in a centrifuge;
(iii) brushing the well array to remove all of the beads not present in wells;
(iv) obtaining a tissue sample from a subject;
(v) preparing a section of the tissue sample;
(vi) depositing the section onto the well array and centrifuging, thereby forcing the section into the wells of the well array;
(vii) adding digestion buffer, thereby lysing the section and causing the RNA of cells of the section to transfer onto the beads in the wells;
(viii) performing a reverse transcription reaction upon the beads in the wells, thereby generating a cDNA population;
(ix) contacting a selection of or all of the cDNA population with (a) RNase H-dependent PCR primers designed for specific amplification of TCR-alpha and TCR-beta cDNAs and (b) RNase H, and performing PCR amplification upon the cDNA population, thereby generating a PCR-amplified nucleic acid population comprising TCR-alpha and TCR-beta sequences; and
(x) obtaining sequence from the PCR-amplified nucleic acid population using a sequencing process for TCR sequence-containing PCR-amplified nucleic acids having an average read length on at least one end in excess of 200 nucleotides, thereby obtaining TCR sequences that span TCR transcript variable regions for substantially all TCR sequences obtained and obtaining sequence from the PCR-amplified nucleic acid population of bead identification sequences associated with TCR sequences,
optionally further comprising removing beads from the wells by sonication or by photocleavage after step (vii), optionally before performing step (viii),
thereby obtaining from the tissue sample spatially-resolvable T cell receptor (TCR) sequence that spans TCR transcript variable regions;
A method for obtaining from a tissue sample spatially-resolvable TCR sequence that spans TCR transcript variable regions, the method comprising:
(i) obtaining a tissue sample from a subject;
(ii) preparing a section of the tissue sample;
(iii) obtaining a solid support;
(iv) adhering clusters of oligonucleotides in an array attached to the solid support, optionally wherein the array comprises barcoded clusters of oligonucleotides on a surface;
(v) identifying oligonucleotide cluster identification sequences and associated two-dimensional positions on the solid support of individual oligonucleotide clusters attached to the solid support, wherein the individual oligonucleotides are designed to capture RNA or DNA from the section of the tissue sample, optionally wherein at least one of the individual oligonucleotides of each cluster is designed for specific capture of TCR mRNA from the section of the tissue sample;
(vii) contacting the array with the section of the tissue sample;
(viii) performing RNase H-dependent PCR upon captured mRNAs of the section of the tissue sample, thereby generating a PCR-amplified DNA population comprising TCR-alpha and TCR-beta sequences; and
(ix) obtaining sequence from the PCR-amplified DNA population and an associated oligonucleotide cluster identification sequence for each DNA sequenced using a sequencing process for TCR sequence-containing PCR-amplified nucleic acids having an average read length on at least one end in excess of 200 nucleotides, thereby obtaining TCR sequences that span TCR transcript variable regions for substantially all TCR sequences obtained and obtaining sequence from the PCR-amplified DNA population of oligonucleotide cluster identification sequences associated with TCR sequences,
thereby obtaining from the tissue sample spatially-resolvable TCR sequence that spans TCR transcript variable regions;
A method for obtaining from a tissue sample spatially-resolvable TCR sequence that spans TCR transcript variable regions and macromolecule abundance data comprising:
(i) obtaining a tissue sample from a subject;
(ii) preparing a section of the tissue sample and adhering said section to a solid support;
(iii) forming an array of barcoded oligonucleotide clusters and/or an array of beads attached to barcoded oligonucleotides and contacting the section adhered to the solid support with the array;
(iv) identifying oligonucleotide cluster and/or bead array identification sequences and associated two-dimensional positions on the array of the barcoded oligonucleotide clusters and/or the array of beads attached to barcoded oligonucleotides; and
(v) obtaining the sequences of a population of macromolecules bound to the array(s) for each macromolecule sequenced, wherein the population of macromolecules comprises TCR RNA sequences, wherein TCR sequences are obtained by a process comprising RNase H-dependent PCR amplification of captured TCR RNA, thereby generating a PCR-amplified cDNA population comprising TCR-alpha and TCR-beta sequences, and obtaining sequence of the PCR-amplified cDNA population and an associated oligonucleotide cluster identification sequence for each cDNA sequenced using a sequencing process for TCR sequence-containing PCR-amplified nucleic acids having an average read length on at least one end in excess of 200 nucleotides, thereby obtaining TCR sequences that span TCR transcript variable regions for substantially all TCR sequences obtained and obtaining sequence from the PCR-amplified cDNA population of oligonucleotide cluster and/or bead array identification sequences associated with TCR sequences, thereby obtaining from the tissue sample spatially-resolvable TCR sequence that spans TCR transcript variable regions and macromolecule abundance data; and
A method for obtaining from a tissue sample spatially-resolvable T cell receptor (TCR) sequence that spans TCR transcript variable regions, the method comprising:
(i) obtaining a tissue sample from a subject;
(ii) preparing a section of the tissue sample;
(iii) providing a solid support;
(iv) contacting the solid support with a capture material, thereby forming a capture material-coated solid support;
(v) contacting the capture material-coated solid support with a population of 1-100 μm diameter beads, wherein each bead has at least 1000 attached oligonucleotides and wherein at least one attached oligonucleotide of each bead each comprises: (a) a bead identification sequence that is common to all at least 1000 oligonucleotides on each bead and (b) a poly-dT tail of sufficient length to allow for capture of poly-A-tailed RNAs via hybridization, wherein the bead identification sequence that is common to all at least 1000 oligonucleotides on each bead is either a bead identification sequence that is unique to each bead within the population of 1-100 μm diameter beads or is a bead identification sequence that is a member of a population of bead identification sequences that is sufficiently degenerate to the population of 1-100 μm diameter beads that a majority of beads within the population of 1-100 μm diameter beads each possesses a unique bead identification sequence, thereby capturing a subpopulation of the population of 1-100 μm diameter beads upon the solid support;
(vi) identifying the bead identification sequence and associated two-dimensional position on the solid support of individual beads of the subpopulation of beads attached to the solid support;
(vii) contacting the subpopulation of 1-100 μm diameter beads captured upon the solid support with of the tissue sample;
(viii) performing a reverse transcription reaction upon poly-A-tailed RNAs captured by the bead subpopulation, thereby generating a cDNA population;
(ix) contacting a selection of or all of the cDNA population with biotinylated probes capable of specifically annealing to TCR-alpha or TCR-beta sequences, and enriching for biotinylated probe-TCR complexes, thereby generating a nucleic acid population enriched for TCR-alpha and TCR-beta sequences; and
(x) obtaining sequence from the nucleic acid population enriched for TCR-alpha and TCR-beta sequences using a sequencing process for TCR sequence-containing nucleic acids having an average read length on at least one end in excess of 200 nucleotides, thereby obtaining TCR sequences that span TCR transcript variable regions for substantially all TCR sequences obtained and obtaining sequence from the nucleic acid population enriched for TCR-alpha and TCR-beta sequences of bead identification sequences associated with TCR sequences,
thereby obtaining from the tissue sample spatially-resolvable T cell receptor (TCR) sequence that spans TCR transcript variable regions.
